SEQUENCE LISTING (40 sequences)

SEQ ID NO: 1 - DNA, 286 bp, Artificial Sequence
  Synthetic
tacggtaaatggcccgcctggctgaccgcccaacgaccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgctt

SEQ ID NO: 2 - DNA, 265 bp, Artificial Sequence
  Synthetic
tctccccatctcccccccctccccacccccaattttgtatttatttattttttaattattttgtgcagcgatgggggcggggggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatc

SEQ ID NO: 3 - DNA, 247 bp, Artificial Sequence
  Synthetic
tgagctcacagaggggactttccgagagatctacagaggggactttccgagagcgagcttgggctgcaggtcgaccgtccatccattcacagcgcttctataaaggcgccagctgaggcgcctactactccaaccgcgactgcagcgagcaactgagaagactggatagagccggcggttccgcgaacgagcagtgaccgcgctcccacccagctctgctctgcagctccaccagtgtctctctaga

SEQ ID NO: 4 - DNA, 1030 bp, Artificial Sequence
  Synthetic
gaattgctcatcgcagcctgacggcgtaggttcatttcatttccacctagagcaagtgactgtaacgacacatttctttttgaatgttctcattcatgaaaggccttcactttaatattattacgaacatctaatttgtggacatcaagcgggctcctcacactctgaaaccagatacccccgagcccccgcaggtttttcttcctcattatcaggggtcccaggcatctggagcttaatgtgggatgtctccaacacaagcagggctagaacctcaaggcagaaggttgatgttgaagctatagaggggtgctacttactgacttgctcatcatggcattctcagcctgctgtcttatagaacccaagaccatggcccagggatggtagctcccacaatgggctaggctcttccccaccaaccacaagaaatgccctacaaccggatcttatggaggcattttctcaattgaggttttctccttccaagttgacataaaactaaccagacactcccccccaacacacacacacacccactggatgagcagagctgccctgaactgggcaaatgaggacattactgaacactccctccctggggtttggctcccgctctcaggggcagacactgaaaatcacaggctatgagagttggagcccagtttatcccaaaccgattttagatctctgtaccatgtctaggctatgcatagggttcttgtctctagggcgacccagtgctccagctgaggctgagagagagagagcacacataggagtggtggagcagcccctcagggaagaggtctggggccatgtcagagcctggcagtctccgattgtggcctctcgtccactcccagagccttgcccaggcagctgcaatcaccaaccacagcatcctttgggtttgacccactgagcacatgacccccaattagtcctggcagcatcccctgctcctcctgcttacatcagagagcacagagtagccgatataaatgctactggatgctggagggtgcag

SEQ ID NO: 5 - DNA, 1481 bp, Artificial Sequence
  Synthetic
gaaagggttttgtaacctgaaaattttcccactttactgttttaaaaataatatcattttaaatcaaagagttctactgtagtattttatttaacacaaaattgtattgcaatgcatgaaataaaatactgtttccaaaattgttttagagaaacacaaaggaagtcaataataaaagtggactaaagattcaacaaacagaaaaaatcagaaaggtaatagacttcaattcaccgtatgtgacgatcactaaatgtgcacgctccaataggaaggcaggaacagtctaaaatgtatgctgccactttaaacagtcttgcatgggaaagaatcagatatactacttacatagcattcataagaaaggtgacatggctacacataaatgtaaagtagaccagaatgtgaaattttatgttgatcaagaagggcatgttgtgatgataaaaggtcaatgtaccgagaagacataaatcgctatgtctcaagggagtttcaaggatgagaaaaactgacagaattcaaagaaataatagacaaatccagtcttccttgttagaaaactccatagtttctcagaattagacggaaaaaccattaacatggaggtcagaaagctggcagagacggttctggcactcaaggaattgaaaacactgtgtttctgttgactgggagacaaagctcttcctcttactgttactacacagaggcaaggctgtatgtaccattgtgtaaccaacactgacctgaaatttggaaaaagacaaacccagaagccagtaatgagaatcccttaactaatacgacaggatcgcttattcctgactttaaactagttctgtattagaagaggtaaggtaggtgacctagcggttcgggtgaagggcttcgttaagcatggtggcaagtttctacctacctggaaagcttgctatgagtgttacagagggggtgaagggcagagacaggtgcggtgactcggagaacagccaagaggaagcggttgttggaggcggccagaatgaggggaagtatagggctgggtaaaggaggtttgggggagccgtggacactacattgtgtagattctttagttacagttaaggaaaccccaacaggggtgcaccttgtgcaggcagggtccacagtggcgacaaggcagggtccactgaaggcgaaggacaaccttggcagggctatctagcgcctgcacgcaggtgctgcacccagagagctcagagcctggggacactcggacaggactccgcgcgggtctgaagcactcgtggaatgccaaaaccatctctgtcccgcgtcacggcagcccacgctcgggcaccccctggcggagctggtcggcggcgcgggggcaggtgccgggcggagccgggcgcacggggcgggacgaggcgggcggagggcggcggcgaccgccggtccacagaggccgcgcgcccagcccgcccgcaccgcgccat

SEQ ID NO: 6 - DNA, 786 bp, Artificial Sequence
  Synthetic
atggcgcccgtcgccgtctgggccgcgctggccgtcggactggagctctgggctgcggcgcacgccttgcccgcccaggtggcatttacaccctacgccccggagcccgggagcacatgccggctcagagaatactatgaccagacagctcagatgtgctgcagcaaatgctcgccgggccaacatgcaaaagtcttctgtaccaagacctcggacaccgtgtgtgactcctgtgaggacagcacatacacccagctctggaactgggttcccgagtgcttgagctgtggctcccgctgtagctctgaccaggtggaaactcaagcctgcactcgggaacagaaccgcatctgcacctgcaggcccggctggtactgcgcgctgagcaagcaggaggggtgccggctgtgcgcgccgctgcgcaagtgccgcccgggcttcggcgtggccagaccaggaactgaaacatcagacgtggtgtgcaagccctgtgccccggggacgttctccaacacgacttcatccacggatatttgcaggccccaccagatctgtaacgtggtggccatccctgggaatgcaagcatggatgcagtctgcacgtccacgtcccccacccggagtatggccccaggggcagtacacttaccccagccagtgtccacacgatcccaacacacgcagccaactccagaacccagcactgctccaagcacctccttcctgctcccaatgggccccagccccccagctgaagggagcactggcgacttcgctcttccagtt

SEQ ID NO: 7 - protein, 262 aa, Artificial Sequence
  Synthetic
MAPVAVWAALAVGLELWAAAHALPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDFALPV

SEQ ID NO: 8 - DNA, 279 bp, Artificial Sequence
  Synthetic
gatctggcagccttggagtgggttcctgccccctcgggcacacaaacagagctgaagaccaccctgggcacctccttggctggccgcatacctcctggcgggcagctgtgtacaaactacttgagagcaggtgttctggtggtacccacctgctctgtaatagtttgtacacagaggcctgcctggccctcgagagactgccctgactgaaggccctatcaggtgggggaggggatcctgatagagggcactgctgccactgttggggcccaagctgca

SEQ ID NO: 9 - DNA, 269 bp, Artificial Sequence
  Synthetic
ggcagccttggagtgggttcctgccccctcgggcacacaaacagagctgaagaccaccctgggcacctccttggctggccgcatacctcctggcgggcagctgtgtttcatcatagtacacacctctgttctggtggtacccagaggtgtgatccatgatgaaacacagaggcctgcctggccctcgagagactgccctgactgaaggccctatcaggtgggggaggggatcctgatagagggcactgctgccactgttggggcccaag

SEQ ID NO: 10 - DNA, 327 bp, Artificial Sequence
  Synthetic
tttgtcttttatttcaggtcccagatctagggctctgcgtttgctccaggtagtccgctgctcccttgggcctgggcccactgacagccctggtgcctctggccggctgcacacctcctggcgggcagctgtgtacaaactacttgagagcaggtgttctggcaatacctgcctgctctgtaatagtttgtacacggaggcctgccctgactgcccacggtgccgtggccaaagaggatctaagggcaccgctgagggcctacctaaccatcgtggggaataaggacagtgtcacccctgcaggggatccggtggtggtgcaaatca

SEQ ID NO: 11 - DNA, 325 bp, Artificial Sequence
  Synthetic
gtcttttatttcaggtcccagatcttagggctctgcgtttgctccaggtagtccgctgctcccttgggcctgggcccactgacagccctggtgcctctggccggctgcacacctcctggcgggcagctgtgtttccatggtaagttcaaggctgttctggcaatacctggccttgaagatgccatggaaacacggaggcctgccctgactgcccacggtgccgtggccaaagaggatctaagggcaccgctgagggcctacctaaccatcgtggggaataaggacagtgtcacccccctgcaggggatccggtggtggtgcaaat

SEQ ID NO: 12 - DNA, 269 bp, Artificial Sequence
  Synthetic
agggctctgcgtttgctccaggtagtccgctgctcccttgggcctgggcccactgacagccctggtgcctctggccggctgcacacctcctggcgggcagctgtgtaacgtcatgttagagatctttgttctggcaatacctgaagatctcatatatgacgttacacggaggcctgccctgactgcccacggtgccgtggccaaagaggatctaagggcaccgctgagggcctacctaaccatcgtggggaataaggacagtgtcaccc

SEQ ID NO: 13 - DNA, 269 bp, Artificial Sequence
  Synthetic
ggcagccttggagtgggttcctgccccctcgggcacacaaacagagctgaagaccaccctgggcacctccttggctggccgcatacctcctggcgggcagctgtgtaacgtcatgttagagatctttgttctggtggtacccaaagatctcatatatgacgttacacagaggcctgcctggccctcgagagactgccctgactgaaggccctatcaggtgggggaggggatcctgatagagggcactgctgccactgttggggcccaag

SEQ ID NO: 14 - DNA, 269 bp, Artificial Sequence
  Synthetic
agggctctgcgtttgctccaggtagtccgctgctcccttgggcctgggcccactgacagccctggtgcctctggccggctgcacacctcctggcgggcagctgtgacaggtaatagaagccatctttgttctggcaatacctgaagatggcaaccattacctgtcacggaggcctgccctgactgcccacggtgccgtggccaaagaggatctaagggcaccgctgagggcctacctaaccatcgtggggaataaggacagtgtcaccc

SEQ ID NO: 15 - DNA, 269 bp, Artificial Sequence
  Synthetic
ggcagccttggagtgggttcctgccccctcgggcacacaaacagagctgaagaccaccctgggcacctccttggctggccgcatacctcctggcgggcagctgtgacaggtaatagaagccatctttgttctggtggtacccaaagatggcaaccattacctgtcacagaggcctgcctggccctcgagagactgccctgactgaaggccctatcaggtgggggaggggatcctgatagagggcactgctgccactgttggggcccaag

SEQ ID NO: 16 - DNA, 269 bp, Artificial Sequence
  Synthetic
agggctctgcgtttgctccaggtagtccgctgctcccttgggcctgggcccactgacagccctggtgcctctggccggctgcacacctcctggcgggcagctgtgtttagtgacgtacaccattagtgttctggcaatacctgctaatggtcaatgtcactaaacacggaggcctgccctgactgcccacggtgccgtggccaaagaggatctaagggcaccgctgagggcctacctaaccatcgtggggaataaggacagtgtcaccc

SEQ ID NO: 17 - DNA, 269 bp, Artificial Sequence
  Synthetic
agggctctgcgtttgctccaggtagtccgctgctcccttgggcctgggcccactgacagccctggtgcctctggccggctgcacacctcctggcgggcagctgtgatccaccatcgctttctctgctgttctggcaatacctggcagagaatccaatggtggatcacggaggcctgccctgactgcccacggtgccgtggccaaagaggatctaagggcaccgctgagggcctacctaaccatcgtggggaataaggacagtgtcaccc

SEQ ID NO: 18 - DNA, 269 bp, Artificial Sequence
  Synthetic
ggcagccttggagtgggttcctgccccctcgggcacacaaacagagctgaagaccaccctgggcacctccttggctggccgcatacctcctggcgggcagctgtgtttagtgacgtacaccattagtgttctggtggtacccactaatggtcaatgtcactaaacacagaggcctgcctggccctcgagagactgccctgactgaaggccctatcaggtgggggaggggatcctgatagagggcactgctgccactgttggggcccaag

SEQ ID NO: 19 - DNA, 269 bp, Artificial Sequence
  Synthetic
ggcagccttggagtgggttcctgccccctcgggcacacaaacagagctgaagaccaccctgggcacctccttggctggccgcatacctcctggcgggcagctgtgatccaccatcgctttctctgctgttctggtggtacccagcagagaatccaatggtggatcacagaggcctgcctggccctcgagagactgccctgactgaaggccctatcaggtgggggaggggatcctgatagagggcactgctgccactgttggggcccaag

SEQ ID NO: 20 - DNA, 279 bp, Artificial Sequence
  Synthetic
catggggcagccttggagtgggttcctgccccctcgggcacacaaacagagctgaagaccaccctgggcacctccttggctggccgcatacctcctggcgggcagctgtgtttcatcatagtacacacctctgttctggtggtacccagaggtgtgatccatgatgaaacacagaggcctgcctggccctcgagagactgccctgactgaaggccctatcaggtgggggaggggatcctgatagagggcactgctgccactgttggggcccaagaagct

SEQ ID NO: 21 - DNA, 318 bp, Artificial Sequence
  Synthetic
gtcttttatttcaggtcccagatctggcagccttggagtgggttcctgccccctcgggcacacaaacagagctgaagaccaccctgggcacctccttggctggccgcatacctcctggcgggcagctgtgtttcatcatagtacacacctctgttctggtggtacccagaggtgtgatccatgatgaaacacagaggcctgcctggccctcgagagactgccctgactgaaggccctatcaggtgggggaggggatcctgatagagggcactgctgccactgttggggcccaagctgcaggggatccggtggtggtgc

SEQ ID NO: 22 - DNA, 325 bp, Artificial Sequence
  Synthetic
gtcttttatttcaggtcccagatcttagggctctgcgtttgctccaggtagtccgctgctcccttgggcctgggcccactgacagccctggtgcctctggccggctgcacacctcctggcgggcagctgtgattatcgctattgcagctttctgttctggcaatacctggaaagctggtacagcgataatcacggaggcctgccctgactgcccacggtgccgtggccaaagaggatctaagggcaccgctgagggcctacctaaccatcgtggggaataaggacagtgtcacccccctgcaggggatccggtggtggtgcaaat

SEQ ID NO: 23 - DNA, 325 bp, Artificial Sequence
  Synthetic
gtcttttatttcaggtcccagatcttagggctctgcgtttgctccaggtagtccgctgctcccttgggcctgggcccactgacagccctggtgcctctggccggctgcacacctcctggcgggcagctgtgtcagattatcgctattgcagctgttctggcaatacctggctgcaattccaataatctgacacggaggcctgccctgactgcccacggtgccgtggccaaagaggatctaagggcaccgctgagggcctacctaaccatcgtggggaataaggacagtgtcacccccctgcaggggatccggtggtggtgcaaat

SEQ ID NO: 24 - DNA, 269 bp, Artificial Sequence
  Synthetic
ggcagccttggagtgggttcctgccccctcgggcacacaaacagagctgaagaccaccctgggcacctccttggctggccgcatacctcctggcgggcagctgtgattatcgctattgcagctttctgttctggtggtacccagaaagctggtacagcgataatcacagaggcctgcctggccctcgagagactgccctgactgaaggccctatcaggtgggggaggggatcctgatagagggcactgctgccactgttggggcccaag

SEQ ID NO: 25 - DNA, 269 bp, Artificial Sequence
  Synthetic
ggcagccttggagtgggttcctgccccctcgggcacacaaacagagctgaagaccaccctgggcacctccttggctggccgcatacctcctggcgggcagctgtgtcagattatcgctattgcagctgttctggtggtacccagctgcaattccaataatctgacacagaggcctgcctggccctcgagagactgccctgactgaaggccctatcaggtgggggaggggatcctgatagagggcactgctgccactgttggggcccaag

SEQ ID NO: 26 - DNA, 279 bp, Artificial Sequence
  Synthetic
gatctagggctctgcgtttgctccaggtagtccgctgctcccttgggcctgggcccactgacagccctggtgcctctggccggctgcacacctcctggcgggcagctgtgtgacctctgtggcatcattcctgttctggcaatacctgggaatgatcgcgcagaggtcacacggaggcctgccctgactgcccacggtgccgtggccaaagaggatctaagggcaccgctgagggcctacctaaccatcgtggggaataaggacagtgtcacccctgca

SEQ ID NO: 27 - DNA, 269 bp, Artificial Sequence
  Synthetic
ggcagccttggagtgggttcctgccccctcgggcacacaaacagagctgaagaccaccctgggcacctccttggctggccgcatacctcctggcgggcagctgtgtgacctctgtggcatcattcctgttctggtggtacccaggaatgatcgcgcagaggtcacacagaggcctgcctggccctcgagagactgccctgactgaaggccctatcaggtgggggaggggatcctgatagagggcactgctgccactgttggggcccaag

SEQ ID NO: 28 - DNA, 325 bp, Artificial Sequence
  Synthetic
gtcttttatttcaggtcccagatcttagggctctgcgtttgctccaggtagtccgctgctcccttgggcctgggcccactgacagccctggtgcctctggccggctgcacacctcctggcgggcagctgtgatggtcttgttgttctccagctgttctggcaatacctggctggagatgagcaagaccatcacggaggcctgccctgactgcccacggtgccgtggccaaagaggatctaagggcaccgctgagggcctacctaaccatcgtggggaataaggacagtgtcacccccctgcaggggatccggtggtggtgcaaat

SEQ ID NO: 29 - DNA, 325 bp, Artificial Sequence
  Synthetic
gtcttttatttcaggtcccagatcttagggctctgcgtttgctccaggtagtccgctgctcccttgggcctgggcccactgacagccctggtgcctctggccggctgcacacctcctggcgggcagctgtgacgtctttggtctcaaaggggtgttctggcaatacctgcccctttgtcatcaaagacgtcacggaggcctgccctgactgcccacggtgccgtggccaaagaggatctaagggcaccgctgagggcctacctaaccatcgtggggaataaggacagtgtcacccccctgcaggggatccggtggtggtgcaaat

SEQ ID NO: 30 - DNA, 269 bp, Artificial Sequence
  Synthetic
ggcagccttggagtgggttcctgccccctcgggcacacaaacagagctgaagaccaccctgggcacctccttggctggccgcatacctcctggcgggcagctgtgatggtcttgttgttctccagctgttctggtggtacccagctggagatgagcaagaccatcacagaggcctgcctggccctcgagagactgccctgactgaaggccctatcaggtgggggaggggatcctgatagagggcactgctgccactgttggggcccaag

SEQ ID NO: 31 - DNA, 269 bp, Artificial Sequence
  Synthetic
ggcagccttggagtgggttcctgccccctcgggcacacaaacagagctgaagaccaccctgggcacctccttggctggccgcatacctcctggcgggcagctgtgacgtctttggtctcaaaggggtgttctggtggtacccacccctttgtcatcaaagacgtcacagaggcctgcctggccctcgagagactgccctgactgaaggccctatcaggtgggggaggggatcctgatagagggcactgctgccactgttggggcccaag

SEQ ID NO: 32 - DNA, 148 bp, Artificial Sequence
  Synthetic
atacatacttctttacattccaatacatacttctttacattccaatacatacttctttacattccaccatggactagtacaaacaccattgtcacactccaacaaacaccattgtcacactccaacaaacaccattgtcacactccaa

SEQ ID NO: 33 - DNA, 2347 bp, Artificial Sequence
  Synthetic
ctgcgcgctcgctcgctcactgaggccgcccgggcaaagcccgggcgtcgggcgacctttggtcgcccggcctcagtgagcgagcgagcgcgcagagagggagtgtagccatgctctaggaagatcaattcggtacaattcacgcgtcgacattgattattgactctggtcgttacataacttacggtaaatggcccgcctggctgaccgcccaacgaccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggccctagagtcgatcgaggaactgaaaaaccagaaagttaactggtaagtttagtctttttgtcttttatttcaggtcccagatctgtttaaacctgcaggggatccggtggtggtgcaaatcaaagaactgctcctcagtggatgttgcctttacttctaggcctgtacggaagtgttacttctgctctaaaagctgcggaattgtacccgcggccgatccaccggtgccaccatggcgcccgtcgccgtctgggccgcgctggccgtcggactggagctctgggctgcggcgcacgccttgcccgcccaggtggcatttacaccctacgccccggagcccgggagcacatgccggctcagagaatactatgaccagacagctcagatgtgctgcagcaaatgctcgccgggccaacatgcaaaagtcttctgtaccaagacctcggacaccgtgtgtgactcctgtgaggacagcacatacacccagctctggaactgggttcccgagtgcttgagctgtggctcccgctgtagctctgaccaggtggaaactcaagcctgcactcgggaacagaaccgcatctgcacctgcaggcccggctggtactgcgcgctgagcaagcaggaggggtgccggctgtgcgcgccgctgcgcaagtgccgcccgggcttcggcgtggccagaccaggaactgaaacatcagacgtggtgtgcaagccctgtgccccggggacgttctccaacacgacttcatccacggatatttgcaggccccaccagatctgtaacgtggtggccatccctgggaatgcaagcatggatgcagtctgcacgtccacgtcccccacccggagtatggccccaggggcagtacacttaccccagccagtgtccacacgatcccaacacacgcagccaactccagaacccagcactgctccaagcacctccttcctgctcccaatgggccccagccccccagctgaagggagcactggcgacttcgctcttccagtttgatgtacaagtaagcggccgcatacatacttctttacattccaatacatacttctttacattccaatacatacttctttacattccaccatggactagtacaaacaccattgtcacactccaacaaacaccattgtcacactccaacaaacaccattgtcacactccaaaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaattaggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 34 - DNA, 537 bp, Artificial Sequence
  Synthetic
atggaaatctgcagaggcctccgcagtcacctaatcactctcctcctcttcctgttccattcagagacgatctgccgaccctctgggagaaaatccagcaagatgcaagccttcagaatctgggatgttaaccagaagaccttctatctgaggaacaaccaactagttgctggatacttgcaaggaccaaatgtcaatttagaagaaaagatagatgtggtacccattgagcctcatgctctgttcttgggaatccatggagggaagatgtgcctgtcctgtgtcaagtctggtgatgagaccagactccagctggaggcagttaacatcactgacctgagcgagaacagaaagcaggacaagcgcttcgccttcatccgctcagacagtggccccaccaccagttttgagtctgccgcctgccccggttggttcctctgcacagcgatggaagctgaccagcccgtcagcctcaccaatatgcctgacgaaggcgtcatggtcaccaaattctacttccaggaggacgagtagtaa

SEQ ID NO: 35 - protein, 177 aa, Artificial Sequence
  Synthetic
MEICRGLRSHLITLLLFLFHSETICRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 36 - DNA, 2095 bp, Artificial Sequence
  Synthetic
ctgcgcgctcgctcgctcactgaggccgcccgggcaaagcccgggcgtcgggcgacctttggtcgcccggcctcagtgagcgagcgagcgcgcagagagggagtgtagccatgctctaggaagatcaattcggtacaattcacgcgtcgacattgattattgactctggtcgttacataacttacggtaaatggcccgcctggctgaccgcccaacgaccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggccctagagtcgatcgaggaactgaaaaaccagaaagttaactggtaagtttagtctttttgtcttttatttcaggtcccagatctgtttaaacctgcaggggatccggtggtggtgcaaatcaaagaactgctcctcagtggatgttgcctttacttctaggcctgtacggaagtgttacttctgctctaaaagctgcggaattgtacccgcggccgatccaccggtgccaccatggaaatctgcagaggcctccgcagtcacctaatcactctcctcctcttcctgttccattcagagacgatctgccgaccctctgggagaaaatccagcaagatgcaagccttcagaatctgggatgttaaccagaagaccttctatctgaggaacaaccaactagttgctggatacttgcaaggaccaaatgtcaatttagaagaaaagatagatgtggtacccattgagcctcatgctctgttcttgggaatccatggagggaagatgtgcctgtcctgtgtcaagtctggtgatgagaccagactccagctggaggcagttaacatcactgacctgagcgagaacagaaagcaggacaagcgcttcgccttcatccgctcagacagtggccccaccaccagttttgagtctgccgcctgccccggttggttcctctgcacagcgatggaagctgaccagcccgtcagcctcaccaatatgcctgacgaaggcgtcatggtcaccaaattctacttccaggaggacgagtagtaatgtacaagtaagcggccgcatacatacttctttacattccaatacatacttctttacattccaatacatacttctttacattccaccatggactagtacaaacaccattgtcacactccaacaaacaccattgtcacactccaacaaacaccattgtcacactccaaaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaattaggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 37 - DNA, 2383 bp, Artificial Sequence
  Synthetic
ctgcgcgctcgctcgctcactgaggccgcccgggcaaagcccgggcgtcgggcgacctttggtcgcccggcctcagtgagcgagcgagcgcgcagagagggagtgtagccatgctctaggaagatcaattcggtacaattcacgcgtcgacattgattattgactctggtcgttacataacttacggtaaatggcccgcctggctgaccgcccaacgaccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggccctagagtcgatcgaggaactgaaaaaccagaaagttaactggtaagtttagtctttttgtcttttatttcaggtcccagatctggcagccttggagtgggttcctgccccctcgggcacacaaacagagctgaagaccaccctgggcacctccttggctggccgcatacctcctggcgggcagctgtgtacaaactacttgagagcaggtgttctggtggtacccacctgctctgtaatagtttgtacacagaggcctgcctggccctcgagagactgccctgactgaaggccctatcaggtgggggaggggatcctgatagagggcactgctgccactgttggggcccaagctgcaggggatccggtggtggtgcaaatcaaagaactgctcctcagtggatgttgcctttacttctaggcctgtacggaagtgttacttctgctctaaaagctgcggaattgtacccgcggccgatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccatcaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaattaggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 38 - DNA, 1926 bp, Artificial Sequence
  Synthetic
ctgcgcgctcgctcgctcactgaggccgcccgggcaaagcccgggcgtcgggcgacctttggtcgcccggcctcagtgagcgagcgagcgcgcagagagggagtgtagccatgctctaggaagatcaattcggtacaattcacgcgtcgacattgattattgactctggtcgttacataacttacggtaaatggcccgcctggctgaccgcccaacgaccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggccctagagtcgatcgaggaactgaaaaaccagaaagttaactggtaagtttagtctttttgtcttttatttcaggtcccagatctagggctctgcgtttgctccaggtagtccgctgctcccttgggcctgggcccactgacagccctggtgcctctggccggctgcacacctcctggcgggcagctgtgtacaaactacttgagagcaggtgttctggcaatacctgcctgctctgtaatagtttgtacacggaggcctgccctgactgcccacggtgccgtggccaaagaggatctaagggcaccgctgagggcctacctaaccatcgtggggaataaggacagtgtcacccctgcaggggatccggtggtggtgcaaatcaaagaactgctcctcagtggatgttgcctttacttctaggcctgtacggaagtgttacttctgctctaaaagctgcggaattgtacccgcggccgatccaccggtcgccaccatggggcagccttggagtgggttcctgccccctcgggcacacaaacagagctgaagaccaccctgggcacctccttggctggccgcatacctcctggcgggcagctgtgtttcatcatagtacacacctctgttctggtggtacccagaggtgtgatccatgatgaaacacagaggcctgcctggccctcgagagactgccctgactgaaggccctatcaggtgggggaggggatcctgatagagggcactgctgccactgttggggcccaagaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaattaggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 39 - DNA, 1926 bp, Artificial Sequence
  Synthetic
ctgcgcgctcgctcgctcactgaggccgcccgggcaaagcccgggcgtcgggcgacctttggtcgcccggcctcagtgagcgagcgagcgcgcagagagggagtgtagccatgctctaggaagatcaattcggtacaattcacgcgtcgacattgattattgactctggtcgttacataacttacggtaaatggcccgcctggctgaccgcccaacgaccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggccctagagtcgatcgaggaactgaaaaaccagaaagttaactggtaagtttagtctttttgtcttttatttcaggtcccagatctagggctctgcgtttgctccaggtagtccgctgctcccttgggcctgggcccactgacagccctggtgcctctggccggctgcacacctcctggcgggcagctgtgtttcatcatagtacacacctctgttctggcaatacctggaggtgtgatccatgatgaaacacggaggcctgccctgactgcccacggtgccgtggccaaagaggatctaagggcaccgctgagggcctacctaaccatcgtggggaataaggacagtgtcacccctgcaggggatccggtggtggtgcaaatcaaagaactgctcctcagtggatgttgcctttacttctaggcctgtacggaagtgttacttctgctctaaaagctgcggaattgtacccgcggccgatccaccggtcgccaccatggggcagccttggagtgggttcctgccccctcgggcacacaaacagagctgaagaccaccctgggcacctccttggctggccgcatacctcctggcgggcagctgtgtacaaactacttgagagcaggtgttctggtggtacccacctgctctgtaatagtttgtacacagaggcctgcctggccctcgagagactgccctgactgaaggccctatcaggtgggggaggggatcctgatagagggcactgctgccactgttggggcccaagaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaattaggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 40 - DNA, 1926 bp, Artificial Sequence
  Synthetic
ctgcgcgctcgctcgctcactgaggccgcccgggcaaagcccgggcgtcgggcgacctttggtcgcccggcctcagtgagcgagcgagcgcgcagagagggagtgtagccatgctctaggaagatcaattcggtacaattcacgcgtcgacattgattattgactctggtcgttacataacttacggtaaatggcccgcctggctgaccgcccaacgaccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggccctagagtcgatcgaggaactgaaaaaccagaaagttaactggtaagtttagtctttttgtcttttatttcaggtcccagatctagggctctgcgtttgctccaggtagtccgctgctcccttgggcctgggcccactgacagccctggtgcctctggccggctgcacacctcctggcgggcagctgtgtgacctctgtggcatcattcctgttctggcaatacctgggaatgatcgcgcagaggtcacacggaggcctgccctgactgcccacggtgccgtggccaaagaggatctaagggcaccgctgagggcctacctaaccatcgtggggaataaggacagtgtcacccctgcaggggatccggtggtggtgcaaatcaaagaactgctcctcagtggatgttgcctttacttctaggcctgtacggaagtgttacttctgctctaaaagctgcggaattgtacccgcggccgatccaccggtcgccaccatggggcagccttggagtgggttcctgccccctcgggcacacaaacagagctgaagaccaccctgggcacctccttggctggccgcatacctcctggcgggcagctgtgtacaaactacttgagagcaggtgttctggtggtacccacctgctctgtaatagtttgtacacagaggcctgcctggccctcgagagactgccctgactgaaggccctatcaggtgggggaggggatcctgatagagggcactgctgccactgttggggcccaagaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaattaggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

(19) United States
(12) Patent Application Publication
(10) Pub. No.: US 2024/0002844 A1
(43) Pub. Date: Jan. 4, 2024

(54) DEVELOPMENT OF NOVEL GENE THERAPEUTICS FOR INFLAMMATION-INDUCED BONE LOSS

(71) Applicant: University of Massachusetts, Boston, MA (US)

(72) Inventors: Jae-Hyuck Shim, Worcester, MA (US); Guangping Gao, Worcester, MA (US); Jun Xie, Worcester, MA (US); Yeon-Suk Yang, Worcester, MA (US)

(73) Assignee: University of Massachusetts, Boston, MA (US)

(21) Appl. No.: 18/021,901

(22) PCT Filed: Aug. 18, 2021
(86) PCT No.: PCT/US2021/046447

§ 371 Date: Feb. 17, 2023

Related U.S. Application Data
(60) Provisional application No. 63/067,581, filed on Aug. 19, 2020.

(51) Int. Cl. C12N 15/113; C12N 15/86; A61P 29/00; A61P 19/02

(52) U.S. Cl. CPC C12N 15/113; C12N 15/86; C12N 2750/14143; A61P 19/02; C12N 2310/141; A61P 29/00

(57) ABSTRACT

Aspects of the disclosure relate to compositions and methods for reding inflammation and/or inhibiting bone loss (e.g., bone loss induced by inflammation). In some embodiments, the disclosure provides isolated nucleic acids and expression constructs (e.g., rAAVs, etc.) that encode one or more of the following transgenes: inhibitory nucleic acids targeting Schnurri 3 (SHN3), inhibitory nucleic acids targeting Cathepsin K (CTSK), inhibitory nucleic acids targeting sclerostin (SOST), inhibitory nucleic acids targeting receptor activator of NF-κβ (RANK), inhibitory nucleic acids targeting receptor activator of NF-κβ ligand (RANKL), soluble TNF-α Receptor 2 (sTNFR2), and soluble IL-1 Receptor Antagonist (sIL1Rα). In some embodiments, compositions described by the disclosure are useful for treating diseases associated with inflammation, for example rheumatoid arthritis (RA).

### RELATED APPLICATIONS

This application is a national stage filing under 35 U.S.C. § 371 of international PCT application PCT/US2021/046447, filed Aug. 18, 2021, which claims priority under 35 U.S.C. § 119(e) to U.S. provisional patent application, U.S. Ser. No. 63/067,581, filed Aug. 19, 2020, the entire contents of each of which are incorporated by reference herein.

Reference to a Sequence Listing Submitted as a Text File Via Efs-Web

The instant application contains a sequence listing which has been submitted in ASCII format via EFS-web and is hereby incorporated by reference in its entirety. Said ASCII copy, created on Dec. 6, 2022, is named U012070145US01-SEQ-KZM and is 41,482 bytes in size.

### BACKGROUND

In the setting of inflammatory arthritis such as rheumatoid arthritis (RA), inflammation activates osteoclasts (OCs) to resorb bone while simultaneously suppressing the ability of osteoblasts (OBs) to build bone. Patients with RA develop focal articular bone erosions and systemic bone loss resulting in osteopenia/osteoporosis. Most existing therapeutic agents that control bone loss act by inhibiting resorption of bone by osteoclasts (OCs), but these are accompanied by side effects, such as atypical fractures and osteonecrosis of the jaw.

### SUMMARY

Aspects of the disclosure relate to compositions and methods for reducing inflammation and/or inhibiting bone loss (e.g., bone loss induced by inflammation). The disclosure is based, in part, on isolated nucleic acids and expression constructs encoding one or more transgenes, such as inhibitory nucleic acids or proteins, that can simultaneously subdue inflammation and bone destruction, and promote healing of bone damage in the areas where inflammation is highly active in inflammatory arthritis, while limiting side effects in non-target tissues. In some embodiments, compositions described by the disclosure are useful for treating certain inflammatory diseases or conditions, for example rheumatoid arthritis (RA).

Accordingly, in some aspects, the disclosure provides an isolated nucleic acid comprising a transgene comprising an osteoclast (OC)-specific promoter or an osteoblast (OB)-specific promoter operably linked to a nucleic acid sequence encoding one or more inhibitory nucleic acids targeting sclerostin (SOST), schnurri-3 (SHN3), cathepsin K (CTSK), receptor activator of NF-κβ (RANK), and/or RANK ligand (RANKL).

In some embodiments, a transgene further comprises a nucleic acid encoding a protein. In some embodiments, a protein is a therapeutic protein. In some embodiments, a protein is a marker protein, for example green fluorescent protein (GFP). In some embodiments, a protein comprises soluble human tumor necrosis factor alpha receptor 2 (sTNRF2), a soluble IL-1 Receptor Alpha (sIL1Rα), or sTNRF2 and sIL1Rα.

In some embodiments, an OC-specific promoter comprises a NF-κβ promoter. In some embodiments, an OC-specific promoter comprises a RANK promoter. In some embodiments, a NF-κβ promoter is induced by inflammation. In some embodiments, a NF-κβ promoter is a PB2 promoter (SEQ ID NO: 3). In some embodiments, an OB-specific promoter comprises an osteocalcin (OCN) promoter (e.g. as set forth in SEQ ID NO: 4).

In some embodiments, at least one of the one or more inhibitory nucleic acids is a shRNA, miRNA, or artificial miRNA (ami-RNA). In some embodiments, an ami-RNA comprises a mouse miRNA backbone. In some embodiments, a miRNA backbone is a human miR-33 backbone.

In some embodiments, at least one inhibitory nucleic acids target SHN3. In some embodiments, the inhibitory nucleic acid is encoded by a nucleic acid comprising the sequence set forth in any one of SEQ ID NOs: 8-11.

In some embodiments, at least one inhibitory nucleic acid targets CTSK. In some embodiments, the inhibitory nucleic acid is encoded by a nucleic acid comprising the sequence set forth in any one of SEQ ID NOs: 20-25.

In some embodiments, at least one inhibitory nucleic acid targets SOST. In some embodiments, the inhibitory nucleic acid is encoded by a nucleic acid comprising the sequence set forth in any one of SEQ ID NOs: 26-31.

In some embodiments, at least one inhibitory nucleic acid targets RANK.

In some embodiments, at least one inhibitory nucleic acid targets RANKL. In some embodiments, the inhibitory nucleic acid is encoded by a nucleic acid comprising the sequence set forth in any one of SEQ ID NOs: 12-19.

In some aspects, the disclosure provides an isolated nucleic acid encoding a transgene which encodes a first inhibitory nucleic acid targeting a gene selected from SHN3, CTSK, SOST, RANK, and RANKL; and a second inhibitory nucleic acid targeting a gene selected from SHN3, CTSK, SOST, RANK, and RANKL.

In some embodiments, a transgene further comprises a promoter operably linked to the first inhibitory nucleic acid or the second inhibitory nucleic acid. In some embodiments, a promoter comprises a chicken beta-actin (CB) promoter. In some embodiments, a transgene further comprises a CMV enhancer sequence. In some embodiments, a promoter is an inducible promoter. In some embodiments, the inducible promoter is induced by inflammation in a subject (e.g., the expression or release of inflammatory cytokines in the subject). In some embodiments, an inducible promoter comprises a PB2 promoter.

In some embodiments, a transgene further encodes a protein. In some embodiments, a protein is a therapeutic protein. In some embodiments, a therapeutic protein comprises soluble human tumor necrosis factor alpha receptor 2 (sTNRF2), a soluble IL-1 Receptor Antagonist (sIL1Rα), or sTNRF2 and sIL1Rα.

In some embodiments, a transgene further comprises one or more miRNA binding sites. In some embodiments, least one of the miRNA binding sites is a miR-1 binding site or a miR-122 binding site.

In some embodiments, a transgene is flanked by adeno-associated virus (AAV) inverted terminal repeats (ITRs). In some embodiments, AAV ITRs are AAV2 ITRs.

In some aspects, the disclosure provides an isolated nucleic acid comprising or encoding a sequence set forth in any one of SEQ ID NOs: 1-40.

In some aspects, the disclosure provides a vector comprising an isolated nucleic acid as described herein. In some embodiments, a vector is a plasmid, bacmid, cosmid, viral, closed-ended linear DNA (ceDNA), or Baculovirus vector. In some embodiments, a vector is a recombinant adeno-associated virus (rAAV) vector, retroviral vector, or adenoviral vector.

In some aspects, the disclosure provides a recombinant adeno-associated virus (rAAV) comprising an isolated nucleic acid as described herein; and at least one AAV capsid protein.

In some embodiments, an AAV capsid protein is of a serotype selected from AAV1, AAV2, AAV2-TM, AAV3, AAV4, AAV5, AAV6, AAV6-TM, AAV6.2, AAV7, AAV8, AAV9, AAV.rh8, AAV.rh10, AAV.rh39, AAV. 43, AAV2/2-66, AAV2/2-84, and AAV2/2-125, or a variant of any of the foregoing.

In some embodiments, an AAV capsid protein transduces osteoblast cells (OB s), optionally wherein the capsid protein is of a serotype selected from AAV1, AAV4, AAV5, AAV6, AAV7, AAV9, AAVrh10, AAVrh39, or a variant of any of the foregoing.

In some embodiments, an AAV capsid protein transduces osteoclast cells (OCs), optionally wherein the capsid protein is of a serotype selected from AAV1, AAV5, AAV6, AAV6.2, AAV7, AAV8, AAV9, AAV10, AAV.rh39, and AAV.rh43, or a variant of any of the foregoing.

In some embodiments, an AAV capsid protein is a DSS.AAV9 (SEQ ID NO: 41) capsid protein.

In some aspects, the disclosure provides a composition comprising an rAAV as described herein, and a pharmaceutically acceptable excipient.

In some aspects, the disclosure provides a method for inhibiting bone loss in a subject, the method comprising administering to the subject an isolated nucleic acid, vector, or rAAV as described herein.

In some aspects, the disclosure provides a method for inhibiting inflammation in a joint of a subject, the method comprising administering to the subject an isolated nucleic acid, vector, or rAAV as described herein.

In some aspects, the disclosure provides a method for treating rheumatoid arthritis in a subject, the method comprising administering to the subject an isolated nucleic acid, vector, or rAAV as described herein.

In some embodiments, a subject is a mammal. In some embodiments, a subject is a human. In some embodiments, a subject has or is suspected of having an inflammatory condition. In some embodiments an inflammatory condition is rheumatoid arthritis (RA).

In some embodiments, administration to a subject occurs by injection. In some embodiments, injection is systemic injection (e.g., intravenous injection, etc.) or local injection (e.g. intramuscular (IM) injection, knee injection, and femoral intramedullary injection, etc.). In some embodiments, systemic injection comprises intravenous injection. In some embodiments, local injection comprises intramuscular (IM) injection. In some embodiments, local injection comprises knee injection. In some embodiments, local injection comprises femoral intramedullary injection.

In some embodiments, administration to a subject occurs by implantation of a tissue or graft comprising an rAAV as described herein into the subject.

In some embodiments, administration results in reduction in inflammatory cytokines and/or reduction of bone loss in the subject.

In some embodiments, administration results in reduction in active osteocalcin-expressing osteoblasts. In some embodiments, administration results in an increase of tartrate-resistant acid phosphatase (TRAP)-expression in osteoclasts. In some embodiments, administration results in an increase of C-terminal telopeptide of type I collagen (Ctx-I).

### BRIEF DESCRIPTION OF DRAWINGS

FIGS. 1A-1F show SHN3 expression and function in bone marrow-derived stromal cells (BSMCs) and Fibroblast-like synoviocytes (FLSs). FIG. 1A shows elevated levels of SHN3 mRNA in the serum of RA patients. FIG. 1B shows that three days after curdlan injection, total RNA was isolated from the serum, synovium, and tibia of 3-month-old female SKG mice. FIGS. 1C-1F show that after 4 hours of stimulation with TNF, IL-17A, or both TNF and IL-17A, total RNA was isolated from human BMSC, mouse BMSC, mouse COB and mouse FLS. SHN3 (HIVEP3) mRNA was measured by qPCR and normalized to HPRT mRNA. NS: no significance, *: P<0.05, **: P<0.005, ***: P.0.0005.

FIGS. 2A-2E show upregulated expression of SHN3 (HIVEP3) in synovium of RA patients. FIGS. 2A-2B show whole tissue transcriptome analysis of synovium from untreated early RA patients. Correlation with ultrasound synovial thickness score at joint biopsy (FIG. 2A) and correlation with joint swelling (FIG. 2B). FIG. 2C shows T cells, B cells, fibroblast, and monocyte were FACS (fluorescence-activated Cell Sorting) sorted from the synovium of human patients with leukocyte-rich or leukocyte-poor RA or healthy synovium, which were subjected for whole transcriptome analysis. FIGS. 2D-2E show single cell transcriptome analysis shows SHN3 expression in synovium of RA patients. SC-F: CD45- Podoplanin⁺sublining fibroblasts (F1: CD34+, F2: HLA+, F3: DKK3+, F4: CD55+), SC-M: CD45+CD14+Monocytes (M1: IL1B+, M2: NUPR1+, M3: C1QA+, M4: IFN-activated).

FIGS. 3A-3E show activation of the NF-κB pathway suppresses osteoblast differentiation (OBD) via SHN3 expression. FIG. 3A shows human BMSCs were treated with the indicated inhibitors prior to 4 hr stimulation with TNF plus IL-17A. FIG. 3B shows a constitutively active mutant of IKK (IKK-CA) upregulates SHN3 expression in osteoblasts. Calvarial osteoblasts (COBs) obtained from IKK-CA floxed mice were infected with lentivirus expressing vector control (WT) or CRE recombinase (IKK-CA), and cultured under undifferentiated (UD) or osteogenic (OBD) conditions for 6 days. shn3 mRNA was measured by qPCR and normalized to hprt mRNA. FIG. 3C shows WT and IKK-CA COBs and human BMSCs expressing vector or SHN3 were cultured under osteogenic conditions for 14 days. Mineralization activity was measured by alizarin red staining. FIG. 3D shows MicroCT analysis showing trabecular bone volume per total volume (Tra. BV/TV) in the femur of 2-month-old female mice (n=5˜6/group).

FIG. 3E shows a diagram of the promoter region of mouse hivep3 (shn3) gene. TSS, transcription start site; iTSS, internal TSS.

FIGS. 4A-4D show SHN3-deficiency did not affect both local and systemic inflammation in the SKG mouse model (shn3^{-/-} SKG). FIGS. 4A-4D show histology in the inflamed ankles (FIG. 4B) was assessed for determining infiltration of immune cells 7 weeks after curdlan treatment (i.p. injection). Synovial cells in inflamed joints (FIG. 4C) and splenocytes (FIG. 4D) were isolated and were subjected to flow cytometry analysis (n=5/group).

FIGS. 5A-5I show SHN3-deficiency prevents bone loss in the SKG mouse model. FIG. 5Ashows 3- month-old female mice (wild type, SKG, SHN3^{-/-}, or SKG:SHN3^{-/-}) were treated with curdlan (i.p. injection) and 7 weeks later, trabecular bone volume per total volume (BV/TV) in the femurs was assessed by microCT. 3D reconstruction images and relative quantification were displayed (n=6/group). FIGS. 5B-5C show immunohistochemistry analysis for osteocalcin demonstrating active OBs on the surface of trabecular bone (FIG. 5B) and relative quantification was displayed (FIG. 5C). Arrows indicate osteocalcin-expressing OBs. (n=3˜5/group) (FIG. 5B). FIGS. 5G-5I show MicroCT (FIG. 5G) and histology (FIGS. 5H-5I) analyses using hematoxylin and eosin (H&E) and TRAP staining. Protection from bone erosion, infiltration of immune cells, and OCs within the inflamed ankles of SKG and SKG:SHN3^{-/-} mice (n=6/group) were observed. Arrows (FIG. 5G) and stars (FIG. 5H, left) indicate focal bone erosion areas and infiltrated immune cells, respectively. NS: no significance, **: P≤0.01

FIGS. 6A-6Dshow SHN3-deficient OBs prevent bone loss in the K/BxN serum transfer (STA) model of RA. FIG. 6A show 2-month-old female SHN3 (f/f) and SHN3 (f/f);prx1 mice were treated with K/BxN serum (i.p. injection) and clinical joint inflammation was daily measured (n=5). Serum transfer arthritis clinical scoring (average clinical inflammation score and change in ankle thickness) was assessed. FIGS. 6B-6C show 10 days after serum injection, histologic analysis of joint inflammation and articular bone erosion in the ankle demonstrated no difference in inflammation in SHN3 (f/f);prx1 mice compared with SHN3 (f/f) mice, but a significant protection from joint erosion in SHN3 (f/f);prx1 mice was observed. FIG. 6D shows histologic analysis of pannus formation in the ankle demonstrated a decrease in TRAP+OCs (top). Alternatively, immunohistochemistry for osteocalcin in the pannus demonstrated a significant increase in and OCN+Obs (bottom).

FIG. 7 shows SHN3-deficiency protects TNF+IL-17A-induced suppression of OB differentiation. FIGS. 7A-7B show human BMSCs were infected with lentivirus (vector alone (control), SHN3 overepxression (SHN3), control-shRNA (Sh-con), or SHN3 knockdown (Sh-SHN3)) and cultured under osteogenic conditions for 14 days (FIG. 7A). FIG. 7B shows BMSCs expressing Sh-con or Sh-SHN3 were cultured in the absence or presence of TNF and IL-17A for 14 days. Mineralization activity was measured by alizarin red staining. FIG. 7C shows mouse COB s isolated form P5 WT and SHN3-KO pups were cultured in the absence or presence of TNF and IL-17A. 14 days after the culture, osteogenic marker gene expression (Tnalp mRNA levels and ibsp mRNA levels) was measured by RT-PCR and 21 days after the culture, mineralization activity was measured by alizarin red staining. ns: not significant, *: P<0.05, **: P≤0.01, ***: P≤0.001, ****: P≤0.0001.

FIGS. 8A-8D show conditional deletion of SHN3 in OB lineage cells prevents TNF-induced bone loss in mice. SHN3-floxed mice (SHN3^{fl/fl}) were crossed with Prx1-cre mice to delete SHN3 in limb-specific mesenchyme (SHN3^{prx1}), and these mice were further crossed with the mice expressing human TNF-α (TNFtg). FIGS. 8A and 8B (microCT analysis) show that articular bone erosion in the knee joints (FIG. 8A) and ankles (FIG. 8B) were protected in SHN3 (f/f);prx1; TNFtg mice while TNFtg mice showed severe articular bone erosion, often debilitating by 15 weeks of age. FIG. 8C (histologic analysis) shows that bone erosion in the ankles was markedly reduced in the absence of SHN3 while joint inflammation was comparable between TNFtg and SHN3 (f/f);prx1; TNFtg mice.

FIGS. 9A-9I show ERK-mediated phosphorylation of SHN3 downstream of TNF. FIGS. 9A-9B show C3H10T1/2 cells expressing Flag-SHN3 were stimulated with 20 μg/ml of TNF at the indicated time points, immunoprecipitated with anti-Flag (M2) beads, and immunoblotted with anti-phospho-serine/threonine Ab (FIG. 9A). Alternatively, cells were treated with MAPK inhibitors prior to TNF stimulation (FIG. 9B). FIG. 9C shows in vitro kinase assay showing recombinant ERK2 (rERKs)-induced phosphorylation of recombinant SHN3 (rSHN3). FIG. 9D shows a diagram showing functional domains and ERK-binding site in the BAS domain (white line) and five phosphorylation sites mediated by ERK (5810/5811/T851/5911/5913) in mouse SHN3. FIG. 9E shows an in vitro kinase assay showing ERK-mediated phosphorylation of recombinant SHN3 using wildtype rSHN3 (SHN3-WT) or rSHN3 mutants (SHN3-3KA, SHN3-5STA). FIG. 9F and FIG. 9G show wildtype SHN3 or SHN3 mutants were expressed in wildtype and SHN-KO BMSCs via lentivirus-mediated delivery and cultured under osteogenic conditions. FIG. 9H shows a diagram demonstrating substitution of three lysines to alanines in the endogenous Shn3 locus. FIG. 9I shows femoral bone mass of 2-month-old female mice was assessed by microCT (n=6˜8/group). Tb.BV/TV: trabecular bone volume per total volume, Tb. Th: trabecular thickness, Tb. N: trabecular number, Conn.Dn: connective density.

FIGS. 10A-10G show AAV-mediated silencing of SHN3 prevents bone loss in the SKG mouse model. FIG. 10A shows 3-month-old female WT and SKG mice were i.v. injected with the bone-targeting DSS.rAAV9 carrying EGFP two weeks prior to curdlan injection, and three weeks later, AAV's transduction in the cryosectioned femur and inflamed ankles was assessed by fluorescence microscopy. FIGS. 10B-G show 3-month-old female WT and SKG mice were i.v. injected with the bone-targeting DSS.rAAV9 carrying amiR-ctrl or amiR-shn3 two weeks prior to curdlan injection. shn3 mRNA levels in the tibia were assessed by qPCR (n=8/group) (FIG. 10B). FIG. 10C shows joint inflammation was measured weekly, showing that local inflammation was not altered by DSS.rAAV9 (n=5-6/group) FIGS. 10D-10E show flow cytometry analysis of splenocytes, showing that systemic inflammation was not altered by DSS.rAAV9 (n=1˜5/group). FIGS. 10F-10G show trabecular bone mass in the femur and articular bone erosion in inflamed ankles were assessed by MicroCT analysis

FIGS. 11A-11C show bone-targeting AAV-mediated silencing of cathepsin K (CTSK) prevents bone loss in the SKG mouse model. 3-month-old female WT and SKG mice were i.v. injected with the bone-targeting DSS.rAAV9 carrying amiR-ctrl or amiR-CTSK two weeks prior to curdlan injection (n=5˜8/group). FIG. 11A shows trabecular bone mass and cortical bone thickness in the femur were measured by MicroCT analysis. Representative 3D-reconstruction (left) and relative quantifications (right) are displayed. FIG. 11B shows representative 3D-reconstruction of foot by MicroCT is displayed. Arrows indicate focal bone erosions on ankles and feet. FIG. 11C shows joint inflammation was measured weekly, showing that inflammation is not altered by treatment with DSS.rAAV9 vectors (n=5˜8/group).

FIG. 12 shows a schematic diagram showing AAV vector construction. The AAV genome vector constructs will be packaged into the bone-targeting rAAV9 capsid (DSS.rAAV9).

FIGS. 13A-13D show generation of an engineered rAAV9 with tissue-specific miRNA-mediated repression of a transgene. FIGS. 13A-13B show single dose of 4×10¹¹ GCs of rAAV vectors carrying egfp transgene was i.v. injected into 2-month-old male mice, and 2 weeks later, EGFP expression in whole body (FIG. 13A) and individual tissues (FIG. 13B) was assessed by IVIS-100 optical imaging. FIG. 13C shows EGFP mRNA levels in total tissue RNA were measured by qPCR. FIG. 13D show EGFP expression in the cryo-sectioned femur was assessed by fluorescence microscopy (n=3/group, scale bars: 200 μm).

FIGS. 14A-14B show generation of inflammation-responsive rAAV9 vectors. FIG. 14A shows PB2 promoter-driven EGFP expression cassette was cloned into the AAV genome vector (PB2-GFP). Red: NF-κB-binding sites. Blue: Minimal FosP site. Bold: Restriction enzyme sites. (SEQ ID NO: 3). FIG. 14B shows 1 day after transfection with the vectors in the absence or presence of CD4/TLR4 plasmid, HEK293 cells were stimulated the indicated proinflammatory cytokines. EGFP expression was visualized by fluorescence microscopy.

FIGS. 15A-15B show expression of EGFP by pro-inflammatory cytokines in PB2-egfp expressing OBs and OCs. Calvarial osteoblasts (COB) or bone marrow-derived osteoclasts (BM-OC) were incubated with PBS (none) or rAAV9.PB2-egfp for 2 days. One day after treatment with the indicated pro-inflammatory cytokines, EGFP expression was assessed by fluorescence microscopy (FIG. 15A) and immunoblotting with anti-GFP antibody (FIG. 15B). The anti-Hsp90 antibody was used as a loading control. scale bars: 1 mm.

FIGS. 16A-16C show PB2-egfp-driven EGFP expression in the SKG mouse model of inflammatory arthritis. FIG. 16A shows a single dose of PBS or 4×10¹¹ GCs of rAAV9.PB2-egfp was i.v. injected into 2-month-old male mice and 2 weeks later, mice were treated with PBS or curdlan via i.p. injection. 3 weeks later, EGFP expression in whole body (left) and individual tissues (right) was assessed by IVIS-100 optical imaging. FIG. 16B shows EGFP mRNA levels in total tissue RNA were measured by qPCR. FIG. 16C shows EGFP expression in the cryo-sectioned tissues were assessed by fluorescence microscopy (scale bars: 400 μm).

FIG. 17 shows an experimental design for mouse models of RA.

FIG. 18 shows a schematic diagram showing one embodiment of molecular mechanisms of therapeutic AAV vectors described by the disclosure.

FIG. 19 show schematic depicting embodiments of gene expression constructs (e.g., rAAV vectors) that include CB promoter or OC-specific or OB-specific promoters.

FIG. 20 shows OCN promoter was specific to express GFP protein in mature osteoblasts while CB and RANK promoter express GFP protein in both mature osteoblasts and osteoclasts. 2 days after treatment of Calvarial osteoblasts (COB) or BM-OC with PBS (none) or rAAV9 with GFP expression driven by the CB, OCN, RANK promoter, COB and BM-OC were cultured under osteogenic and osteoclastogenic conditions, respectively. EGFP expression was assessed by fluorescence microscopy.

FIG. 21 show schematic depicting embodiments of gene expression constructs (e.g., rAAV vectors) that include combinations of ami-RNAs targeting one or more of the following: SHN3, CTSK, RANKL, and SOST.

### DETAILED DESCRIPTION

Aspects of the disclosure relate to methods and compositions for treating certain inflammatory conditions, for example rheumatoid arthritis (RA). The disclosure is based, in part, on compositions (e.g., isolated nucleic acids, vectors, rAAVs, etc.) that inhibit inflammation (e.g., reduce the effects of inflammatory cytokines) and/or reduce bone loss due to inflammation in a subject. In some embodiments, the compositions comprise one or more inhibitory nucleic acids that specifically target osteoclasts (OCs) or osteoblasts (OB s). In some embodiments, the compositions comprise one or more therapeutic proteins that inhibit cytokines. In some aspects, the disclosure relates to methods of treating inflammatory conditions, such as RA, using compositions described herein.

Isolated Nucleic Acids

Compositions and methods for delivering a transgene (e.g. an inhibitory RNA, such as an shRNA, miRNA, etc.) to a subject are provided in the disclosure. The compositions typically comprise an isolated nucleic acid encoding a transgene (e.g., a protein, an inhibitory nucleic acid, etc.) capable of modulating bone metabolism. For example, in some embodiments, a transgene reduces expression of a target protein, such as a target protein associated with promoting or inhibiting bone formation. In some embodiments, a transgene reduces expression or activity of a target protein, such as a target protein associated with promoting inflammation (e.g., pro-inflammatory cytokines, etc.).

“Bone metabolism” generally refers to a biological process involving bone formation and/or bone resorption. In some embodiments, bone metabolism involves the formation of new bone as produced by osteoblasts (OBs) and terminally-differentiated osteocytes, and/or mature bone tissue being resorbed by osteoclasts (OCs). OBs arise from the bone marrow derived mesenchymal/stromal cells that ultimately differentiate terminally into osteocytes. OB (and osteocyte) functions or activities include but are not limited to bone formation, bone mineralization, and regulation of OC activity. Decreased bone mass has been observed to result from inhibition of OB and/or osteocyte function or activity. Increased bone mass has been observed to result from increased OB and/or osteocyte function or activity. OCs arise from bone marrow-derived monocytes and in some embodiments have been observed to be controlled by signals from OBs and/or inflammation. OC functions include bone resorption. In some embodiments, decreased bone mass has been observed to result from increased OC activity. In some embodiments, increased bone mass has been observed to result from inhibition of OC activity. The disclosure is based, in part, on the recognition that inhibitory nucleic acids targeting certain OB- or OC-expressed proteins (e.g., SHN3, SOST, CTSK, RANK, RANKL, etc.) are capable of reducing inflammation-induced bone loss in a subject even in the presence of pro-inflammatory cytokines. Compositions encoding gene products that modulate bone metabolism are described, for example, in PCT Publication Number WO2019/183605, the entire contents of which are incorporated herein by reference.

In some embodiments, an isolated nucleic acid encodes one or more (e.g., 1, 2, 3, 4, 5, 6, 7, 8, 9, 10, or more) inhibitory nucleic acids, for example dsRNA, siRNA, shRNA, miRNA, artificial microRNA (ami-RNA), etc.). Generally, an inhibitory nucleic acid specifically binds to (e.g., hybridizes with) at least two (e.g., 2, 3, 4, 5, 6, 7, 8, 9, 10, 11, 12, 13, 14, 15, 16, 17, 18, 19, 20, 21, 22, 23, 24, 25, or more) continuous bases of a gene encoding a gene product (e.g., a protein) associated with bone metabolism (e.g., SOST, SHN3, CTSK, RANK, RANKL, etc.). As used herein “continuous bases” refers to two or more nucleotide bases that are covalently bound (e.g., by one or more phosphodiester bond, etc.) to each other (e.g. as part of a nucleic acid molecule). In some embodiments, the at least one inhibitory nucleic acid is about 50%, about 60% about 70% about 80% about 90%, about 95%, about 99% or about 100% identical to the two or more (e.g., 2, 3, 4, 5, 6, 7, 8, 9, 10, 11, 12, 13, 14, 15, 16, 17, 18, 19, 20, 21, 22, 23, 24, 25, or more) continuous nucleotide bases of a gene encoding a gene product (e.g., a protein) associated with bone metabolism (e.g., SOST, SHN3, CTSK, RANK, RANKL, etc.).

A “microRNA” or “miRNA” is a small non-coding RNA molecule capable of mediating transcriptional or post-translational gene silencing. Typically, miRNA is transcribed as a hairpin or stem-loop (e.g., having a self-complementarity, single-stranded backbone) duplex structure, referred to as a primary miRNA (pri-miRNA), which is enzymatically processed (e.g., by Drosha, DGCR8, Pasha, etc.) into a pre-miRNA. The length of a pri-miRNA can vary. In some embodiments, a pri-miRNA ranges from about 100 to about 5000 base pairs (e.g., about 100, about 200, about 500, about 1000, about 1200, about 1500, about 1800, or about 2000 base pairs) in length. In some embodiments, a pri-miRNA is greater than about 200 base pairs in length (e.g., 2500, 5000, 7000, 9000, or more base pairs in length).

Pre-miRNA, which is also characterized by a hairpin or stem-loop duplex structure, can also vary in length. In some embodiments, pre-miRNA ranges in size from about 40 base pairs in length to about 500 base pairs in length. In some embodiments, pre-miRNA ranges in size from about 50 to 100 base pairs in length. In some embodiments, pre-miRNA ranges in size from about 50 to about 90 base pairs in length (e.g., about 50, about 52, about 54, about 56, about 58, about 60, about 62, about 64, about 66, about 68, about 70, about 72, about 74, about 76, about 78, about 80, about 82, about 84, about 86, about 88, or about 90 base pairs in length).

Generally, pre-miRNA is exported into the cytoplasm, and enzymatically processed by Dicer to first produce an imperfect miRNA/miRNA* duplex and then a single-stranded mature miRNA molecule, which is subsequently loaded into the RNA-induced silencing complex (RISC). Typically, a mature miRNA molecule ranges in size from about 19 to about 30 base pairs in length. In some embodiments, a mature miRNA molecule is about 19, about 20, about 21, about 22, about 23, about 24, about 25, about 26, about 27, about 28, about 29, or about 30 base pairs in length.

In some aspects, the disclosure provides isolated nucleic acids and vectors (e.g., rAAV vectors) that encode one or more artificial miRNAs. As used herein “artificial miRNA” or “ami-RNA” refers to an endogenous pri-miRNA or pre-miRNA (e.g., a miRNA backbone, which is a precursor miRNA capable of producing a functional mature miRNA), in which the miRNA and miRNA* (e.g., passenger strand of the miRNA duplex) sequences have been replaced with corresponding ami-RNA/ami-RNA* sequences that direct highly efficient RNA silencing of the targeted gene, for example as described by Eamens et al. (2014), Methods Mol. Biol. 1062:211-224. For example, in some embodiments an artificial miRNA comprises a miR-155 pri-miRNA backbone into which a sequence encoding a bone metabolism modulating (e.g., bone formation inhibiting agent) miRNA has been inserted in place of the endogenous miR-155 mature miRNA-encoding sequence. In some embodiments, mouse or human miRNA (e.g., an artificial miRNA) as described by the disclosure comprises a miR-155 backbone sequence, a miR-33 backbone sequence, a miR-30 backbone sequence, a mir-64 backbone sequence, or a miR-122 backbone sequence.

In some embodiments, the present disclosure provides an isolated nucleic acid comprising a transgene encoding an inhibitory nucleic acid (e.g., an artificial microRNA) targeting the SHN3 gene (GeneID: 59269), which encodes the Schnurri-3 protein. The Schnurri-3 (SHN3) protein is a transcription factor that regulates NK-κβ(3 protein expression and immunoglobulin and T-cell receptor antibody recombination. In some embodiments, the SHN3 gene is represented by the NCBI Accession Number NM_001127714.2 or NM_024503.5. In some embodiments, the SHN3 protein is represented by the NCBI Accession Number NP_001121186.1 or NP_078779.2.

In some aspects, the disclosure relates to an isolated nucleic acid comprising a transgene encoding an artificial microRNA is used to reduce SHN3 expression (e.g., expression of one or more gene products from an SHN3 gene, for example an mRNA encoded by SHN3 gene.

In some embodiments, an artificial microRNA targets (e.g., binds to, or comprises a region of complementarity with) at least 6 continuous nucleotides of a SHN3 gene (e.g., an mRNA transcript transcribed from a SHN3 gene). In some embodiments, an artificial microRNA targets (e.g., binds to, or comprises a region of complementarity with) between 6 and continuous nucleotides of a SHN3 gene (e.g., an mRNA transcript transcribed from a SHN3 gene). In some embodiments, an artificial microRNA targets between 12-24 continuous nucleotides of a SHN3 gene (e.g., an mRNA transcript transcribed from a SHN3 gene). In some embodiments, an artificial microRNA targets between 9-27 continuous nucleotides of the SHN3 gene (e.g., an mRNA transcript transcribed from a SHN3 gene). In some embodiments, an artificial microRNA targets at least 6, 7, 8, 9, 10, 11, 12, 13, 14, 15, 16, 17, 18, 19, 20, 21, 22, 23, 24, 25, 26, 27, 28, 29, or 30 continuous nucleotides of a SHN3 gene (e.g., an mRNA transcript transcribed from a SHN3 gene).

In some embodiments, the present disclosure provides an isolated nucleic acid comprising a transgene encoding an inhibitory nucleic acid (e.g., an artificial microRNA) targeting the CTSK gene (GeneID: 1513), which encodes the Cathepsin K protein. The Cathepsin K (CTSK) protein is a lysosomal cysteine protease involved in bone remodeling and resorption. In some embodiments, the CTSK gene is represented by the NCBI Accession Number NM_000396. In some embodiments, the CTSK protein is represented by the NCBI Accession Number NP_000387.

In some aspects, the disclosure relates to an isolated nucleic acid comprising a transgene encoding an artificial microRNA is used to reduce CTSK expression (e.g., expression of one or more gene products from an CTSK gene, for example an mRNA encoded by a CTSK gene.

In some embodiments, an artificial microRNA targets (e.g., binds to, or comprises a region of complementarity with) at least 6 continuous nucleotides of a CTSK gene (e.g., an mRNA transcript transcribed from a CTSK gene). In some embodiments, an artificial microRNA targets (e.g., binds to, or comprises a region of complementarity with) between 6 and continuous nucleotides of a CTSK gene (e.g., an mRNA transcript transcribed from a CTSK gene). In some embodiments, an artificial microRNA targets between 12-24 continuous nucleotides of a CTSK gene (e.g., an mRNA transcript transcribed from a CTSK gene). In some embodiments, an artificial microRNA targets between 9-27 continuous nucleotides of the CTSK gene (e.g., an mRNA transcript transcribed from a CTSK gene). In some embodiments, an artificial microRNA targets at least 6, 7, 8, 9, 10, 11, 12, 13, 14, 15, 16, 17, 18, 19, 20, 21, 22, 23, 24, 25, 26, 27, 28, 29, or 30 continuous nucleotides of a CTSK gene (e.g., an mRNA transcript transcribed from a CTSK gene).

In some embodiments, the present disclosure provides an isolated nucleic acid comprising a transgene encoding an inhibitory nucleic acid (e.g., an artificial microRNA) targeting the SOST gene (GeneID: 50964), which encodes the sclerostin protein. The sclerostin protein is a secreted glycoprotein that antagonizes bone morphogenic protein (BMP). In some embodiments, the SOST gene is represented by the NCBI Accession Number NM_025237. In some embodiments, the SOST protein is represented by the NCBI Accession Number NP_079513.

In some aspects, the disclosure relates to an isolated nucleic acid comprising a transgene encoding an artificial microRNA is used to reduce SOST expression (e.g., expression of one or more gene products from an SOST gene, for example an mRNA encoded by a SOST gene. In some embodiments, an artificial microRNA targets (e.g., binds to, or comprises a region of complementarity with) at least 6 continuous nucleotides of a SOST gene (e.g., an mRNA transcript transcribed from a SOST gene). In some embodiments, an artificial microRNA targets (e.g., binds to, or comprises a region of complementarity with) between 6 and 30 continuous nucleotides of a SOST gene (e.g., an mRNA transcript transcribed from a SOST gene). In some embodiments, an artificial microRNA targets between 12-24 continuous nucleotides of a SOST gene (e.g., an mRNA transcript transcribed from a SOST gene). In some embodiments, an artificial microRNA targets between 9-27 continuous nucleotides of the SOST gene (e.g., an mRNA transcript transcribed from a CTSK gene). In some embodiments, an artificial microRNA targets at least 6, 7, 8, 9, 10, 11, 12, 13, 14, 15, 16, 17, 18, 19, 20, 21, 22, 23, 24, 25, 26, 27, 28, 29, or 30 continuous nucleotides of a SOST gene (e.g., an mRNA transcript transcribed from a SOST gene).

In some embodiments, the present disclosure provides an isolated nucleic acid comprising a transgene encoding an artificial microRNA targeting the RANKL gene (GeneID: 8600), also referred to as tumor necrosis factor ligand superfamily member 11 (TNFSF11), which encodes the RANKL protein. The RANKL protein is a type II membrane protein that plays a role in controlling bone regeneration and remodeling. In some embodiments, the RANKL gene is represented by the NCBI Accession Number NM_003701 or NM_033012. In some embodiments, the RANKL protein is represented by the NCBI Accession Number NP_003692 or NP_143026.

In some aspects, the disclosure relates to an isolated nucleic acid comprising a transgene encoding an artificial microRNA is used to reduce RANKL expression (e.g., expression of one or more gene products from an RANKL gene, for example an mRNA encoded by a RANKL gene).

In some embodiments, an artificial microRNA targets (e.g., binds to, or comprises a region of complementarity with) at least 6 continuous nucleotides of a RANKL gene (e.g., an mRNA transcript transcribed from a RANKL gene). In some embodiments, an artificial microRNA targets (e.g., binds to, or comprises a region of complementarity with) between 6 and continuous nucleotides of a RANKL gene (e.g., an mRNA transcript transcribed from a RANKL gene). In some embodiments, an artificial microRNA targets between 12-24 continuous nucleotides of a RANKL gene (e.g., an mRNA transcript transcribed from a RANKL gene). In some embodiments, an artificial microRNA targets between 9-27 continuous nucleotides of the RANKL gene (e.g., an mRNA transcript transcribed from a RANKL gene). In some embodiments, an artificial microRNA targets at least 6, 7, 8, 9, 10, 11, 12, 13, 14, 15, 16, 17, 18, 19, 20, 21, 22, 23, 24, 25, 26, 27, 28, 29, or 30 continuous nucleotides of a RANKL gene (e.g., an mRNA transcript transcribed from a RANKL gene).

In some embodiments, the present disclosure provides an isolated nucleic acid comprising a transgene encoding an artificial microRNA targeting the RANK gene (GeneID: 21934), also referred to as tumor necrosis factor ligand superfamily member 11 (TNFRSF11A), which encodes the RANK protein. The RANK protein is essential for RANKL-induced osteoclastogenesis and also involved in the regulation of interactions between T cells and dendritic cells. In some embodiments, the RANK gene is represented by the NCBI Accession Number NM_009399.3. In some embodiments, the RANK protein is represented by the NCBI Accession Number NP_033425.3.

In some aspects, the disclosure relates to an isolated nucleic acid comprising a transgene encoding an artificial microRNA is used to reduce RANK expression (e.g., expression of one or more gene products from a RANK gene, for example an mRNA encoded by a RANK gene.

In some embodiments, an artificial microRNA targets (e.g., binds to, or comprises a region of complementarity with) at least 6 continuous nucleotides of a RANK gene (e.g., an mRNA transcript transcribed from a RANK gene). In some embodiments, an artificial microRNA targets (e.g., binds to, or comprises a region of complementarity with) between 6 and continuous nucleotides of a RANK gene (e.g., an mRNA transcript transcribed from a RANK gene). In some embodiments, an artificial microRNA targets between 12-24 continuous nucleotides of a RANK gene (e.g., an mRNA transcript transcribed from a RANK gene). In some embodiments, an artificial microRNA targets between 9-27 continuous nucleotides of the RANK gene (e.g., an mRNA transcript transcribed from a RANK gene). In some embodiments, an artificial microRNA targets at least 6, 7, 8, 9, 10, 11, 12, 13, 14, 15, 16, 17, 18, 19, 20, 21, 22, 23, 24, 25, 26, 27, 28, 29, or 30 continuous nucleotides of a RANK gene (e.g., an mRNA transcript transcribed from a RANK gene).

In some embodiments, the disclosure relates to an isolated nucleic acid comprising a transgene encoding a first inhibitory nucleic acid targeting a gene selected from SHN3, CTSK, SOST, RANK, and RANKL and a second inhibitory nucleic acid targeting a gene selected from SHN3, CTSK, SOST, RANK, and RANKL. For example, the first inhibitory nucleic acid can target SHN3 and the second inhibitory nucleic acid can target RANK. In another example, the first inhibitory nucleic acid can target SHN3 and the second inhibitory nucleic acid can target SHN3. In another example, the first inhibitory nucleic acid can target SOST and the second inhibitory nucleic acid can target RANKL.

In some embodiments, an artificial microRNA is between 6-50 nucleotides in length. In some embodiments, an artificial microRNA is between 8-24 nucleotides in length. In some embodiments, an artificial microRNA is between 12-36 nucleotides in length. In some embodiments, an artificial microRNA is 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length.

In some embodiments, an isolated inhibitory nucleic acid decreases expression of a target gene by between 50% and 99% (e.g., any integer between 50% and 99%, inclusive) compared to a control. In some aspects, an isolated inhibitory nucleic acid decreases expression of a target gene by between 75% and 90% compared to a control. In some aspects, an isolated inhibitory nucleic acid decreases expression of a target gene by between 80% and 99% compared to a control.

In some embodiments, an isolated inhibitory nucleic acid decreases expression of a SHN3 gene by between 50% and 99% (e.g., any integer between 50% and 99%, inclusive) compared to a control. In some embodiments, an isolated inhibitory nucleic acid decreases expression of a SHN3 gene by between 75% and 90% compared to a control. In some aspects, an isolated inhibitory nucleic acid decreases expression of a SHN3 gene by between 80% and 99% compared to a control.

In some embodiments, the decreased expression of the SHN3 gene prevents or reverses a systemic bone loss. In some embodiments, systemic bone loss is osteoporosis. In some embodiments, systemic bone loss can be any bone losses that impact more than regional bone health. In some embodiments, the decreased expression of the SHN3 gene prevents osteoblasts from inflammation. In some embodiments, the decreased expression of the SHN3 gene suppresses inflammation-induced activation of osteoclasts. In some embodiments, the decreased expression of the SHN3 gene prevents inflammation-induced bone loss.

In some embodiments, the decreased expression of the SHN3 gene prevents or reverses a regional bone loss. In some embodiments, the regional bone loss can be any bone losses that impact regional body parts of a subject. In some embodiments, the regional body parts can be ankles, wrists, knees, and/or limbs.

In some embodiments, the decreased expression of the SHN3 gene reduces the levels of tartrate-resistant acid phosphatase (TRAP). In some embodiments, the decreased expression of the SHN3 gene reduces the levels of TRAP by between 50% and 99% (e.g., any integer between 50% and 99%, inclusive) compared to a control. In some embodiments, the decreased expression of the SHN3 gene reduces the levels of TRAP by between 75% and 90% compared to a control. In some embodiments, the decreased expression of the SHN3 gene reduces the levels of TRAP by between 80% and 99% compared to a control.

In some embodiments, the decreased expression of the SHN3 gene increases the levels of the tartrate-resistant acid phosphatase (TRAP)-expression osteoclasts. In some embodiments, the decreased expression of the SHN3 gene increases the levels of the tartrate-resistant acid phosphatase (TRAP)-expression osteoclasts by between 50% and 99% (e.g., any integer between 50% and 99%, inclusive) compared to a control. In some embodiments, the decreased expression of the SHN3 gene increases the levels of TRAP-expression osteoclasts by between 75% and 90% compared to a control. In some embodiments, the decreased expression of the SHN3 gene increases the levels of TRAP-expression osteoclasts by between 80% and 99% compared to a control.

In some embodiments, an isolated inhibitory nucleic acid decreases expression of a CTSK gene by between 50% and 99% (e.g., any integer between 50% and 99%, inclusive) compared to a control. In some embodiments, an isolated inhibitory nucleic acid decreases expression of a CTSK gene by between 75% and 90% compared to a control. In some aspects, an isolated inhibitory nucleic acid decreases expression of a CTSK gene by between 80% and 99% compared to a control.

In some embodiments, an isolated inhibitory nucleic acid decreases expression of a SOST gene by between 50% and 99% (e.g., any integer between 50% and 99%, inclusive) compared to a control. In some embodiments, an isolated inhibitory nucleic acid decreases expression of a SOST gene by between 75% and 90% compared to a control. In some aspects, an isolated inhibitory nucleic acid decreases expression of a SOST gene by between 80% and 99% compared to a control.

In some embodiments, an isolated inhibitory nucleic acid decreases expression of a RANKL gene by between 50% and 99% (e.g., any integer between 50% and 99%, inclusive) compared to a control. In some embodiments, an isolated inhibitory nucleic acid decreases expression of a RANKL gene by between 75% and 90% compared to a control. In some aspects, an isolated inhibitory nucleic acid decreases expression of a RANKL gene by between 80% and 99% compared to a control.

In some embodiments, an isolated inhibitory nucleic acid decreases expression of a RANK gene by between 50% and 99% (e.g., any integer between 50% and 99%, inclusive) compared to a control. In some embodiments, an isolated inhibitory nucleic acid decreases expression of a RANK gene by between 75% and 90% compared to a control. In some aspects, an isolated inhibitory nucleic acid decreases expression of a RANK gene by between 80% and 99% compared to a control.

In some embodiments, an isolated inhibitory nucleic acid decreases levels of C-terminal telopeptide of type I collagen (Ctx-I) by between 50% and 99% (e.g., any integer between 50% and 99%, inclusive) compared to a control. In some embodiments, an isolated inhibitory nucleic acid decreases levels of Ctx-I by between 75% and 90% compared to a control. In some aspects, an isolated inhibitory nucleic acid decreases levels of Ctx-I by between 80% and 99% compared to a control.

In some embodiments, an isolated inhibitory nucleic acid reduces bone loss by at least 5%, 10%, 20%, 30%, 40%, 50%, 60%, 70%, 80%, 90%, 100%, or by at least 2-fold, at least 5-fold, at least 10-fold, at least 20-fold, at least 50-fold, at least 100-fold, or at least 1000-fold compared to a control.

In some embodiments, an isolated inhibitory nucleic acid reduces active osteocalcin-expressing osteoblasts by at least 5%, 10%, 20%, 30%, 40%, 50%, 60%, 70%, 80%, 90%, 100%, or by at least 2-fold, at least 5-fold, at least 10-fold, at least 20-fold, at least 50-fold, at least 100-fold, or at least 1000-fold compared to a control.

As used herein, “control” can refer to any subjects who do not have, are not suspected of, or are at risk of developing a disease or disorder associated with dysregulated bone metabolism. “Control” can refer to the same subject before receiving the treatment disclosed herein. The control does not have one or more signs or symptoms of an inflammatory disease. The control can be a normal, healthy subject.

The disclosure is based, in part, on expression of certain inhibitors of pro-inflammatory cytokine activity (e.g., interleukin-1 (IL-1), IL-6, IL-12, IL-17a, IL-17f, IL-18, IL-22, IL-23, tumor necrosis factor alpha (TNF-α), interferon gamma (IFNγ), granulocyte-macrophage colony stimulating factor (GM-CSF), etc.) in cells expressing inhibitory nucleic acids described herein, results in increased inhibition of inflammation-induced bone loss. In some embodiments, transgenes described herein further encode one or more therapeutic proteins. In some embodiments, the one or more therapeutic proteins reduce expression or activity of pro-inflammatory cytokines in a subject. In some embodiments, the therapeutic protein is a soluble protein. In some embodiments, the soluble protein is soluble tumor necrosis factor alpha receptor 2 (sTNFR2) or soluble IL-1 receptor antagonist (sIL1Rα). In some embodiments, the soluble protein is soluble tumor necrosis factor alpha receptor 2 (sTNFR2) and soluble IL-1 receptor antagonist (sIL1Rα). In some embodiments, the soluble protein can be any protein that reduces expression or activity of pro-inflammatory cytokines in a subject.

In some embodiments, the present disclosure provides an isolated nucleic acid comprising a transgene encoding soluble tumor necrosis factor alpha (TNF-α) receptor 2 (sTNFR2). The sTNFR2 proteins are the circulating forms of their membrane bound. In some embodiments, the sTNFR2 gene is represented by the NCBI Accession Number NM_001065.4 or NM_001066.3. In some embodiments, the sTNFR2 protein is represented by the NCBI Accession Number NP_001056.1 or NP_001057.1.

In some embodiments, a sTNFR2 protein is encoded by the nucleic acid sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, or are 100% identical, including all values in between to SEQ ID NO: 6. In some embodiments, a sTNFR2 protein is encoded by the nucleic acid sequence set forth in SEQ ID NO: 6. In some embodiments, a sTNFR2 protein is encoded by the nucleic acid sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, or are 100% identical, including all values in between to SEQ ID NO: 7. In some embodiments, a sTNFR2 protein comprises the amino acid sequence set forth in SEQ ID NO: 7.

In some embodiments, the present disclosure provides an isolated nucleic acid comprising a transgene encoding soluble IL-1 receptor antagonist (sIL1Rα). The sIL1Rαproteins bind to IL-1 cell surface receptors and inhibit IL-1 signaling. In some embodiments, the sIL1Rαgene is represented by the NCBI Accession Number NM_000577, NM_173841, NM_173842, NM_173843, or NM_001318914. In some embodiments, the sIL1Rαprotein is represented by the NCBI Accession Number NP_000568, NP_001305843, NP_776213, NP_776214, or NP_776215.

In some embodiments, a sIL1Rαprotein is encoded by the nucleic acid sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, or are 100% identical, including all values in between to SEQ ID NO: 34. In some embodiments, a sIL1Rαprotein is encoded by the nucleic acid sequence set forth in SEQ ID NO: 34. In some embodiments, a sIL1Rαprotein is encoded by the nucleic acid sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, or are 100% identical, including all values in between to SEQ ID NO: 35. In some embodiments, a sIL1Rαprotein comprises the amino acid sequence set forth in SEQ ID NO: 35.

In some embodiments, expression of sTNFR2 and/or sIL1Rαdecreases expression or activity of pro-inflammatory cytokines (e.g., interleukin-1 (IL-1), IL-6, IL-12, IL-17a, IL-17f, IL-18, IL-22, IL-23, tumor necrosis factor alpha (TNF-α), interferon gamma (IFNγ), granulocyte-macrophage colony stimulating factor (GM-CSF), etc.) in a cell by between 50% and 99% (e.g., any integer between 50% and 99%, inclusive). In some aspects, expression of sTNFR2 and/or sIL1Rαdecreases expression or activity of pro-inflammatory cytokines (e.g., interleukin-1 (IL-1), IL-6, IL-12, IL-17a, IL-17f, IL-18, IL-22, IL-23, tumor necrosis factor alpha (TNF-α), interferon gamma (IFNγ), granulocyte-macrophage colony stimulating factor (GM-CSF), etc.) in a cell by between 75% and 90%. In some aspects, expression of sTNFR2 and/or sIL1Rαdecreases expression or activity of pro-inflammatory cytokines (e.g., interleukin-1 (IL-1), IL-6, IL-12, IL-17a, IL-17f, IL-18, IL-22, IL-23, tumor necrosis factor alpha (TNF-α), interferon gamma (IFNγ), granulocyte-macrophage colony stimulating factor (GM-CSF), etc.) in a cell by between 80% and 99%. In some embodiments, expression of sTNFR2 and/or sIL1Rαdecreases expression or activity of pro-inflammatory cytokines (e.g., interleukin-1 (IL-1), IL-6, IL-12, IL-17a, IL-17f, IL-18, IL-22, IL-23, tumor necrosis factor alpha (TNF-α), interferon gamma (IFNγ), granulocyte-macrophage colony stimulating factor (GM-CSF), etc.) in a cell by between 50% and 99% (e.g., any integer between 50% and 99%, inclusive). In some embodiments, expression of sTNFR2 and/or sIL1Rαdecreases expression or activity of pro-inflammatory cytokines (e.g., interleukin-1 (IL-1), IL-6, IL-12, IL-17a, IL-17f, IL-18, IL-22, IL-23, tumor necrosis factor alpha (TNF-α), interferon gamma (IFNγ), granulocyte-macrophage colony stimulating factor (GM-CSF) etc.) in a cell by between 75% and 90%. In some aspects, expression of sTNFR2 and/or sIL1Rαdecreases expression or activity of pro-inflammatory cytokines (e.g., interleukin-1 (IL-1), IL-6, IL-12, IL-17a, IL-17f, IL-18, IL-22, IL-23, tumor necrosis factor alpha (TNF-α), interferon gamma (IFNγ), granulocyte-macrophage colony stimulating factor (GM-CSF), etc.) in a cell by between 80% and 99%.

A region comprising a transgene (e.g., a second region, third region, fourth region, etc.) may be positioned at any suitable location of the isolated nucleic acid. The region may be positioned in any untranslated portion of the nucleic acid, including, for example, an intron, a 5′ or 3′ untranslated region, etc.

In some cases, it may be desirable to position the region (e.g., the second region, third region, fourth region, etc.) upstream of the first codon of a nucleic acid sequence encoding a protein (e.g., a protein coding sequence). For example, the region may be positioned between the first codon of a protein coding sequence) and 2000 nucleotides upstream of the first codon. The region may be positioned between the first codon of a protein coding sequence and 1000 nucleotides upstream of the first codon. The region may be positioned between the first codon of a protein coding sequence and 500 nucleotides upstream of the first codon. The region may be positioned between the first codon of a protein coding sequence and 250 nucleotides upstream of the first codon. The region may be positioned between the first codon of a protein coding sequence and 150 nucleotides upstream of the first codon.

In some cases (e.g., when a transgene lacks a protein coding sequence), it may be desirable to position the region (e.g., the second region, third region, fourth region, etc.) upstream of the poly-A tail of a transgene. For example, the region may be positioned between the first base of the poly-A tail and 2000 nucleotides upstream of the first base. The region may be positioned between the first base of the poly-A tail and 1000 nucleotides upstream of the first base. The region may be positioned between the first base of the poly-A tail and 500 nucleotides upstream of the first base. The region may be positioned between the first base of the poly-A tail and 250 nucleotides upstream of the first base. The region may be positioned between the first base of the poly-A tail and 150 nucleotides upstream of the first base. The region may be positioned between the first base of the poly-A tail and 100 nucleotides upstream of the first base. The region may be positioned between the first base of the poly-A tail and 50 nucleotides upstream of the first base. The region may be positioned between the first base of the poly-A tail and 20 nucleotides upstream of the first base. In some embodiments, the region is positioned between the last nucleotide base of a promoter sequence and the first nucleotide base of a poly-A tail sequence.

In some cases, the region may be positioned downstream of the last base of the poly-A tail of a transgene. The region may be between the last base of the poly-A tail and a position 2000 nucleotides downstream of the last base. The region may be between the last base of the poly-A tail and a position 1000 nucleotides downstream of the last base. The region may be between the last base of the poly-A tail and a position 500 nucleotides downstream of the last base. The region may be between the last base of the poly-A tail and a position 250 nucleotides downstream of the last base. The region may be between the last base of the poly-A tail and a position 150 nucleotides downstream of the last base. It should be appreciated that in cases where a transgene encodes more than one miRNA, each miRNA may be positioned in any suitable location within the transgene. For example, a nucleic acid encoding a first miRNA may be positioned in an intron of the transgene and a nucleic acid sequence encoding a second miRNA may be positioned in another untranslated region (e.g., between the last codon of a protein coding sequence and the first base of the poly-A tail of the transgene).

In some embodiments, the transgene further comprises a nucleic acid sequence encoding one or more expression control sequences (e.g., a promoter, etc.). Expression control sequences include appropriate transcription initiation, termination, promoter and enhancer sequences; efficient RNA processing signals such as splicing and polyadenylation (poly-A) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters which are native, constitutive, inducible and/or tissue-specific, are known in the art and may be utilized.

In some aspects, the disclosure relates to transgenes comprising one or more promoters that are capable of expressing gene product(s) in osteoblasts (OB s) or osteoclasts (OCs). A “promoter” refers to a DNA sequence recognized by the synthetic machinery of the cell, or introduced synthetic machinery, required to initiate the specific transcription of a gene. The phrases “operatively positioned,” “under control” or “under transcriptional control” means that the promoter is in the correct location and orientation in relation to the nucleic acid to control RNA polymerase initiation and expression of the gene.

For nucleic acids encoding proteins, a poly-A sequence generally is inserted following the transgene sequences and before the 3′ AAV ITR sequence. A rAAV construct useful in the present disclosure may also contain an intron, desirably located between the promoter/enhancer sequence and the transgene. One possible intron sequence is derived from SV-40, and is referred to as the SV-40 T intron sequence. Another vector element that may be used is an internal ribosome entry site (IRES). An IRES sequence is used to produce more than one polypeptide from a single gene transcript. An IRES sequence would be used to produce a protein that contains more than one polypeptide chains. Selection of these and other common vector elements are conventional, and many such sequences are available [see, e.g., Sambrook et al., and references cited therein at, for example, pages 3.18 3.26 and 16.17 16.27 and Ausubel et al., Current Protocols in Molecular Biology, John Wiley & Sons, New York, 1989]. In some embodiments, a Foot and Mouth Disease Virus 2A sequence is included in polyprotein; this is a small peptide (approximately 18 amino acids in length) that has been shown to mediate the cleavage of polyproteins (Ryan, M D et al., EMBO, 1994; 4: 928-933; Mattion, N M et al., J Virology, November 1996; p. 8124-8127; Furler, S et al., Gene Therapy, 2001; 8: 864-873; and Halpin, C et al., The Plant Journal, 1999; 4: 453-459). The cleavage activity of the 2A sequence has previously been demonstrated in artificial systems including plasmids and gene therapy vectors (AAV and retroviruses) (Ryan, M D et al., EMBO, 1994; 4: 928-933; Mattion, N M et al., J Virology, November 1996; p. 8124-8127; Furler, S et al., Gene Therapy, 2001; 8: 864-873; and Halpin, C et al., The Plant Journal, 1999; 4: 453-459; de Felipe, P et al., Gene Therapy, 1999; 6: 198-208; de Felipe, Petal., Human Gene Therapy, 2000; 11: 1921-1931.; and Klump, H et al., Gene Therapy, 2001; 8: 811-817).

Examples of constitutive promoters include, without limitation, the retroviral Rous sarcoma virus (RSV) LTR promoter (optionally with the RSV enhancer), the cytomegalovirus (CMV) promoter (optionally with the CMV enhancer) [see, e.g., Boshart et al., Cell, 41:521-530 (1985)], the SV40 promoter, the dihydrofolate reductase promoter, the β-actin promoter, the phosphoglycerol kinase (PGK) promoter, and the EF1α promoter [Invitrogen]. In some embodiments, a promoter is an enhanced chicken β-actin promoter. In some embodiments, a promoter is a U6 promoter.

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad. Many other systems have been described and can be readily selected by one of skill in the art. Examples of inducible promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system (WO 98/10088); the ecdysone insect promoter (No et al., Proc. Natl. Acad. Sci. USA, 93:3346-3351 (1996)), the tetracycline-repressible system (Gossen et al., Proc. Natl. Acad. Sci. USA, 89:5547-5551 (1992)), the tetracycline-inducible system (Gossen et al., Science, 268:1766-1769 (1995), see also Harvey et al., Curr. Opin. Chem. Biol., 2:512-518 (1998)), the RU486-inducible system (Wang et al., Nat. Biotech., 15:239-243 (1997) and Wang et al., Gene Ther., 4:432-441 (1997)) and the rapamycin-inducible system (Magari et al., J. Clin. Invest., 100:2865-2872 (1997)). Still other types of inducible promoters which may be useful in this context are those which are regulated by a specific physiological state, e.g., temperature, acute phase, a particular differentiation state of the cell, or in replicating cells only. In some embodiments, an inducible promoter is induced (e.g., activated transcriptionally) in the presence of inflammatory cytokines.

In some embodiments, an inflammation-induced promoter comprises a NF-kappa B (NFκB) promoter. In some embodiments, a NF-kappa B (NFκB) promoter is a PB2 promoter. In some embodiments, a NF-kappa B (NFκB) promoter (e.g., a PB2 promoter) comprises the nucleic acid sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, or are 100% identical, including all values in between to SEQ ID NO: 3. In some embodiments, a NF-kappa B (NFκB) promoter (e.g., a PB2 promoter) comprises the nucleic acid sequence set forth in SEQ ID NO: 3.

In another embodiment, the native promoter for the transgene will be used. The native promoter may be preferred when it is desired that expression of the transgene should mimic the native expression. The native promoter may be used when expression of the transgene must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

In some embodiments, the regulatory sequences impart tissue-specific gene expression capabilities. In some cases, the tissue-specific regulatory sequences bind tissue-specific transcription factors that induce transcription in a tissue specific manner. Such tissue-specific regulatory sequences (e.g., promoters, enhancers, etc.) are well known in the art. Exemplary tissue-specific regulatory sequences include, but are not limited to the following tissue specific promoters: a liver-specific thyroxin binding globulin (TBG) promoter, an insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a a-myosin heavy chain (a-MHC) promoter, or a cardiac Troponin T (cTnT) promoter. Other exemplary promoters include Beta-actin promoter, hepatitis B virus core promoter, Sandig et al., Gene Ther., 3:1002-9 (1996); alpha-fetoprotein (AFP) promoter, Arbuthnot et al., Hum. Gene Ther., 7:1503-14 (1996)), bone osteocalcin promoter (Stein et al., Mol. Biol. Rep., 24:185-96 (1997)); bone sialoprotein promoter (Chen et al., J. Bone Miner. Res., 11:654-64 (1996)), CD2 promoter (Hansal et al., J. Immunol., 161:1063-8 (1998); immunoglobulin heavy chain promoter; T cell receptor α-chain promoter, neuronal such as neuron-specific enolase (NSE) promoter (Andersen et al., Cell. Mol. Neurobiol., 13:503-15 (1993)), neurofilament light-chain gene promoter (Piccioli et al., Proc. Natl. Acad. Sci. USA, 88:5611-5 (1991)), and the neuron-specific vgf gene promoter (Piccioli et al., Neuron, 15:373-84 (1995)), among others which will be apparent to the skilled artisan.

In some embodiments, a tissue-specific promoter is a bone tissue-specific promoter. Examples of bone tissue-specific promoters include but are not limited to promoters of osterix, osteocalcin, type 1 collagen al, DMP1, cathepsin K, Rank, etc. In some embodiments, a promoter is an osteoblast-specific promoter. In some embodiments, an osteoblast-specific promoter comprises an osteocalcin (OCN) promoter. In some embodiments, an OCN promoter comprises the nucleic acid sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, or are 100% identical, including all values in between to SEQ ID NO: 4. In some embodiments, an OCN promoter comprises the nucleic acid sequence set forth in SEQ ID NO: 4. In some embodiments, a promoter is an osteoclast-specific promoter. In some embodiments, an osteoclast-specific promoter comprises a RANK promoter or NFκB promoter, such as a PB2 promoter. In some embodiments, a RANK promoter comprises the nucleic acid sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, or are 100% identical, including all values in between to SEQ ID NO: 5. In some embodiments, a RANK promoter comprises the nucleic acid sequence set forth in SEQ ID NO: 5.

Aspects of the disclosure relate to an isolated nucleic acid comprising more than one promoter (e.g., 2, 3, 4, 5, or more promoters). For example, in the context of a construct having a transgene comprising a first region encoding a protein and an second region encoding an inhibitory RNA (e.g., miRNA), it may be desirable to drive expression of the protein coding region using a first promoter sequence (e.g., a first promoter sequence operably linked to the protein coding region), and to drive expression of the inhibitory RNA encoding region with a second promoter sequence (e.g., a second promoter sequence operably linked to the inhibitory RNA encoding region). Generally, the first promoter sequence and the second promoter sequence can be the same promoter sequence or different promoter sequences. In some embodiments, the first promoter sequence (e.g., the promoter driving expression of the protein coding region) is a RNA polymerase III (polIII) promoter sequence. Non-limiting examples of polIII promoter sequences include U6 and H1 promoter sequences. In some embodiments, the second promoter sequence (e.g., the promoter sequence driving expression of the inhibitory RNA) is a RNA polymerase II (polII) promoter sequence. Non-limiting examples of polII promoter sequences include T7, T3, SP6, RSV, and cytomegalovirus promoter sequences. In some embodiments, a polIII promoter sequence drives expression of an inhibitory RNA (e.g., miRNA) encoding region. In some embodiments, a polII promoter sequence drives expression of a protein coding region.

Recombinant AAVs (rAAVs)

The isolated nucleic acids of the disclosure may be recombinant adeno-associated virus (AAV) vectors (rAAV vectors). In some embodiments, an isolated nucleic acid as described by the disclosure comprises a region (e.g., a first region) comprising a first adeno-associated virus (AAV) inverted terminal repeat (ITR), or a variant thereof. The isolated nucleic acid (e.g., the recombinant AAV vector) may be packaged into a capsid protein and administered to a subject and/or delivered to a selected target cell. “Recombinant AAV (rAAV) vectors” are typically composed of, at a minimum, a transgene and its regulatory sequences, and 5′ and 3′ AAV inverted terminal repeats (ITRs). The transgene may comprise, as disclosed elsewhere herein, one or more regions that encode one or more proteins and/or inhibitory nucleic acids (e.g., shRNA, miRNAs, etc.) comprising a nucleic acid that targets an endogenous mRNA of a subject. The transgene may also comprise a region encoding, for example, a protein and/or an expression control sequence (e.g., a poly-A tail), as described elsewhere in the disclosure.

Generally, ITR sequences are about 145 bp in length. Preferably, substantially the entire sequences encoding the ITRs are used in the molecule, although some degree of minor modification of these sequences is permissible. The ability to modify these ITR sequences is within the skill of the art. (See, e.g., texts such as Sambrook et al., “Molecular Cloning. A Laboratory Manual”, 2d ed., Cold Spring Harbor Laboratory, New York (1989); and K. Fisher et al., J Virol., 70:520 532 (1996)). An example of such a molecule employed in the present invention is a “cis-acting” plasmid containing the transgene, in which the selected transgene sequence and associated regulatory elements are flanked by the 5′ and 3′ AAV ITR sequences. The AAV ITR sequences may be obtained from any known AAV, including presently identified mammalian AAV types. In some embodiments, the isolated nucleic acid (e.g., the rAAV vector) comprises at least one ITR having a serotype selected from AAV1, AAV2, AAV3, AAV4, AAV5, AAV6, AAV6.2, AAV7, AAV8, AAVrh8, AAV9, AAVrh10, AAVrh39, AAVrh43, AAV2/2-66, AAV2/2-84, AAV2/2-125, and variants thereof. In some embodiments, the isolated nucleic acid comprises a region (e.g., a first region) encoding an AAV2 ITR.

In some embodiments, the isolated nucleic acid further comprises a region (e.g., a second region, a third region, a fourth region, etc.) comprising a second AAV ITR. In some embodiments, the second AAV ITR has a serotype selected from AAV1, AAV2, AAV3, AAV4, AAV5, AAV6, AAV6.2, AAV7, AAV8, AAVrh8, AAV9, AAVrh10, AAVrh39, AAVrh43, AAV2/2-66, AAV2/2-84, AAV2/2-125, and variants thereof. In some embodiments, the second ITR is a mutant ITR that lacks a functional terminal resolution site (TRS). The term “lacking a terminal resolution site” can refer to an AAV ITR that comprises a mutation (e.g., a sense mutation such as a non-synonymous mutation, or missense mutation) that abrogates the function of the terminal resolution site (TRS) of the ITR, or to a truncated AAV ITR that lacks a nucleic acid sequence encoding a functional TRS (e.g., a ATRS ITR). Without wishing to be bound by any particular theory, a rAAV vector comprising an ITR lacking a functional TRS produces a self-complementary rAAV vector, for example as described by McCarthy (2008) Molecular Therapy 16(10):1648-1656.

As used herein, the term “self-complementary AAV vector” (scAAV) refers to a vector containing a double-stranded vector genome generated by the absence of a terminal resolution site (TR) from one of the ITRs of the AAV. The absence of a TR prevents the initiation of replication at the vector terminus where the TR is not present. In general, scAAV vectors generate single-stranded, inverted repeat genomes, with a wild-type (wt) AAV TR at each end and a mutated TR (mTR) in the middle. The instant invention is based, in part, on the recognition that DNA fragments encoding RNA hairpin structures (e.g. shRNA, miRNA, and ami-RNA) can serve a function similar to a mutant inverted terminal repeat (mTR) during viral genome replication, generating self-complementary AAV vector genomes. For example, in some embodiments, the disclosure provides rAAV (e.g. self-complementary AAV; scAAV) vectors comprising a single-stranded self-complementary nucleic acid with inverted terminal repeats (ITRs) at each of two ends and a central portion comprising a promoter operably linked with a sequence encoding a hairpin-forming RNA (e.g., shRNA, miRNA, ami-RNA, etc.). In some embodiments, the sequence encoding a hairpin-forming RNA (e.g., shRNA, miRNA, ami-RNA, etc.) is substituted at a position of the self-complementary nucleic acid normally occupied by a mutant ITR.

“Recombinant AAV (rAAV) vectors” are typically composed of, at a minimum, a transgene and its regulatory sequences, and 5′ and 3′ AAV inverted terminal repeats (ITRs). It is this recombinant AAV vector which is packaged into a capsid protein and delivered to a selected target cell. In some embodiments, the transgene is a nucleic acid sequence, heterologous to the vector sequences, which encodes a polypeptide, protein, functional RNA molecule (e.g., miRNA, miRNA inhibitor) or other gene product, of interest. The nucleic acid coding sequence is operatively linked to regulatory components in a manner which permits transgene transcription, translation, and/or expression in a cell of a target tissue.

The instant disclosure provides a vector comprising a single, cis-acting wild-type ITR. In some embodiments, the ITR is a 5′ ITR. In some embodiments, the ITR is a 3′ ITR. Generally, ITR sequences are about 145 bp in length. Preferably, substantially the entire sequences encoding the ITR(s) is used in the molecule, although some degree of minor modification of these sequences is permissible. The ability to modify ITR sequences is within the skill of the art. (See, e.g., texts such as Sambrook et al, “Molecular Cloning. A Laboratory Manual”, 2d ed., Cold Spring Harbor Laboratory, New York (1989); and K. Fisher et al., J Virol., 70:520 532 (1996)). For example, an ITR may be mutated at its terminal resolution site (TR), which inhibits replication at the vector terminus where the TR has been mutated and results in the formation of a self-complementary AAV. Another example of such a molecule employed in the present disclosure is a “cis-acting” plasmid containing the transgene, in which the selected transgene sequence and associated regulatory elements are flanked by the 5′ AAV ITR sequence and a 3′ hairpin-forming RNA sequence. AAV ITR sequences may be obtained from any known AAV, including presently identified mammalian AAV types. In some embodiments, an ITR sequence is an AAV2, AAV3, AAV4, AAV5, AAV6, AAV7, AAV8, AAVrh8, AAV9, AAV10, and/or AAVrh10 ITR sequence.

In some embodiments, the rAAVs of the disclosure are pseudotyped rAAVs. For example, a pseudotyped AAV vector containing the ITRs of serotype X encapsidated with the proteins of Y will be designated as AAVX/Y (e.g. AAV2/1 has the ITRs of AAV2 and the capsid of AAV1). In some embodiments, pseudotyped rAAVs may be useful for combining the tissue-specific targeting capabilities of a capsid protein from one AAV serotype with the viral DNA from another AAV serotype, thereby allowing targeted delivery of a transgene to a target tissue.

Methods for obtaining recombinant AAVs having a desired capsid protein are well known in the art. (See, for example, US 2003/0138772), the contents of which are incorporated herein by reference in their entirety). Typically the methods involve culturing a host cell which contains a nucleic acid sequence encoding an AAV capsid protein; a functional rep gene; a recombinant AAV vector composed of, AAV inverted terminal repeats (ITRs) and a transgene; and sufficient helper functions to permit packaging of the recombinant AAV vector into the AAV capsid proteins. In some embodiments, capsid proteins are structural proteins encoded by the cap gene of an AAV. AAVs comprise three capsid proteins, virion proteins 1 to 3 (named VP1, VP2 and VP3), all of which are transcribed from a single cap gene via alternative splicing. In some embodiments, the molecular weights of VP1, VP2 and VP3 are respectively about 87 kDa, about 72 kDa and about 62 kDa. In some embodiments, upon translation, capsid proteins form a spherical 60-mer protein shell around the viral genome. In some embodiments, the functions of the capsid proteins are to protect the viral genome, deliver the genome and interact with the host. In some aspects, capsid proteins deliver the viral genome to a host in a tissue specific manner.

In some embodiments, an AAV capsid protein is of an AAV serotype selected from the group consisting of AAV1, AAV2, AAV3, AAV4, AAV5, AAV6, AAV6.2, AAV7, AAV8, AAVrh8, AAV9, AAVrh10, AAVrh39, AAVrh43, AAV2/2-66, AAV2/2-84, AAV2/2-125. In some embodiments, an AAV capsid protein is of a serotype derived from a non-human primate, for example scAAV.rh8, AAV.rh39, or AAV.rh43 serotype. In some embodiments, an AAV capsid protein is of an AAV9 serotype.

The disclosure is based, in part, on rAAVs comprising capsid proteins that have increased tropism for bone tissue. In some embodiments, the capsid proteins are grafted to a bone-targeting peptide. A heterologous bone-targeting peptide may target OCs (e.g., specifically, or preferentially targets OCs relative to OBs) or OBs (e.g., specifically, or preferentially targets OBs relative to OCs). In some embodiments, a bone-targeting peptide comprises an (AspSerSer) n (where n is an integer between 2 and 100) peptide, which may also be referred to as a DSS peptide. In some embodiments, AAV capsid proteins comprising DSS peptides are described in PCT Publication WO 2019/183605, the entire contents of which are incorporated herein by reference. Further examples of bone-targeting peptides include but are not limited to those described by Ouyang et al. (2009) Lett. Organic Chem 6(4):272-277. In some embodiments, an rAAV described herein comprises a DSS-AAV9 capsid protein. In some embodiments, a DSS-AAV9 capsid protein comprises between 2 and 10 DSS repeats. In some embodiments, a DSS-AAV9 capsid protein comprises 2, 3, 4, 5, 6, 7, 8, 9, or 10 DSS repeats.

The components to be cultured in the host cell to package a rAAV vector in an AAV capsid may be provided to the host cell in trans. Alternatively, any one or more of the required components (e.g., recombinant AAV vector, rep sequences, cap sequences, and/or helper functions) may be provided by a stable host cell which has been engineered to contain one or more of the required components using methods known to those of skill in the art. Most suitably, such a stable host cell will contain the required component(s) under the control of an inducible promoter. However, the required component(s) may be under the control of a constitutive promoter. Examples of suitable inducible and constitutive promoters are provided herein, in the discussion of regulatory elements suitable for use with the transgene. In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contain the rep and/or cap proteins under the control of inducible promoters. Still other stable host cells may be generated by one of skill in the art.

The recombinant AAV vector, rep sequences, cap sequences, and helper functions required for producing the rAAV of the disclosure may be delivered to the packaging host cell using any appropriate genetic element (vector). The selected genetic element may be delivered by any suitable method, including those described herein. The methods used to construct any embodiment of this disclosure are known to those with skill in nucleic acid manipulation and include genetic engineering, recombinant engineering, and synthetic techniques. See, e.g., Sambrook et al., Molecular Cloning: A Laboratory Manual, Cold Spring Harbor Press, Cold Spring Harbor, N.Y. Similarly, methods of generating rAAV virions are well known and the selection of a suitable method is not a limitation on the present disclosure. See, e.g., K. Fisher et al., J. Virol., 70:520-532 (1993) and U.S. Pat. No. 5,478,745.

In some embodiments, recombinant AAVs may be produced using the triple transfection method (described in detail in U.S. Pat. No. 6,001,650). Typically, the recombinant AAVs are produced by transfecting a host cell with an recombinant AAV vector (comprising a transgene) to be packaged into AAV particles, an AAV helper function vector, and an accessory function vector. An AAV helper function vector encodes the “AAV helper function” sequences (i.e., rep and cap), which function in trans for productive AAV replication and encapsidation. Preferably, the AAV helper function vector supports efficient AAV vector production without generating any detectable wild-type AAV virions (i.e., AAV virions containing functional rep and cap genes). Non-limiting examples of vectors suitable for use with the present disclosure include pHLP19, described in U.S. Pat. No. 6,001,650 and pRep6cap6 vector, described in U.S. Pat. No. 6,156,303, the entirety of both incorporated by reference herein. The accessory function vector encodes nucleotide sequences for non-AAV derived viral and/or cellular functions upon which AAV is dependent for replication (i.e., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus.

In some aspects, the disclosure provides transfected host cells. The term “transfection” is used to refer to the uptake of foreign DNA by a cell, and a cell has been “transfected” when exogenous DNA has been introduced inside the cell membrane. A number of transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Such techniques can be used to introduce one or more exogenous nucleic acids, such as a nucleotide integration vector and other nucleic acid molecules, into suitable host cells.

A “host cell” refers to any cell that harbors, or is capable of harboring, a substance of interest. Often a host cell is a mammalian cell. In some embodiments, a host cell is a bacterial cell, yeast cell, insect cell (Sf9), or a mammalian (e.g., human, rodent, non-human primate, etc.) cell. A host cell may be used as a recipient of an AAV helper construct, an AAV minigene plasmid, an accessory function vector, or other transfer DNA associated with the production of recombinant AAVs. The term includes the progeny of the original cell which has been transfected. Thus, a “host cell” as used herein may refer to a cell which has been transfected with an exogenous DNA sequence. It is understood that the progeny of a single parental cell may not necessarily be completely identical in morphology or in genomic or total DNA complement as the original parent, due to natural, accidental, or deliberate mutation.

As used herein, the term “cell line” refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants.

As used herein, the terms “recombinant cell” refers to a cell into which an exogenous DNA segment, such as DNA segment that leads to the transcription of a biologically-active polypeptide or production of a biologically active nucleic acid such as an RNA, has been introduced.

In some aspects, the present disclosure provides a recombinant AAV comprising a capsid protein and an isolated nucleic acid comprising a first region encoding an AAV ITR and a second region comprising a transgene, wherein the transgene encodes an artificial microRNA. The artificial microRNA may decrease the expression of a target gene in a cell (e.g. osteoblasts, osteoclasts, osteocytes, chondrocytes) or a subject. In some embodiments, the rAAV comprises an artificial microRNA that decreases the expression of SHN3 in a cell or a subject.

Expression of the target gene (e.g., SHN3, SOST, CTSK, RANK, RANKL, etc.) in a cell or subject may be decreased by between 50% and 99% (e.g., any integer between 50% and 99%, inclusive) using rAAVs of the present disclosure. Expression of the target gene (e.g., SHN3, SOST, CTSK, RANK, RANKL, etc.) in a cell or subject may be decreased by between 75% and 90% using rAAVs of the present disclosure. Expression of the target gene (e.g., SHN3, SOST, CTSK, RANK, RANKL, etc.) in a cell or subject may be decreased by between 80% and 99% using rAAVs of the present disclosure.

The foregoing methods for packaging recombinant vectors in desired AAV capsids to produce the rAAVs of the disclosure are not meant to be limiting and other suitable methods will be apparent to the skilled artisan.

Modes of Administration and Compositions

The rAAVs of the disclosure may be delivered to a subject in compositions according to any appropriate methods known in the art. For example, an rAAV, preferably suspended in a physiologically compatible carrier (e.g., in a composition), may be administered to a subject, e.g., host animal, such as a human, mouse, rat, cat, dog, sheep, rabbit, horse, cow, goat, pig, guinea pig, hamster, chicken, turkey, or a non-human primate (e.g., Macaque). In some embodiments a host animal does not include a human.

Delivery of the rAAVs to a mammalian subject may be by, for example, intramuscular injection or by administration into the bloodstream of the mammalian subject. Administration into the bloodstream may be by injection into a vein, an artery, or any other vascular conduit. In some embodiments, the rAAVs are administered into the bloodstream by way of isolated limb perfusion, a technique well known in the surgical arts, the method essentially enabling the artisan to isolate a limb from the systemic circulation prior to administration of the rAAV virions. A variant of the isolated limb perfusion technique, described in U.S. Pat. No. 6,177,403, can also be employed by the skilled artisan to administer the virions into the vasculature of an isolated limb to potentially enhance transduction into muscle cells or tissue. Moreover, in certain instances, it may be desirable to deliver the virions to the bone (e.g., bone tissue) of a subject. By “bone tissue” is meant all cells and tissue of the bone and/or joint (e.g., cartilage, axial and appendicular bone, etc.) of a vertebrate. Thus, the term includes, but is not limited to, osteoblasts, osteocytes, osteoclasts, chondrocytes, and the like. Recombinant AAVs may be delivered directly to the bone by injection into, e.g., directly into the bone, via intrasynovial injection, knee injection, femoral intramedullary injection, etc., with a needle, catheter or related device, using surgical techniques known in the art. In some embodiments, rAAV as described in the disclosure are administered by intravenous injection. In some embodiments, the rAAV are administered by intramuscular injection.

Aspects of the instant disclosure relate to compositions comprising a recombinant AAV comprising a capsid protein and a nucleic acid encoding a transgene, wherein the transgene comprises a nucleic acid sequence encoding one or more bone metabolism modulating agents. In some embodiments, the nucleic acid further comprises one or more AAV ITRs.

In some embodiments, the rAAV comprises an rAAV vector comprising the sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, or are 100% identical, including all values in between to any one of SEQ ID NOs: 1-40. In some embodiments, the rAAV comprises an rAAV vector comprising the sequence set forth in any one of SEQ ID NO: 1-40 (or the complementary sequence thereof), or a portion thereof. In some embodiments, a composition further comprises a pharmaceutically acceptable carrier.

In some embodiments, compositions comprise a recombinant AAV comprising a capsid protein and a nucleic acid comprising a first region encoding an AAV ITR and a second region comprising a transgene, wherein the transgene encodes an artificial microRNA that targets SHN3. In some embodiments, the recombinant AAV comprises a sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, or are 100% identical, including all values in between to SEQ ID NO: 33, 36, or 37. In some embodiments, the recombinant AAV comprises a sequence as set forth in SEQ ID NO: 33, 36, or 37. In some embodiments, the capsid protein further comprises a heterologous bone-targeting peptide, for example a DSS-AAV9 capsid protein.

In some embodiments, compositions comprise a recombinant AAV comprising a capsid protein and a nucleic acid comprising a first region encoding an AAV ITR and a second region comprising a transgene, wherein the transgene encodes an artificial microRNA that targets SOST. In some embodiments, the recombinant AAV comprises a sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, or are 100% identical, including all values in between to any one of SEQ ID NOs: 26-31. In some embodiments, the recombinant AAV comprises a sequence as set forth in any one of SEQ ID NOs: 26-31. In some embodiments, the capsid protein further comprises a heterologous bone-targeting peptide, for example a DSS-AAV9 capsid protein.

In some embodiments, compositions comprise a recombinant AAV comprising a capsid protein and a nucleic acid comprising a first region encoding an AAV ITR and a second region comprising a transgene, wherein the transgene encodes an artificial microRNA that targets CTSK. In some embodiments, the recombinant AAV comprises a sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, or are 100% identical, including all values in between any one of SEQ ID NOs: 20-25. In some embodiments, the recombinant AAV comprises a sequence as set forth in any one of SEQ ID NOs: 20-25. In some embodiments, the capsid protein further comprises a heterologous bone-targeting peptide, for example a DSS-AAV9 capsid protein.

In some embodiments, compositions comprise a recombinant AAV comprising a capsid protein and a nucleic acid comprising a first region encoding an AAV ITR and a second region comprising a transgene, wherein the transgene encodes an artificial microRNA that targets RANKL. In some embodiments, the recombinant AAV comprises a sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, or are 100% identical, including all values in between any one of SEQ ID NOs: 12-19. In some embodiments, the recombinant AAV comprises a sequence as set forth in any one of SEQ ID NOs: 12-19. In some embodiments, the capsid protein further comprises a heterologous bone-targeting peptide, for example a DSS-AAV9 capsid protein.

In some embodiments, compositions comprise a recombinant AAV comprising a capsid protein and a nucleic acid comprising a first region encoding an AAV ITR and a second region comprising a transgene, wherein the transgene encodes an artificial microRNA that targets RANK. In some embodiments, the capsid protein further comprises a heterologous bone-targeting peptide, for example a DSS-AAV9 capsid protein.

In some embodiments, compositions comprise a recombinant AAV comprising a capsid protein and a nucleic acid comprising a first region encoding an AAV ITR and a second region comprising a transgene, wherein the transgene encodes a sTNFR2 protein. In some embodiments, the recombinant AAV comprises a sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, or are 100% identical, including all values in between to SEQ ID NO: 6. In some embodiments, the recombinant AAV comprises a sequence as set forth in SEQ ID NO: 6. In some embodiments, the capsid protein further comprises a heterologous bone-targeting peptide, for example a DS S-AAV9 capsid protein.

In some embodiments, compositions comprise a recombinant AAV comprising a capsid protein and a nucleic acid comprising a first region encoding an AAV ITR and a second region comprising a transgene, wherein the transgene encodes a sIL1Rαprotein. In some embodiments, the recombinant AAV comprises a sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, or are 100% identical, including all values in between to SEQ ID NO: 36. In some embodiments, the recombinant AAV comprises a sequence as set forth in SEQ ID NO: 36. In some embodiments, the capsid protein further comprises a heterologous bone-targeting peptide, for example a DSS-AAV9 capsid protein.

Aspects of the disclosure provide a method of decreasing target gene (e.g., SHN3, SOST, CTSK, RANK, RANKL, etc.) expression in a cell. A cell may be a single cell or a population of cells (e.g., culture). A cell may be in vivo (e.g., in a subject) or in vitro (e.g., in culture). A subject may be a mammal, optionally a human, a mouse, a rat, a non-human primate, a pig, a dog, a cat, a chicken, or a cow.

Expression of the target gene in a cell or subject may be decreased by between 50% and 99% (e.g., any integer between 50% and 99%, inclusive) using isolated nucleic acids, rAAVs, or compositions of the present disclosure. Expression of the target gene in a cell or subject may be decreased by between 75% and 90% using isolated nucleic acids, rAAVs, or compositions of the present disclosure. Expression of the target gene in a cell or subject may be decreased by between 80% and 99% using isolated nucleic acids, rAAVs, or compositions of the present disclosure.

The compositions of the disclosure may comprise an rAAV alone, or in combination with one or more other viruses (e.g., a second rAAV encoding having one or more different transgenes). In some embodiments, a composition comprises 1, 2, 3, 4, 5, 6, 7, 8, 9, 10, or more different rAAVs each having one or more different transgenes.

Suitable carriers may be readily selected by one of skill in the art in view of the indication for which the rAAV is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present disclosure.

Optionally, the compositions of the disclosure may contain, in addition to the rAAV and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

The rAAVs are administered in sufficient amounts to transfect the cells of a desired tissue and to provide sufficient levels of gene transfer and expression without undue adverse effects. Conventional and pharmaceutically acceptable routes of administration include, but are not limited to, direct delivery to the selected organ (e.g., intraportal delivery to the liver), oral, inhalation (including intranasal and intratracheal delivery), intraocular, intravenous, intramuscular, subcutaneous, intradermal, intratumoral, and other parental routes of administration. Routes of administration may be combined, if desired.

The dose of rAAV virions required to achieve a particular “therapeutic effect,” e.g., the units of dose in genome copies/per kilogram of body weight (GC/kg), will vary based on several factors including, but not limited to: the route of rAAV virion administration, the level of gene or RNA expression required to achieve a therapeutic effect, the specific disease or disorder being treated, and the stability of the gene or RNA product. One of skill in the art can readily determine a rAAV virion dose range to treat a patient having a particular disease or disorder based on the aforementioned factors, as well as other factors that are well known in the art.

An “effective amount” of an rAAV is an amount sufficient to target infect an animal, target a desired tissue (e.g., bone tissue). The effective amount will depend primarily on factors such as the species, age, weight, health of the subject, and the tissue to be targeted, and may thus vary among animal and tissue. For example, an effective amount of the rAAV is generally in the range of from about 1 ml to about 100 ml of solution containing from about 10 9 to 10 16 genome copies. In some cases, a dosage between about 10 11 to 10 13 rAAV genome copies is appropriate. In certain embodiments, 10 12 or 10 13 rAAV genome copies is effective to target bone tissue. In some embodiments, the number of vector genomes administered to the subject can be any dose that is suitable for the treatments and methods disclosed herein.

In some embodiments, a dose of rAAV is administered to a subject no more than once per calendar day (e.g., a 24-hour period). In some embodiments, a dose of rAAV is administered to a subject no more than once per 2, 3, 4, 5, 6, or 7 calendar days. In some embodiments, a dose of rAAV is administered to a subject no more than once per calendar week (e.g., 7 calendar days). In some embodiments, a dose of rAAV is administered to a subject no more than bi-weekly (e.g., once in a two calendar week period). In some embodiments, a dose of rAAV is administered to a subject no more than once per calendar month (e.g., once in 30 calendar days). In some embodiments, a dose of rAAV is administered to a subject no more than once per six calendar months. In some embodiments, a dose of rAAV is administered to a subject no more than once per calendar year (e.g., 365 days or 366 days in a leap year).

In some embodiments, rAAV compositions are formulated to reduce aggregation of AAV particles in the composition, particularly where high rAAV concentrations are present (e.g., ˜10 13 GC/ml or more). Methods for reducing aggregation of rAAVs are well known in the art and, include, for example, addition of surfactants, pH adjustment, salt concentration adjustment, etc. (See, e.g., Wright F R, et al., Molecular Therapy (2005) 12, 171-178, the contents of which are incorporated herein by reference.) Formulation of pharmaceutically-acceptable excipients and carrier solutions is well-known to those of skill in the art, as is the development of suitable dosing and treatment regimens for using the particular compositions described herein in a variety of treatment regimens.

Typically, these formulations may contain at least about 0.1% of the active compound or more, although the percentage of the active ingredient(s) may, of course, be varied and may conveniently be between about 1 or 2% and about 70% or 80% or more of the weight or volume of the total formulation. Naturally, the amount of active compound in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In certain circumstances it will be desirable to deliver the rAAV-based therapeutic constructs in suitably formulated pharmaceutical compositions disclosed herein either subcutaneously, intraopancreatically, intranasally, parenterally, intravenously, intramuscularly, intrathecally, femoral intramedullary, or orally, intraperitoneally, or by inhalation. In some embodiments, the administration modalities as described in U.S. Pat. Nos. 5,543,158; 5,641,515 and 5,399,363 (each specifically incorporated herein by reference in its entirety) may be used to deliver rAAVs. In some embodiments, a preferred mode of administration is by portal vein injection.

The pharmaceutical forms suitable for injectable use include sterile aqueous solutions or dispersions and sterile powders for the extemporaneous preparation of sterile injectable solutions or dispersions. Dispersions may also be prepared in glycerol, liquid polyethylene glycols, and mixtures thereof and in oils. Under ordinary conditions of storage and use, these preparations contain a preservative to prevent the growth of microorganisms. In many cases the form is sterile and fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the host. The person responsible for administration will, in any event, determine the appropriate dose for the individual host.

Sterile injectable solutions are prepared by incorporating the active rAAV in the required amount in the appropriate solvent with various of the other ingredients enumerated herein, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The rAAV compositions disclosed herein may also be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host.

Delivery vehicles such as liposomes, nanocapsules, microparticles, microspheres, lipid particles, vesicles, and the like, may be used for the introduction of the compositions of the present disclosure into suitable host cells. In particular, the rAAV vector delivered transgenes may be formulated for delivery either encapsulated in a lipid particle, a liposome, a vesicle, a nanosphere, or a nanoparticle or the like.

Such formulations may be preferred for the introduction of pharmaceutically acceptable formulations of the nucleic acids or the rAAV constructs disclosed herein. The formation and use of liposomes is generally known to those of skill in the art. Recently, liposomes were developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868 and 5,795,587).

Liposomes have been used successfully with a number of cell types that are normally resistant to transfection by other procedures. In addition, liposomes are free of the DNA length constraints that are typical of viral-based delivery systems. Liposomes have been used effectively to introduce genes, drugs, radiotherapeutic agents, viruses, transcription factors and allosteric effectors into a variety of cultured cell lines and animals. In addition, several successful clinical trials examining the effectiveness of liposome-mediated drug delivery have been completed.

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 Å, containing an aqueous solution in the core.

Alternatively, nanocapsule formulations of the rAAV may be used. Nanocapsules can generally entrap substances in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use. In addition to the methods of delivery described above, the following techniques are also contemplated as alternative methods of delivering the rAAV compositions to a host. Sonophoresis (i.e., ultrasound) has been used and described in U.S. Pat. No. 5,656,016 as a device for enhancing the rate and efficacy of drug permeation into and through the circulatory system. Other drug delivery alternatives contemplated are intraosseous injection (U.S. Pat. No. microchip devices (U.S. Pat. No. 5,797,898), ophthalmic formulations (Bourlais et al., 1998), transdermal matrices (U.S. Pat. Nos. 5,770,219 and 5,783,208) and feedback-controlled delivery (U.S. Pat. No. 5,697,899).

Therapeutic Methods

Methods for delivering an effective amount of a transgene (e.g., an isolated nucleic acid or rAAV encoding one or more bone metabolism modulating agent) to a subject are provided by the disclosure. In some embodiments, the methods comprise the step of administering to a subject an effective amount of an isolated nucleic acid encoding an interfering RNA capable of inhibiting bone loss (e.g., bone loss due to inflammatory conditions or disease). In some embodiments, the methods comprise the step of administering to a subject an effective amount of an isolated nucleic acid encoding an interfering RNA capable of inhibiting bone resorption. Thus, in some embodiments, isolated nucleic acids, rAAVs, and compositions described herein are useful for treating a subject having or suspected of having a disease or disorder associated with dysregulated bone metabolism.

As used herein, a “disease or disorder associated with dysregulated bone metabolism” refers to a condition characterized by an imbalance between bone deposition and bone resorption resulting in either 1) abnormally increased bone deposition (e.g., formation) relative to a healthy individual (e.g., a subject not having a disease characterized by imbalance between bone deposition and bone resorption), or 2) abnormally decreased bone deposition (e.g., formation) relative to a healthy individual (e.g., a subject not having a disease characterized by an imbalance between bone deposition and bone resorption), or 3) abnormally increased bone resorption (e.g., breakdown) relative to a healthy individual (e.g., a subject not having a disease characterized by imbalance between bone deposition and bone resorption), or 4) abnormally decreased bone resorption (e.g., breakdown) relative to a healthy individual (e.g., a subject not having a disease characterized by imbalance between bone deposition and bone resorption).

A “disease associated with reduced bone density” refers to a condition characterized by increased bone porosity resulting from either 1) abnormally decreased bone deposition (e.g., formation) relative to a healthy individual (e.g., a subject not having a disease characterized by decreased bone density), or 2) abnormally increased bone resorption (e.g., breakdown) relative to a healthy individual (e.g., a subject not having a disease characterized by decreased bone density). A disease associated with increased bone porosity may arise from either 1) abnormally decreased OB and/or osteocyte differentiation, function, or activity relative to a healthy individual (e.g., a subject not having a disease characterized by decreased bone density) and/or 2) abnormally increased OC differentiation, function, or activity relative to a healthy individual (e.g., a subject not having a disease characterized by decreased bone density). “Porosity” generally refers to the volume of fraction of bone not occupied by bone tissue.

A “disease associated with increased bone density” refers to a condition characterized by decreased bone porosity resulting from either 1) abnormally increased bone deposition (e.g., formation) relative to a healthy individual (e.g., a subject not having a disease characterized by increased bone density), or 2) abnormally decreased bone resorption (e.g., breakdown) relative to a healthy individual (e.g., a subject not having a disease characterized by increased bone density). A disease associated with decreased bone porosity may arise from either 1) abnormally increased OB and/or osteocyte differentiation, function, or activity relative to a healthy individual (e.g., a subject not having a disease characterized by increased bone density) and/or 2) abnormally decreased OC differentiation, function, or activity relative to a healthy individual (e.g., a subject not having a disease characterized by increased bone density).

Aspects of the present disclosure provide methods of treating a disease or disorder associated with dysregulated bone metabolism. Dysregulated bone metabolism may be diseases associated with reduced bone density (e.g., osteoporosis, critical sized-bone defects, a mechanical disorder resulting from disuse or injury). Dysregulated bone metabolism may be diseases associated with increased bone density (e.g., osteopetrosis, pycnodysostosis, sclerosteosis, acromegaly, fluorosis, myelofibrosis, hepatitis C-associated osteosclerosis, heterotrophic ossification).

In some embodiments, a subject having a disease or disorder associated with dysregulated bone metabolism has one or more signs or symptoms of an inflammatory disease.

Examples of inflammatory diseases include but are not limited to rheumatoid arthritis (RA), psoriasis, ankylosing spondylitis, systemic lupus erythematosus, multiple sclerosis, inflammatory bowel diseases, periodontitis, and pemphigus vulgaris. In some embodiments, a subject having an inflammatory disease is characterized as having an increased level or amount of inflammatory cytokines (e.g., interleukin-1 (IL-1), IL-6, IL-12, IL-17a, IL-17f, IL-18, IL-22, IL-23, tumor necrosis factor alpha (TNF-α), interferon gamma (IFNγ), granulocyte-macrophage colony stimulating factor (GM-CSF), etc.) or other markers of inflammation, relative to a normal, healthy subject. In some embodiment, the subject having an inflammatory disease has the level or amount of inflammatory cytokines (e.g., interleukin-1 (IL-1), IL-6, IL-12, IL-17a, IL-17f, IL-18, IL-22, IL-23, tumor necrosis factor alpha (TNF-α), interferon gamma (IFNγ), granulocyte-macrophage colony stimulating factor (GM-CSF), etc.) or other markers of inflammation increased by at least 5%, 10%, 20%, 30%, 40%, 50%, 60%, 70%, 80%, 90%, 100%, or by at least 2-fold, at least 5-fold, at least 10-fold, at least 20-fold, at least 50-fold, at least 100-fold, or at least 1000-fold compared to a normal, healthy subject.

As used herein, a “normal, healthy subject” refers to a subject who does not have, is not suspected of, or is at risk of developing a disease or disorder. In some embodiments, the disease or disorder is an inflammatory disease. In some embodiments, the disease or disorder is associated with bone metabolism. In some embodiments, a normal, healthy subject can be a control described herein.

As used herein, the term “treating” refers to the application or administration of a composition, isolated nucleic acid, vector, or rAAV as described herein to a subject having an inflammatory condition (e.g., RA, psoriasis, ankylosing spondylitis, systemic lupus erythematosus, multiple sclerosis, inflammatory bowel diseases, periodontitis, pemphigus vulgaris, etc.), or a predisposition toward an inflammatory condition (e.g., RA, psoriasis, ankylosing spondylitis, systemic lupus erythematosus, multiple sclerosis, inflammatory bowel diseases, periodontitis, pemphigus vulgaris, etc.), with the purpose to cure, heal, alleviate, relieve, alter, remedy, ameliorate, improve, or affect the disorder, the symptom of the disease, or the predisposition toward the inflammatory condition.

Alleviating an inflammatory condition includes delaying the development or progression of the disease, or reducing disease severity. Alleviating the disease does not necessarily require curative results. As used therein, “delaying” the development of a disease (such as RA, psoriasis, ankylosing spondylitis, systemic lupus erythematosus, multiple sclerosis, inflammatory bowel diseases, periodontitis, pemphigus vulgaris, etc.) means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of inflammatory diseases includes initial onset and/or recurrence.

In some embodiments, methods of treating a disease or disorder associated with a dysregulated bone metabolism comprise administering to a subject in need thereof a recombinant AAV (rAAV) comprising a transgene. A rAAV may comprise a modification that promotes its targeting to bone cells (e.g., osteoclasts and osteoblasts). Non-limiting modifications of rAAVs that promote its targeting to bone cells include modification of capsid proteins with heterologous bone-targeting peptides, modification of rAAV vectors with bone-specific promoters, and use of AAV serotypes with increased targeting to bone relative to other tissues.

In some embodiments, the rAAV comprising the heterologous bone-targeting peptide comprises a transgene which upregulates or downregulates a target gene associated with dysregulation of bone metabolism. In some embodiments, the transgene upregulates the expression of a target gene that is decreased in a disorder associated with reduced bone density (e.g., osteoporosis, critical sized-bone defects, a mechanical disorder resulting from disuse or injury). In some embodiments, the transgene downregulates the expression of a target gene that is increased in a disorder associated with reduced bone density. In some embodiments, the transgene upregulates the expression of a target gene that is decreased in a disorder associated with reduced bone density (e.g., osteoporosis, critical sized-bone defects, a mechanical disorder resulting from disuse or injury). In some embodiments, the transgene downregulates the expression of a target gene that is increased in a disorder associated with reduced bone density.

Aspects of the disclosure provide methods for treating a disease or disorder associated with a disease of disorder characterized by dysregulation of bone metabolism comprising administering to a subject a rAAV comprising a capsid protein and an isolated nucleic acid encoding an inhibitory nucleic acid. The rAAV may comprise an inhibitory nucleic acid (e.g., siRNA, shRNA, miRNA, or ami-RNA). The inhibitory nucleic acid may decrease or increase expression of a target gene associated with a disease or disorder characterized by dysregulation of bone metabolism.

In some embodiments, the present disclosure provides a method of treating disease or disorder associated with reduced bone density. The method comprises administering to a subject in need thereof a rAAV or an isolated nucleic acid comprising a transgene that targets a gene associated with reduced bone density. In some embodiments, the rAAV or isolated nucleic acid comprises a transgene encoding an artificial microRNA that targets a gene associated with reduced bone density. In some embodiments, the target gene is SHN3, SOST, CTSK, RANK, or RANKL.

Aspects of the present disclosure provide methods of treating a disease or disorder associated with reduced bone density comprising administering an rAAV or an isolated nucleic acid. In some embodiments, the rAAV comprises an rAAV vector comprising the sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity, or are 100% identical, including all values in between to any one of SEQ ID NOs: 1-40. In some embodiments, the rAAV or isolated nucleic acid comprise a sequence as set forth in any one of SEQ ID NOs: 1-40, or the complement thereof.

As disclosed herein, “identity” of sequences refers to the measurement or calculation of the percent of identical matches between two or more sequences with gap alignments addressed by a mathematical model, algorithm, or computer program that is known to one of ordinary skill in the art. The percent identity of two sequences (e.g., nucleic acid or amino acid sequences) may, for example, be determined using Basic Local Alignment Search Tool (BLAST®) such as NBLAST® and XBLAST® programs (version 2.0). Alignment technique such as Clustal Omega may be used for multiple sequence alignments. Other algorithms or alignment methods may include but are not limited to the Smith-Waterman algorithm, the Needleman—Wunsch algorithm, or Fast Optimal Global Sequence Alignment Algorithm (FOGSAA).

Expression of the target gene (e.g., SHN3, SOST, CTSK, RANK, RANKL, etc.) in a cell or subject may be decreased by between 50% and 99% (e.g., any integer between 50% and 99%, inclusive) using methods of the present disclosure. Expression of the target gene (e.g., SHN3, SOST, CTSK, RANK, RANKL, etc.) in a cell or subject may be decreased by between 75% and 90% using methods of the present disclosure. Expression of SHN3 in a cell or subject may be decreased by between 80% and 99% using methods of the present disclosure.

In some embodiments, an “effective amount” or “amount effective of a substance in the context of a composition or dose for administration to a subject refers to an amount sufficient to produce one or more desired effects (e.g., to transduce bone cells or bone tissue). In some embodiments, an effective amount of an isolated nucleic acid is an amount sufficient to transfect (or infect in the context of rAAV-mediated delivery) a sufficient number of target cells of a target tissue of a subject. In some embodiments, a target tissue is bone tissue (e.g., bone and bone tissue cells, such as OBs, OCs, osteocytes, chondrocytes, etc.). In some embodiments, an effective amount of an isolated nucleic acid (e.g., which may be delivered via an rAAV) may be an amount sufficient to have a therapeutic benefit in a subject, e.g., to increase activity or function of OBs and/or osteocytes, to inhibit activity of OBs and/or osteocytes, to increase activity of function of OCs, to inhibit activity or function of OCs, etc. In some embodiments, an effective amount of an isolated nucleic acid disclosed herein may partially or fully rescue bone losses. In some embodiments, an effective amount of an isolated nucleic acid disclosed herein may partially or fully alleviate the effects of the genes that cause bone losses. An effective amount can also involve delaying the occurrence of an undesired response. The effective amount will depend on a variety of factors such as, for example, the species, age, weight, health of the subject, the severity of a condition, the tissue to be targeted, the specific route of administration and like factors, and may thus vary among subject and tissue as described elsewhere in the disclosure.

Exemplary embodiments of the invention will be described in more detail by the following examples. These embodiments are exemplary of the invention, which one skilled in the art will recognize is not limited to the exemplary embodiments.

### Examples

In order that the invention described herein may be more fully understood, the following examples are set forth. The examples described in this application are offered to illustrate the systems and methods provided herein and are not to be construed in any way as limiting their scope.

### Example 1: Mouse Models with SHN3 Deletions Exhibited Reversed Inflammation-Induced Bone Loss

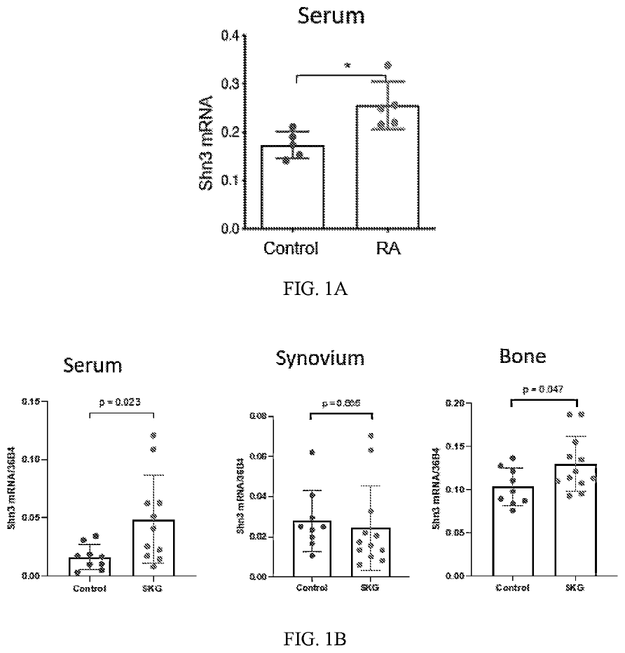

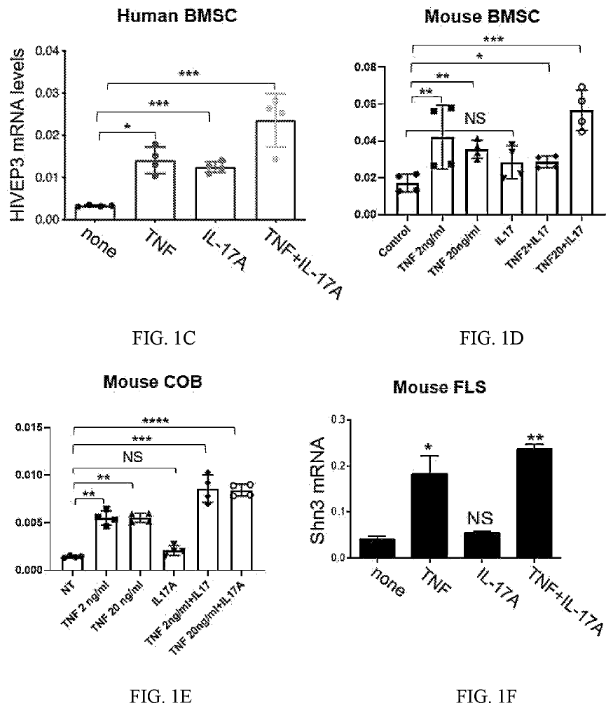

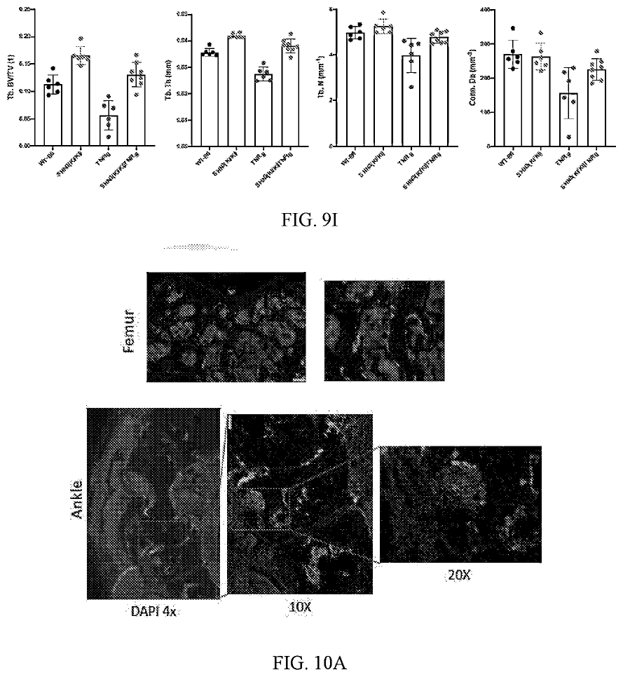

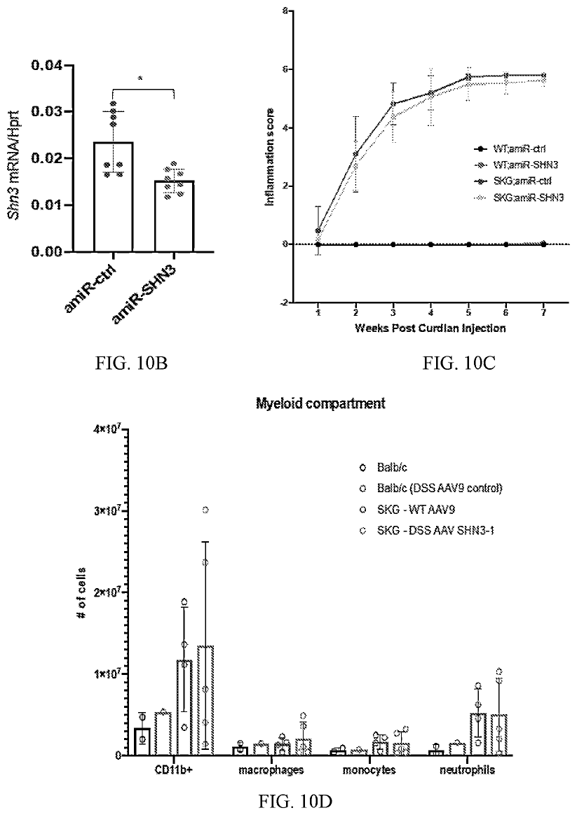

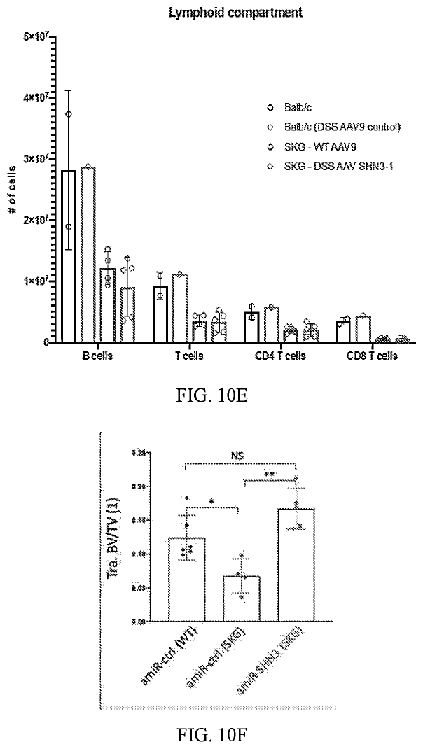

Rheumatoid arthritis (RA) is characterized by inflammation and destruction of articular and systemic bone. Osteoclasts (OCs), generated at the pannus-bone interface, are responsible for articular bone resorption in both human RA and in animal models. Intriguingly, in patients with RA, even when resorption is effectively arrested by therapeutic intervention, “repair” of erosions with deposition of new bone occurs in only about 10% of cases suggesting that the inflammatory arthritic environment also suppresses osteoblast (OB)-mediated bone formation, with uncoupling of bone resorption and formation. It has been observed that expression of Schnurri 3 (SHN3), a suppressor of WNT signaling in OBs, is elevated in the serum of RA patients relative to patients with psoriatic arthritis (PsA), another inflammatory arthritis in which these cytokines play a pathogenic role (FIG. 1A). The mouse model of inflammatory arthritis (SKG) displayed elevated levels of SHN3 mRNA in the serum and bone RNAs compared to wild type RNAs, while little to no difference was detected in the synovium RNA (FIG. 1B). In addition, human and mouse bone marrow-derived stromal cells (BMSCs) (FIG. 1C, 1D), mouse calvarial osteoblasts (COB, FIG. 1E), and mouse fibroblast-like synoviocytes (FLS) (FIG. 10 and FLS from RA patients are all responsive to treatment with TNF, IL-17A or their combination to induce SHN3 transcription. These results demonstrate that induction of SHN3 is accomplished by RA-associated cytokines, TNF and IL-17A.

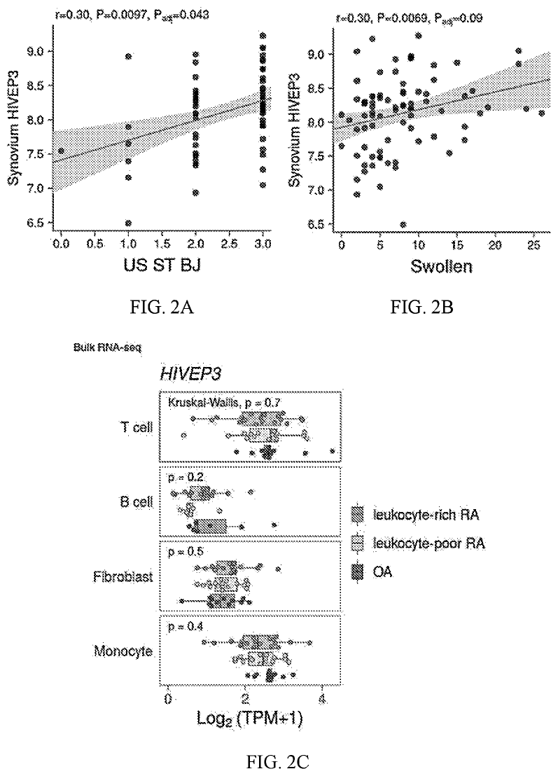

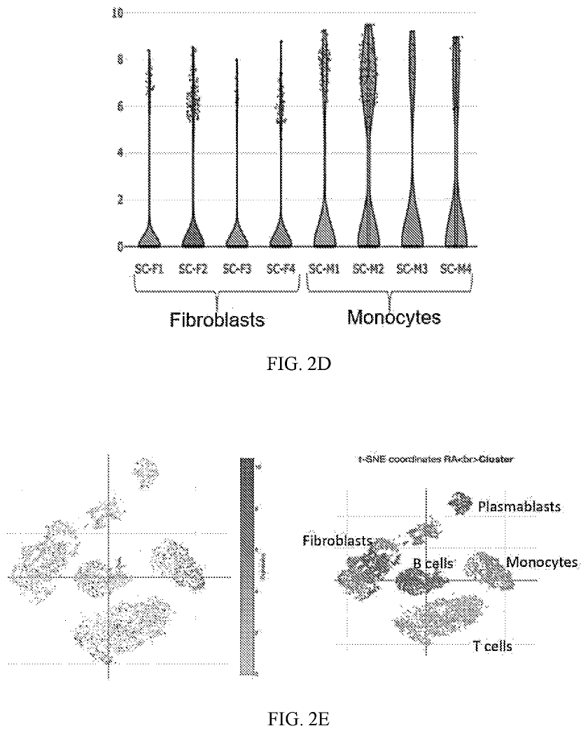

Transcriptome analysis of whole synovium from untreated early RA patients showed a correlation of SHN3 expression with synovitis scores on ultrasound (FIG. 2A) and with joint swelling (FIG. 2B). Moreover, T cells, B cells, FLS, and monocytes were isolated from the synovium of human patients with leukocyte-rich or -poor RA or from healthy synovium, demonstrating little to no difference in SHN3 mRNA levels (FIG. 2C). Using single cell transcriptome analysis, it was observed that FLS, monocytes, B cells, T cells, and plasmablasts from RA synovium express high levels of SHN3 mRNA (FIG. 2D, 2E). The mechanisms leading to impaired OB differentiation in RA and the role of SHN3 in RA pathogenesis remain to be fully elucidated.

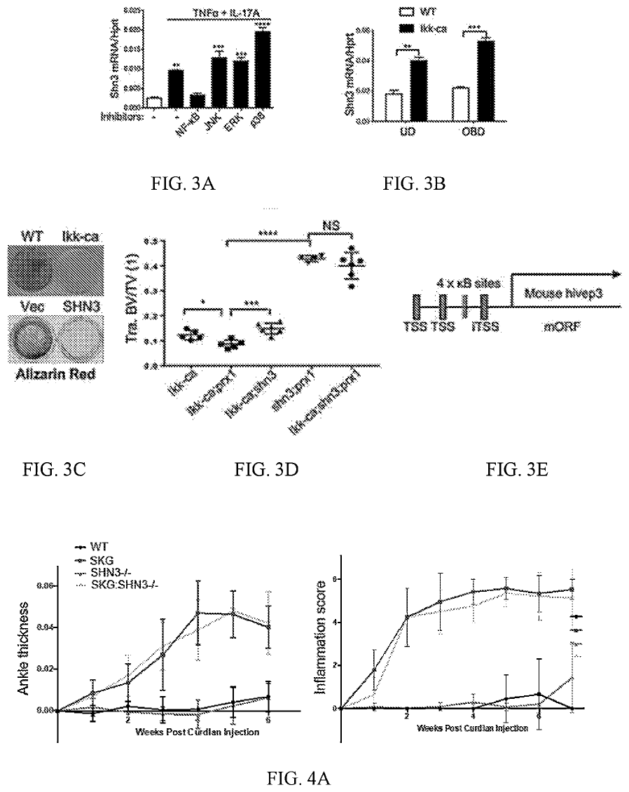

Human BMSCs show induction of SHN3 mRNA in response to TNF plus IL-17A, which was prevented by inhibition of the NF-κB pathway (FIG. 3A). SHN3 induction was relatively preserved in the presence of inhibitors of JNK, ERK, and p38 MAPKs, demonstrating that the NF-κB pathway, not MAPKs, mediates TNF/IL-17A-induced mRNA expression of SHN3 in OBs (FIG. 3A). Likewise, SHN3 expression was markedly upregulated in OBs expressing the constitutively active mutant of the IκB kinase β (Ikk-ca) compared to WT OBs (FIG. 3B). Since the NF-κB pathway functions as a negative regulator of OB differentiation (FIG. 3C), OB-specific activation of the NF-κB pathway by expressing IKK-CA (Ikk-ca,prx1) results in a significant reduction of femoral bone mass compared to WT controls (Ikk-ca^{fl/fl}, Ikk-ca^{fl/fl}; shn3^{fl/fl}). This bone loss was reversed by OB-specific deletion of SHN3 (Ikk-ca; shn3,prx1) (FIG. 3D). Of note, multiple KB-binding sites for the heterocomplex of RelA (p65) and NF-κB1 (p50) are located at 10, 120, 360, and 390 bp away from the 2nd transcription start site (TSS) in the promoter region of the mouse shn3 gene (FIG. 3E). Thus, data indicates activation of the NF-κB (p65/p50) heterocomplex by TNF plus IL-17A upregulates transcription of SHN3 in OBs via p65/p50 binding to the shn3 promoter. In turn, overexpressed SHN3 suppresses OB differentiation (FIG. 3C, bottom).

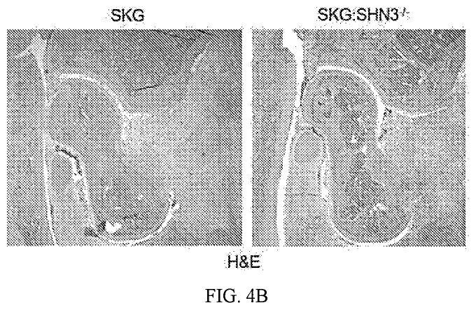

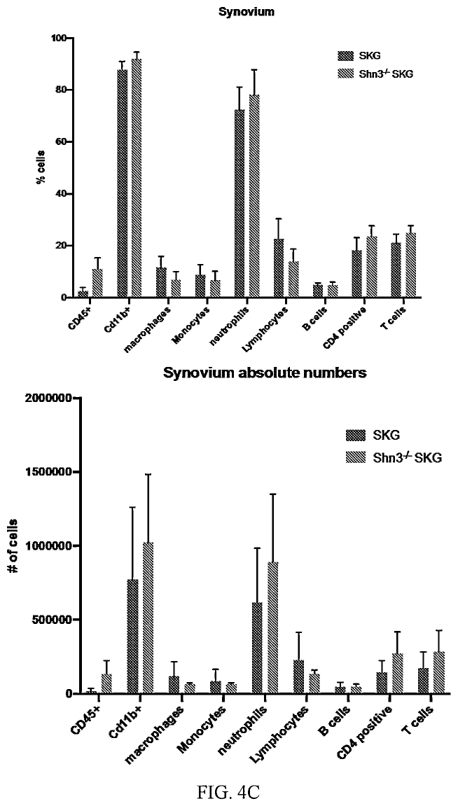

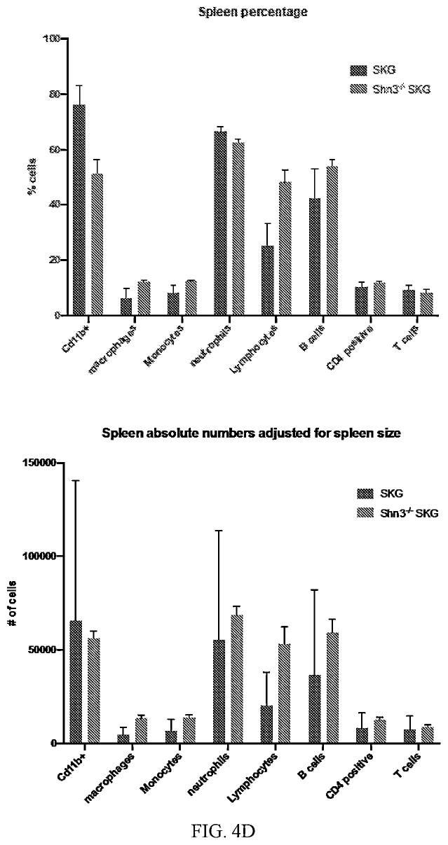

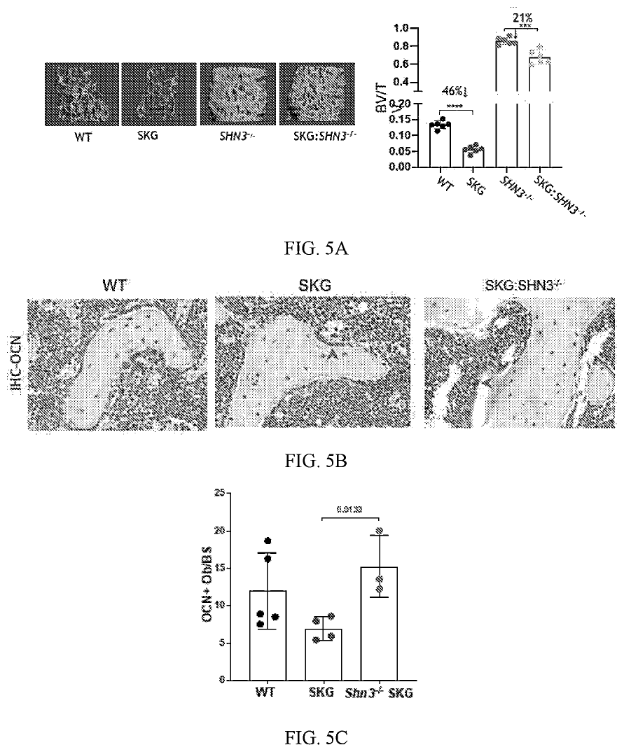

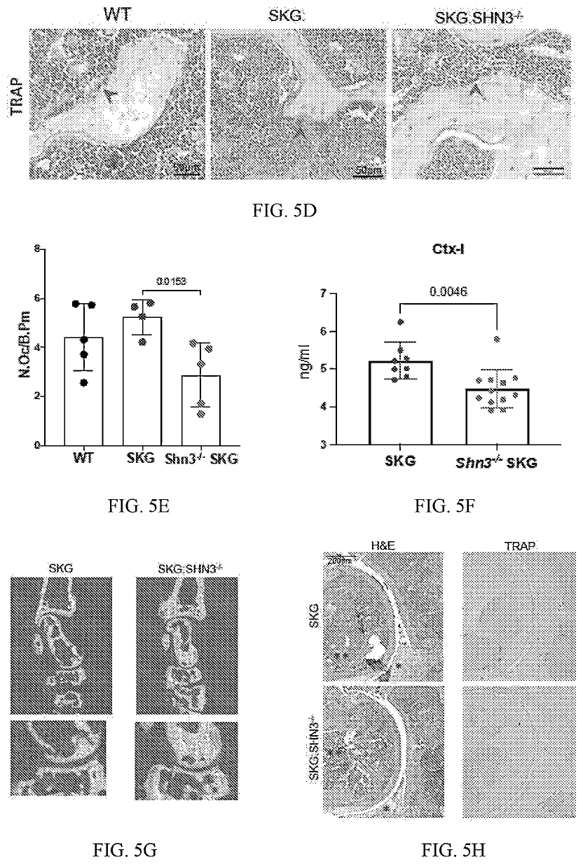

An association between SHN3 expression and severity of inflammation in RA (FIGS. 2A-2C) was observed. SHN3 is also expressed in RA synovium and serum and TNF plus IL-17A upregulate expression of SHN3 in OBs and FLS (FIG. 1). To test in vivo role of SHN3 in inflammatory arthritis in which both TNF and IL-17A play a pathogenic role, mice with germline deletion of SHN3 (shn3^{-/-}) were crossed with SKG mice in which a mutation in the ZAP-70 protein attenuates signaling through the T cell receptor and increases autoreactivity of peripheral T cells. Onset of disease in these mice can be timed with systemic injection of 1,3-beta-glucan (curdlan), causing the reproducible development of inflammation in the paw joints, induction of TNF, IL-17, IL-1 and IL-6 in synovium, and resulting in OC-mediated joint destruction and reduced systemic bone density. While little to no joint inflammation was detected in curdlan-treated BALB/c control (black) and shn3^{-/-} mice, curdlan accelerates joint and systemic inflammation in SKG and shn3^{-/-}: SKG mice (FIG. 4A, 4B). Of note, a flow cytometer analysis of synovial cells and spleenotyes demonstrated that immune cells, including macrophages, monocytes, neutrophils, lymphocytes, B cells, and T cells, were comparable between SKG and shn3^{-/-}; SKG mice (FIG. 4C, D), suggesting that SHN3 is dispensable of both local inflammation in the synovium and systemic inflammation. In contrast to inflamed SKG mice, which suffer a 46% reduction of trabecular bone mass in the femur, only a 21% reduction is observed in SKG mice in the absence of SHN3 (FIG. 5A). Accordingly, while SKG mice showed decreased numbers of active osteocalcin-expressing OBs (OCN±OB) and increased numbers of TRAP+OCs on the surface of femoral bone, numbers of OCN±OBs and TRAP+OCs were reversed by SHN3-deletion (FIG. 5B-5E). This is consistent with reduced serum levels of C-terminal telopeptide of type I collagen (Ctx-I) in shn3^{-/-}; SKG mice relative to SKG mice (FIG. 5F). These results suggest that SHN3 deletion can protect bone-residing OBs from inflammation and suppress inflammation-induced activation of OCs simultaneously, thereby preventing inflammation-induced bone loss. In addition to systemic bone loss (i.e., osteoporosis), SHN3 deletion protects articular bone erosion in the inflamed ankles of SKG mice. MicroCT analysis revealed that articular bone erosion was almost completely protected in shn3^{-/-}; SKG ankles relative to SKG ankles showing multiple erosion pits (FIG. 5G). This is accompanied with histologic analyses showing significant protection from articular bone erosion within inflamed ankles of shn3^{-/-}; SKG mice relative to SKG mice, while joint inflammation is comparable (FIG. 5H, 5I). Notably, numbers of TRAP+OCs were markedly reduced in inflamed ankles of shn3^{-/-}; SKG mice relative to SKG mice (FIG. 5H), demonstrating suppression of OC activation by SHN3 deletion. These results indicate that SHN3-deletion is effective to prevent both systemic bone loss and focal articular bone erosion in the setting of inflammatory arthritis without any alteration in inflammation, at least in part by an augmentation of OB activity and bone formation in shn3^{-/-}; SKG mice.

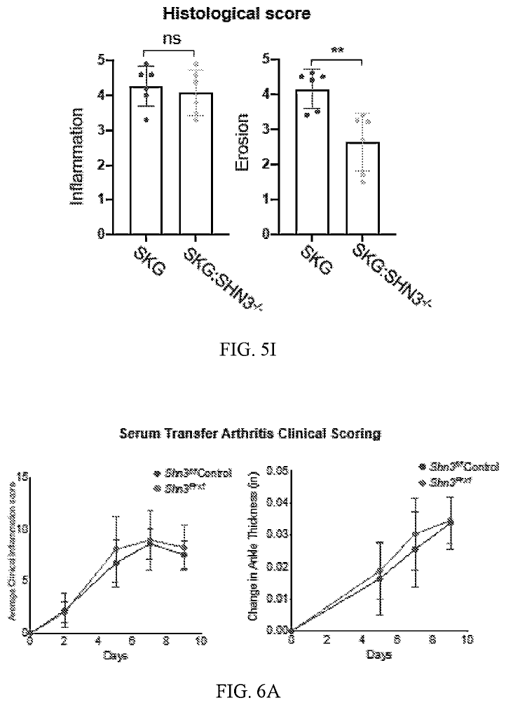

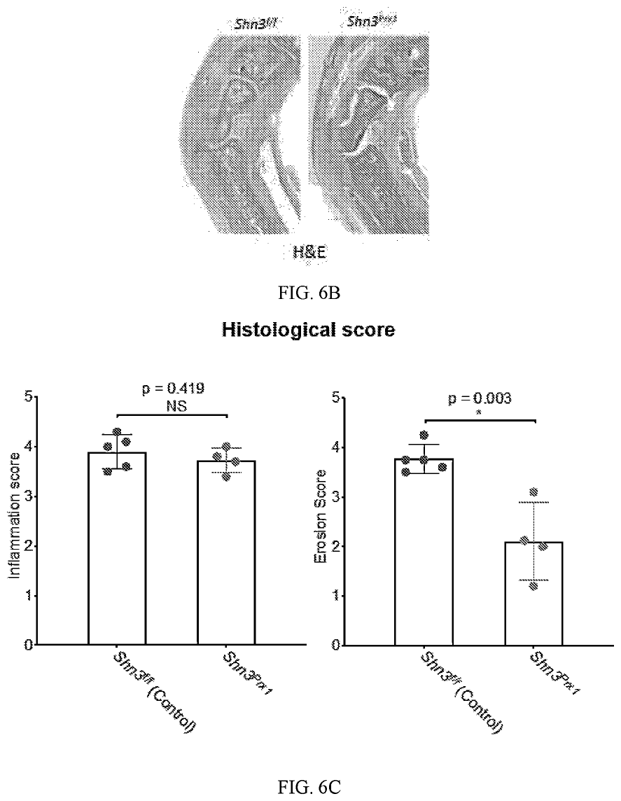

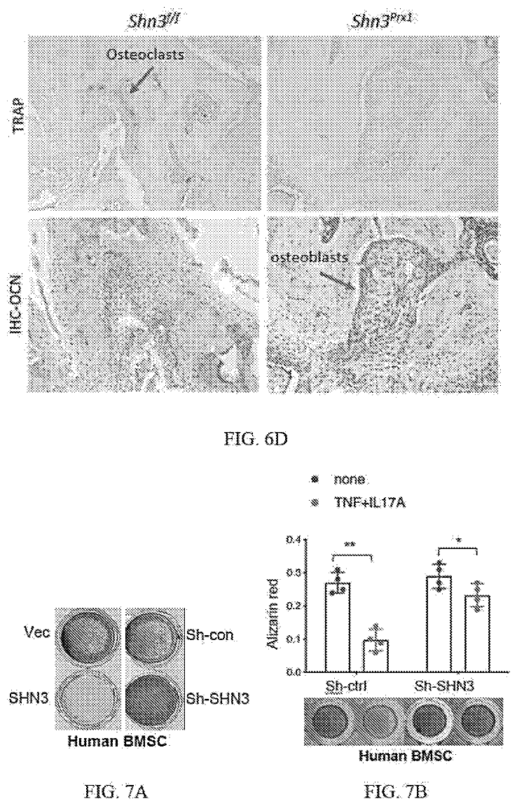

The K/BxN serum transfer arthritis (STA) model is a derivative of the KRN spontaneous arthritis model. The K/BxN mouse develops spontaneous autoimmune arthritis that mimics human RA, with leukocyte invasion in joints, pannus formation, cartilage destruction and bone erosion. This model was modified by injecting only two doses of arthritogenic serum and following the mice over time. In this modification, inflammation peaks 10 days post initial injection and resolves completely by 28-30 days. To test whether SHN3-deficient OBs can prevent articular bone erosions at peak inflammation, 5-week-old male SHN3 (f/f) and SHN3 (f/f);prx1 mice (n=5 mice/group) were i.p. injected with K/BxN serum, and inflammation and osteolysis in the ankle joint were assessed 10 days post-injection. Injection of K/BxN serum leads to development of joint inflammation in both SHN3 (f/f) and SHN3 (f/f);prx1 mice (FIG. 6A), demonstrating that joint inflammation is not affected by SHN3-deletion.. Of note, histologic analysis demonstrated no difference in infiltration of immune cells to inflamed ankle joints (FIGS. 6B and 6C, left) while a significant protection from joint erosion, along with an increase in OB-mediated periosteal bone formation and a decrease in OC-mediated bone resorption at erosion sites was observed in SHN3 (f/f);prx1 mice (FIG. 6C, right). In the pannus of SHN3 (f/f) mice, numbers of TRAP+OCs are markedly increased along with reduced numbers of OCN+OBs. By contrast, SHN3 (f/f);prx1 mice show decreased numbers of TRAP+OCs and increased numbers of OCN+OBs in the areas of pannus (FIG. 6D). These data indicate that SHN3-deficient OBs in the pannus might be resistant from RA cytokine-induced suppression and can interfere with the differentiation and/or recruitment of OCs to the pannus.

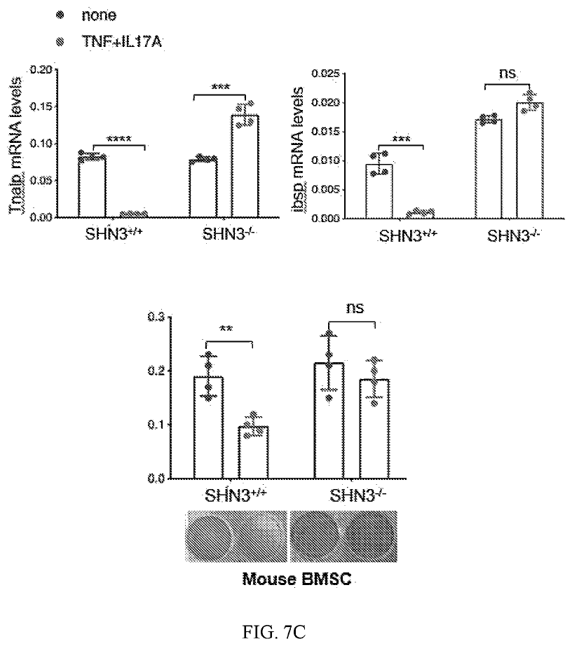

Because SHN3 expression was upregulated in OBs when treated with OB-suppressing cytokines, TNF and IL-17A, and overexpression of SHN3 similarly ablated OB differentiation in human BMSCs, whereas OB differentiation was markedly increased in SHN3-deficient MSCs (FIG. 7A), it was hypothesized that SHN3 mediates TNF+IL-17A-induced suppression of OB differentiation. While mineralization and osteogenic gene expression were both markedly reduced in human BMSCs (FIG. 7B) and mouse calvarial OBs (COBs) (FIG. 7C), SHN3-deficiency protected from suppression of OB differentiation by TNF+IL-17A treatment. These results may explain the mechanisms by which SHN3-deficiency may protect systemic bone loss and articular bone erosion that results from elevated levels of RA-associated cytokines in the setting of inflammatory arthritis (SKG mouse model).

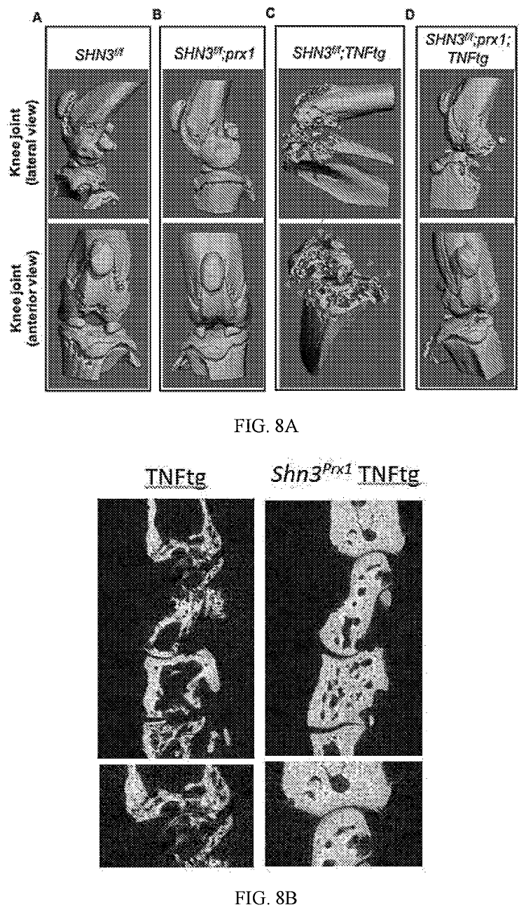

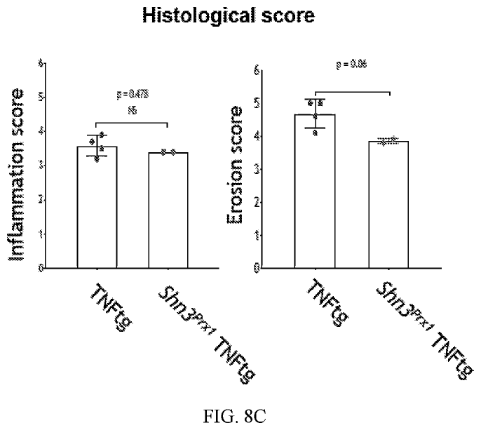

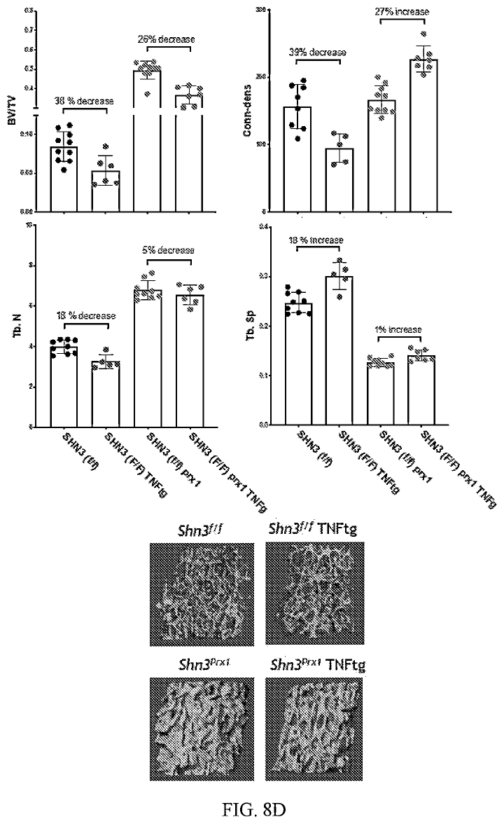

To test this hypothesis in vivo, SHN3 was conditionally deleted in OB lineage cells that overexpress human TNF by crossing SHN3 (f/f);prx1 mice with mice with overexpression of human TNF (TNF-tg). TNF-tg mice displayed an erosive polyarthritis and systemic osteoporosis mimicking that observed in RA. These mice developed inflammatory arthritis by 3-4 weeks of age in ankles, paws and knees that resulted in severe articular bone erosion, often debilitating by weeks of age. These mice began to develop systemic bone loss by 4 weeks of age (FIG. 8A, 8B). In contrast to TNFtg mice, microCT analysis of SHN3 (f/f);prx1; TNFtg mice revealed that articular bone erosion at knee joints (FIG. 8A) and ankles (FIGS. 8B and 8C, right) was markedly reduced in the absence of SHN3 while joint inflammation is comparable between TNFtg and SHN3 (f/f);prx1; TNFtg mice (FIG. 8C, left). Additionally, unlike TNFtg mice, which suffered a significant reduction of trabecular bone mass in the femur, a mild reduction was observed in TNFtg mice in the absence of SHN3 (FIG. 8D). These results demonstrated that OB-specific deletion of SHN3 can prevent systemic bone loss and articular bone erosion in the background of TNF-tg mice, without any alteration in joint inflammation. OB-specific deletion of SHN3 was effective to prevent TNF-induced articular bone erosion in the joints. FIG. 8D (microCT analysis) show unlike TNFtg mice, which suffered a significant reduction of femoral bone mass, including trabecular bone volume per tissue volume (BV/TV), connective density (Conn-dens), number (Tb. N), and thickness (Tb. Th), only a mild reduction was observed in SHN3 (f/f);prx1; TNFtg mice, demonstrating that OB-specific deletion of SHN3 was effective to prevent systemic bone loss by TNF.

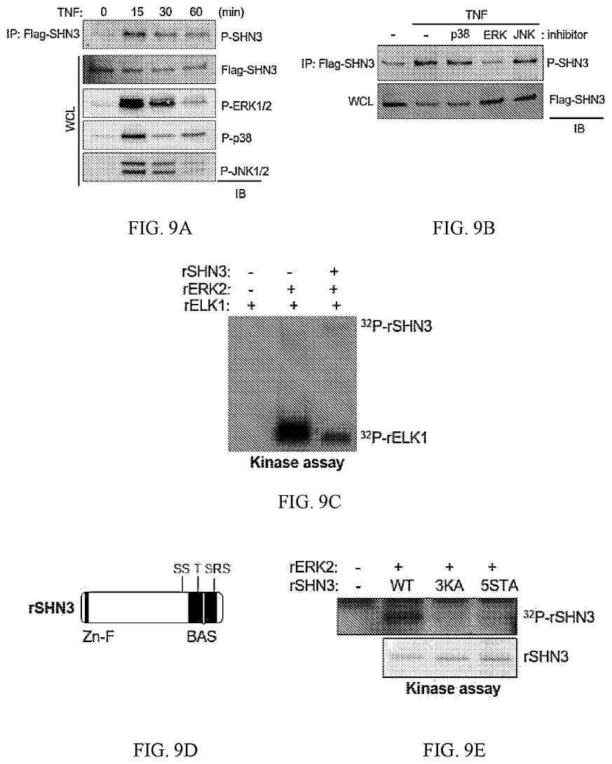

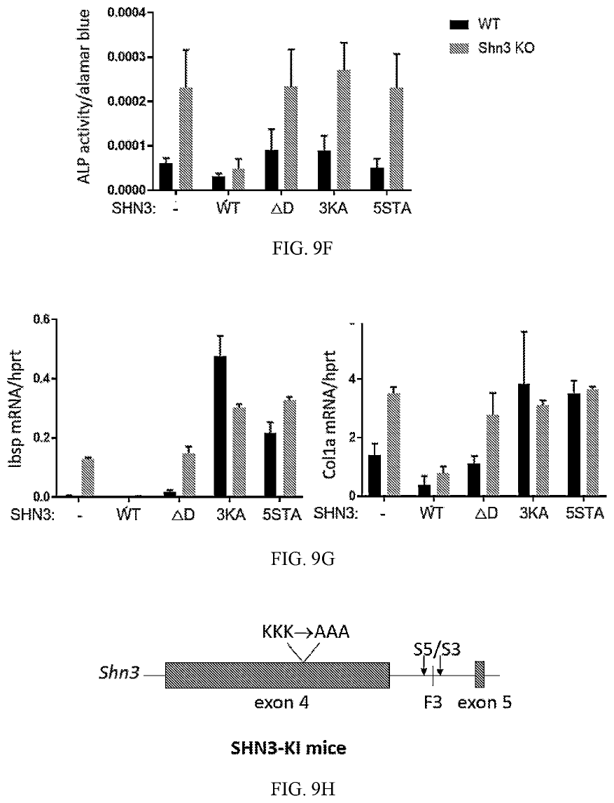

Since SHN3 mediates TNF and IL-17A-induced suppression of OB differentiation, post-translational regulation of SHN3 by these cytokines was investigated. Immunoblotting analysis revealed that phosphorylation of SHN3 peaks at 15 min post stimulation of TNF in OBs (FIG. 9A) and that its phosphorylation is markedly reduced when treated with an ERK inhibitor, but not inhibitors of p38 or JNK (FIG. 9B). However, SHN3 phosphorylation was not affected by IL-17A stimulation.

These results indicate that ERK MAPK activation by TNF, not IL-17A is required for phosphorylation of SHN3 in OBs. A MAPK-binding domain (the D-domain; PPKKKRARA, 884-892 aa) in the BAS domain of mouse SHN3 protein has previously been identified, and substitution of three lysines to alanines (SHN3-3KA) significantly reduced the ability of ERK MAPK to bind SHN3. Surprisingly, recombinant SHN3 (rSHN3) was phosphorylated by recombinant ERK2 (rERK2) while rSHN3 inhibited rERK2-induced phosphorylation of the ERK substrate ELK1 (FIG. 9C). Accordingly, phospho-spectrometry analysis of recombinant ERK MAPK (rERK) and recombinant SHN3 (rSHN3, 50-930 aa) revealed that rERK phosphorylates rSHN3 at two serines (810 and 811 aa), one threonine (851 aa), and two serines (911 and 913 aa) (FIG. 9D). When five phosphorylation sites (SHN3-5STA) or ERK-binding sites (SHN3-3KA) were all substituted to alanines, ERK-induced phosphorylation of SHN3 was markedly reduced (FIG. 9E), demonstrating that ERK phosphorylates SHN3 through this interaction. Importantly, alkaline phosphatase (ALP) activity (FIG. 9F) and osteogenic gene expression (FIG. 9G) were markedly decreased in both SHN3-sufficient and -deficient BMSCs by overexpression of SHN3-WT, but not SHN3 mutants that failed to bind ERK (SHN3-AD, SHN3-3KA) or be phosphorylated by ERK (SHN3-5STA), demonstrating that ERK-mediated phosphorylation is required for SHN3's function to inhibit OB differentiation. To test this observation in vivo, mice bearing a knock-in allele of SHN3-3KA in the endogenous shn3 locus (shn3^{Kl/Kl}) were generated (FIG. 9H) and crossed with TNF-tg mice, demonstrating that SHN3-3KA mutation protected from TNF-induced bone loss in the femur (FIG. 91). These results suggest that ERK-mediated phosphorylation of SHN3 is required for TNF-induced bone loss. RNAi-based bone anabolic gene therapy using recombinant adeno-associated virus (rAAV) was investigated. A modification of rAAV serotype 9 capsid that allows for homing of rAAV9 to bone, while de-targeting transduction to non-relevant tissues was developed and is referred to as DSS.rAAV9. This modified rAAV9 can deliver an artificial microRNA (ami-RNA, amiR) (amiR-SHN3) that silences shn3 within OBs residing in the bone, augments OB activity and promotes bone formation (e.g., DSS.rAAV9.amiR-SHN3). Additionally, it has been observed that systemic delivery of DSS.rAAV9.amiR-SHN3 in a mouse model of postmenopausal osteoporosis counteracted bone loss and enhanced bone mechanical properties. Likewise, DSS.rAAV9 is also effective for transduction of OCs in the cell culture and mice, and can deliver to OCs, an amiR that silences expression of a key OC regulator, cathepsin K (e.g., amiR-CTSK), to suppress OC-mediated bone resorption. It was observed that systemic delivery of DSS.rAAV9.amiR-CTSK reverses bone loss and improves bone mechanical properties in mouse models of postmenopausal and senile osteoporosis. Interestingly, systemically administered DSS.rAAV9.amiR-CTSK can simultaneously suppresses OC-mediated bone-resorption and promotes OB-mediated bone formation.

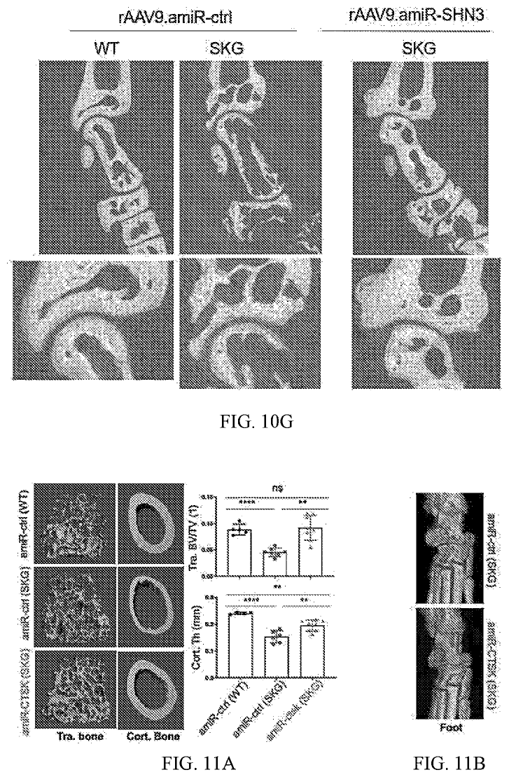

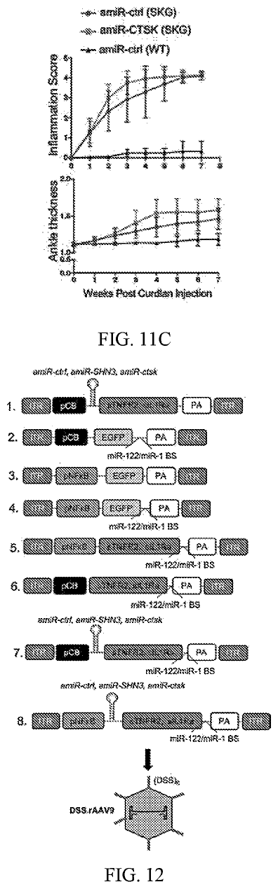

DSS.rAAV9.egfp-treated SKG mice were treated with curdlan to induce inflammatory arthritis. Two weeks later, AAV's tissue distribution was assessed by fluorescence microscopy (FIG. 10A), indicating AAV's transduction to OBs and OCs that reside in the femurs and the inflamed ankles. A single i.v. injection of DSS.rAAV9.amiR-SHN3 resulted in ˜40% reduction of shn3 mRNA levels in the tibial bone (FIG. 10B). Clinical inflammation scores and myeloid and lymphoid cell populations in the spleen were comparable between arthritic SKG mice expressing control and amiR-SHN3 (FIG. 10C-10E), demonstrating that local and systemic inflammation were not affected by AAV-mediated silencing of SHN3. Remarkably, while curdlan-induced inflammation induced a significant decrease in trabecular bone mass and cortical thickness in the femur of control-expressing SKG mice, AAV-mediated silencing of SHN3 in arthritic SKG mice prevented systemic bone loss (FIG. 10F) and articular bone erosion (FIG. 10G). These results suggest that SHN3 silencing by systemically delivered AAV9 is effective to protect both systemic osteoporosis and focal articular bone erosion in a mouse model of inflammatory arthritis. Similarly, systemic bone loss in the femur and peri-articular bone erosion in the ankle and foot of arthritic SKG mice were preserved when DSS.rAAV9.amiR-CTSK was i.v. injected (FIGS. 11A and B). However, joint inflammation was slightly increased by the treatment with DSS.rAAV9.amiR-CTSK (FIG. 11C). These results indicate that protection from inflammatory arthritis-induced bone loss via AAV-mediated silencing of SHN3 or CTSK occurs even in the presence of comparable levels of inflammation.

Since systemic and joint inflammation in arthritic SKG mice were not mitigated by AAV9-mediated silencing of SHN3 or CTSK, a polyvalent bone-targeting rAAV9 that can simultaneously prevent bone loss/erosion and suppress inflammation in inflammatory arthritis was produced. It has been previously reported that sTNFR2 and sIL1Rαcompetitively bind TNFα and IL-la/3 against their cognate receptors and therefore, suppress TNFα- and IL-1αβ-induced signal transduction, respectively. Thus, a secreted TNF antagonist, soluble human TNFR2 or a secreted IL-1 antagonist, soluble human IL1Ra was cloned into the AAV vector genome carrying amiR-ctrl, amiR-SHN3 or amiR-CTSK and packaged into DSS.rAAV9 capsid (FIG. 12; construct 1 includes: DSS.rAAV9.amiR-ctrl.sTNFR2 or sIL1Rα; DSS.rAAV9.amiR-SHN3.sTNFR2 or IL1Ra; DSS.rAAV9.amiR-CTSK.sTNFR2 or sIL1Rα). However, following systemic delivery, it was observed these vectors also target liver, heart, and skeletal muscle, in addition to bone. To circumvent this, tissue-specific, endogenous miRNAs to repress rAAV expression in the skeletal/heart muscle and liver, by engineering perfectly complementary miR-1 and miR-122-binding sites into the rAAV9 genome, respectively. Silencing of transgene expression in liver, heart, and muscle exploited the natural expression of the abundant (≥60,000 copies/cell) miRNAs, miR-122, which is expressed in hepatocytes, and miR-1, a miRNA found in the heart and skeletal muscle of virtually all animals.

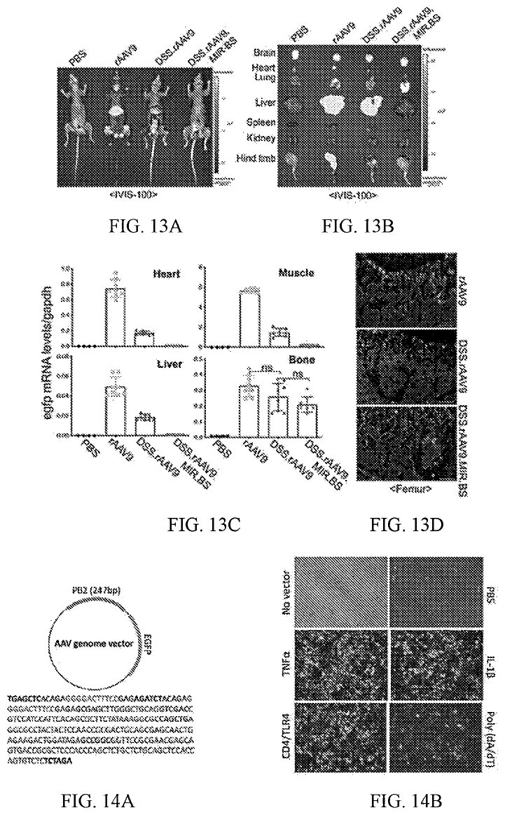

Bone-targeting rAAV9 that contains complementary miR-1 and miR-122-binding sites in the 3′-UTR of EGFP transgene (FIG. 12; e.g., Construct 2=DSS.rAAV9.MIR.BS) were produced. 2 weeks after a single i.v. injection of rAAV9, DSS.rAAV9 or DSS.rAAV9.MRI.BS vectors carrying EGFP transgene, their bio-distribution in whole body (FIG. 13A) and individual tissues (FIG. 13B) was assessed by EGFP expression using optical imaging system. Compared to rAAV9-treated mice showing high EGFP expression in heart, liver, and skeletal muscle, a modest expression in DSS.rAAV9-treated mice and little to no expression in DSS.rAAV9.MIR.BS were detected. qPCR analysis also shows a similar pattern of EGFP mRNA levels in these tissues (FIG. 13C). Notably, EGFP expression in the femur was relatively comparable between the treated mice (FIGS. 13C and D).

Since constitutive expression of anti-inflammatory proteins, including sTNFR2 and sIL1Rαcan make host cells susceptible to pathogenic infection, it is critical to limit their expression only when inflammatory arthritis occurs. Central to the pathogenesis of inflammatory arthritis is the activation of macrophages by autoreactive T cells, resulting in the release of pro-inflammatory cytokines, such as TNF, IL-1, IL-6, and IL-17. Biologic antibodies and/or small molecular compounds that target these cytokines, their cognate receptors or downstream signaling components have shown great efficacy in RA patients. Given that NF-κB activation is a key to initiate signal transduction downstream of these proinflammatory cytokines, the PB2 promoter containing two NF-κB-binding sites and one minimal FosP site was utilized to express EGFP in response to pro-inflammatory cytokines (FIG. 12; Construct 3 and FIG. 14A; PB2-GFP). EGFP expression was markedly upregulated in PB2-GFP vector-expressing HEK293 cells upon stimulation with various pro-inflammatory cytokines that activate the NF-κB pathway (FIG. 14B), indicating that PB2 promoter can drive a transgene expression in response to inflammation.

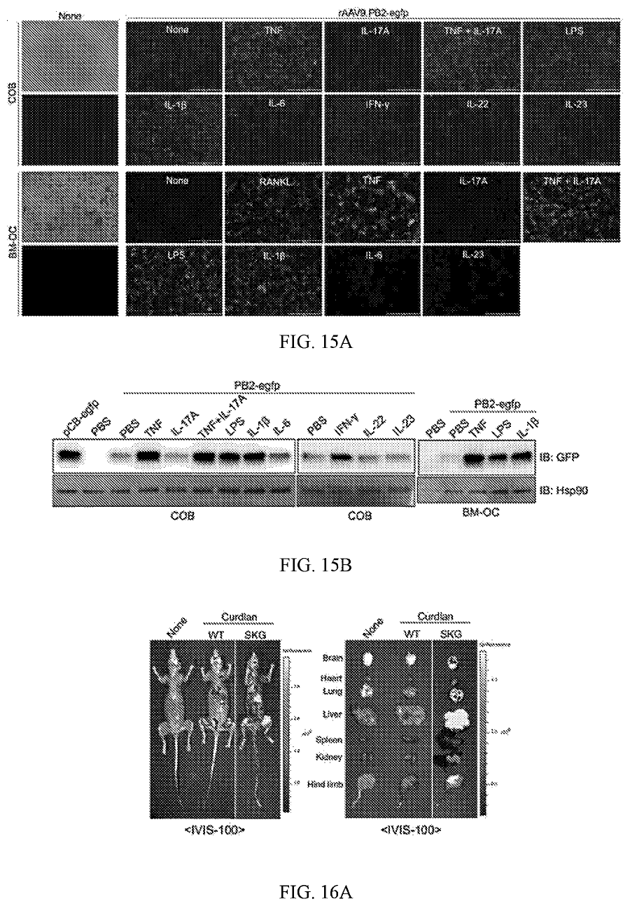

Next, to examine the responsiveness of PB2-egfp-expressing OBs/OCs to pro-inflammatory cytokines, the AAV genome vector containing the PB2-GFP cassette was packaged into AAV9 capsid and the vectors were transduced into primary COBs and BM-OCs. Similar to PB2-egfp-expressing HEK293 cells, EGFP expression in COBs was markedly upregulated by strong activators of the NF-κB pathway, including TNF, TNF+IL-17A, LPS, and IL-1β while IFN-γ induced a modest expression. However, little to no induction was detected in the presence of IL-17A, IL-6, IL-22, and IL-23, weak inducers of NF-κB activation (FIG. 15). Likewise, PB2-egfp-expressing BM-OCs show a high induction of EGFP when stimulated with RANKL, TNF, TNF+IL-17A, LPS, and IL-1(3, but not IL-17A, IL-6, and IL-23 (FIG. 15). These results indicate that EGFP induction in PB2-egfp-expressing OBs and OCs is corresponding to NF-κB activation by pro-inflammatory cytokines.

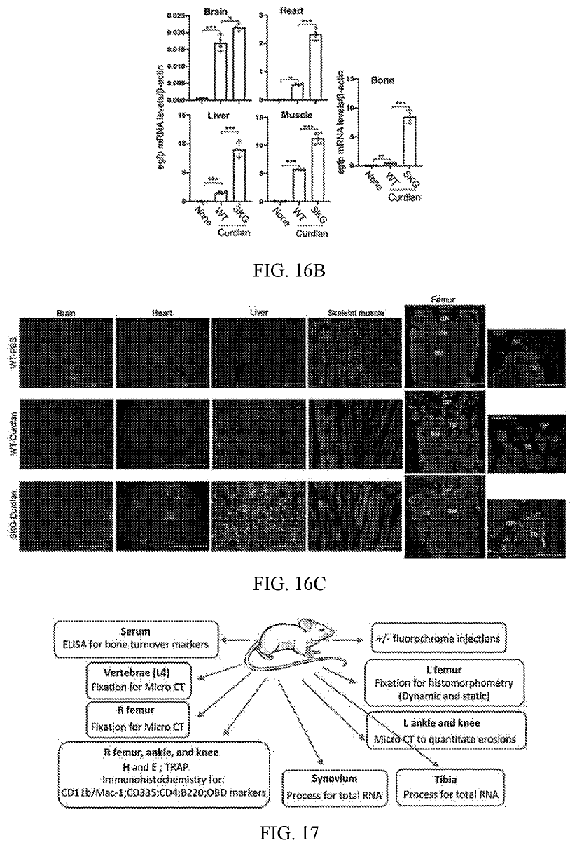

Onset of disease in SKG mice can be timed with systemic injection of curdlan via upregulation of NF-κB activating cytokines in synovium, including TNF, IL-17, IL-1 and IL-6. To test whether PB2-egfp-expressing tissues can induce EGFP expression in the setting of inflammatory arthritis, WT or SKG mice were treated with curdlan 2 weeks post-injection of rAAV9.PB2-egfp, and 3 weeks later, EGFP expression in whole body and individual tissues was assessed by optical imaging (FIG. 16A) and qPCR analysis (FIG. 16B). While little to no expression was detected in PBS-treated WT mice (None), curdlan-treated WT mice show a modest expression of EGFP in the brain, heart, liver, and skeletal muscle. Compared to these mice, EGFP expression in the heart, liver, skeletal muscle, and femur was markedly increased curdlan-treated SKG mice. This is consistent with histology data showing a significant increase in EGFP expression in cryo-sectioned heart, liver, skeletal muscle, and femur of SKG mice when treated with curdlan (FIG. 16C).

Given that rAAV9.PB2-egfp vector can express EGFP in non-skeletal tissues in the setting of inflammatory arthritis, complementary miR-1 and miR-122-binding sites will be inserted in the 3′-UTR of EGFP transgene and then, the AAV genome will be packaged into the bone-targeting AAV9 capsid (FIG. 12; Construct 4=DSS.rAAV9.PB2-egfp.MIR.BS). This technology allows the rAAV9.PB2-egfp vector to express a transgene only in the bone tissue when inflammation occurs. Additionally, to suppress joint inflammation, EGFP transgene was be replaced with sTNFR2 or sIL1Rα and then, the AAV genome was be packaged into DSS.rAAV9 capsid (FIG. 12; Construct 5=DSS.rAAV9.PB2-sTNFR2.MIR.BS, DSS.rAAV9.PB2-sIL1Rα.MIR.BS). To compare therapeutic effectiveness of inflammation-responsive expression and constitutive expression of sTNFR2 or sIL1Rα in inflammatory arthritis, the AAV genome vector that contains the CB promoter-driven expression of sTNFR2 or sIL1Rα and complementary miR-1 and miR-122-binding sites in the 3′-UTR of these transgenes were also generated (FIG. 12; Construct 6=DSS.rAAV9.CB-sTNFR2.MIR.BS, DSS.rAAV9.CB-sIL1Rα.MIR.BS). The amiR-ctrl, amiR-SHN3, or amiR-CTSK will be inserted intronically between the CB- or PB2-promoter and sTNFR2 or sIL1Rαtransgene, which can simultaneously suppress inflammation and prevent bone loss (FIG. 12; Construct 7=DSS.rAAV9.CB.amiR-ctrl, amiR-SHN3, amiR-CTSK.sTNFR2.MIR.BS, DSS.rAAV9.CB.amiR-ctrl, amiR-SHN3, amiR-CTSK.sIL1Rα.MIR.BS; FIG. 12; Construct 8=DSS.rAAV9.PB2.amiR-ctrl, amiR-SHN3, amiR-CTSK.sTNFR2.MIR.BS, DSS.rAAV9.PB2.amiR-ctrl, amiR-SHN3, amiR-CTSK.sIL1Rα.MIR.BS).

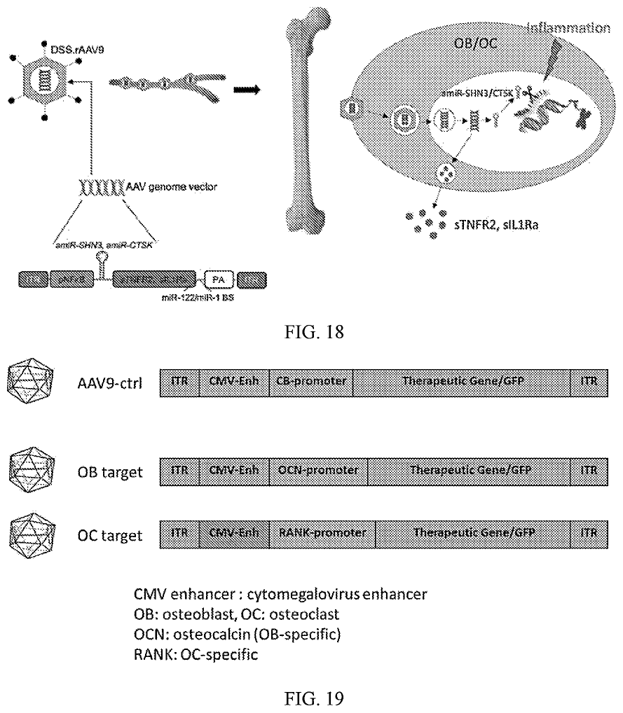

To examine their ability to suppress systemic and/or joint inflammation, systemic bone loss, and/or focal articular bone erosion in the setting of inflammatory arthritis, a single dose of 4×10¹¹ GC of the vectors is i.v. injected into the SKG model of inflammatory arthritis. 2 weeks later, SKG mice are treated with curdlan to accelerate inflammatory arthritis and euthanized 6 weeks after curdlan injection. Mice will be scored weekly for clinical inflammation using validated scoring systems that include joint swelling, caliper measurements of ankle thickness and grip strength. MicroCT imaging of knee and ankle joints for quantitation of erosion are performed, along with histologic assessments of articular erosion. Synovial mRNA is collected for quantitative PCR (qPCR) and serum ELISA assays measure cytokine expression levels (FIG. 18).

Systemic bone loss is quantified by measuring trabecular bone mass and cortical thickness of long bones and lumber vertebrae using microCT (FIG. 18). To quantify transduction efficiency and determine tissue distribution of DSS.rAAV9 vectors, EGFP expression in whole body and individual tissues is monitored by IVIS-100 optical imaging and fluorescence microscopy on cryo-sectioned tissues. EGFP expression in tissue extracts is also validated by immunoblotting with anti-GFP Ab. Alternatively, knockdown efficiency of SHN3 or CTSK and/or expression of sTNFR2 or sIL1Rα are examined by measuring mRNA levels in the tibial bone RNA (FIG. 18).

In vivo effects of the AAV vectors on OB and OC differentiation are assessed in H and E stained paraffin sections of femurs and joints (knee/ankle). Tartrate-resistant acid phosphatase (TRAP) are used as an OC marker, and slides are immunostained with the type I Collagen al (Coll) and Runx2 Abs as OB differentiation markers (OBD). Immune cell infiltration in joints is assessed by immunohistochemistry (IHC) for CD11b/Mac-1 (macrophage/monocyte), CD335 (NK cell), CD4 (T cell), and B220 (B cell) to confirm no difference in inflammation between groups (FIG. 18).

Dynamic and static histomorphometry are performed to analyze numbers of OB s and OCs, mineral apposition rates and mineralized surface/bone surface to calculate bone formation rates in femurs and joints. Mice are treated with intraperitoneal (i.p.) injections of calcein or alizarin labels five days apart to allow incorporation of these labels into bone (FIG. 18).

qPCR analysis of total long bone RNA are used to analyze expression of OB marker genes (e.g., tissue nonspecific alkaline phosphatase (TNALP), osteopontin (OPN), bone sialoprotein (BSP), osterix (OSX), osteocalcin (OCN)). Systemic OB and OC activity are analyzed by measuring serum bone turnover markers, including N-terminal propeptide of type 1 procollagen (P1NP) and type I collagen C-terminal telopeptide (CTX), respectively. FIG. 18 is a schematic showing one embodiment of molecular mechanisms related to the gene expression constructs described herein.

### Example 2: Design of Gene Expression Constructs

Additional expression constructs, which comprise either OC-specific promoters or OB-specific promoters were produced (FIG. 19). The OC-specific promoter included in the constructs comprises a RANK promoter. The OB-specific promoter comprises an osteocalcin (OCN) promoter.

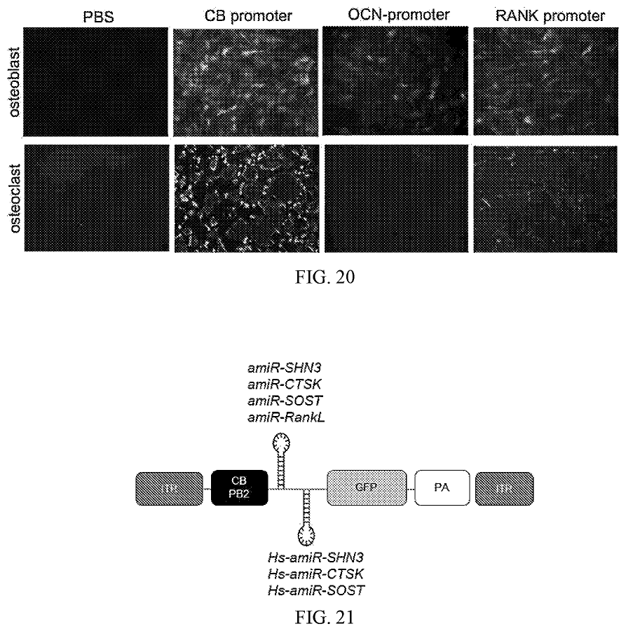

To test the ability of RANK or OCN promoter to drive the expression of GFP protein in OB- or OC-specific manner, rAAV9s with GFP expression driven by the CB, OCN, RANK promoter were incubated with COB or BM-OC and then differentiated into mature osteoblasts or osteoclasts, respectively. While OCN promoter was specific to express GFP protein in mature osteoblasts, CB and RANK promoter expressed GFP protein in both mature osteoblasts and osteoclasts (FIG. 20).

Dual expression constructs were also produced. One embodiment of a dual expression construct is shown in FIG. 21. The artificial microRNAs (amiRNAs) of these constructs comprise a miR-33 backbone. Note that the GFP protein shown in FIG. 21 may be replaced with a therapeutic protein, for example TNFR2, sIL1Rα, etc.

Sequences

In some embodiments, an isolated nucleic acid or vector (e.g., rAAV vector) described by the disclosure comprises or consists of a sequence set forth in any one of SEQ ID NOs: 1-40. In some embodiments, an isolated nucleic acid or vector (e.g., rAAV vector) described by the disclosure comprises or consists of a sequence that is complementary (e.g., the complement of) a sequence set forth in any one of SEQ ID NOs: 1-40. In some embodiments, an isolated nucleic acid or vector (e.g., rAAV vector) described by the disclosure comprises or consists of a sequence that is a reverse complement of a sequence set forth in any one of SEQ ID NOs: 1-40. In some embodiments, an isolated nucleic acid or vector (e.g., rAAV vector) described by the disclosure comprises or consists of a portion of a sequence set forth in any one of SEQ ID NOs: 1-40. A portion may comprise at least 25%, 50%, 60%, 70%, 80%, 90%, 95%, or 99% of a sequence set forth in any one of SEQ ID NOs: 1-40. In some embodiments, a nucleic acid sequence described by the disclosure is a nucleic acid sense strand (e.g., 5′ to 3′ strand), or in the context of a viral sequences a plus (+) strand. In some embodiments, a nucleic acid sequence described by the disclosure is a nucleic acid antisense strand (e.g., 3′ to 5′ strand), or in the context of viral sequences a minus (−) strand.

>CMV Enhancer nucleic acid sequence
(SEQ ID NO: 1)
TACGGTAAATGGCCCGCCTGGCTGACCGCCCAACGACCCCGCCCATTGACGTCAAT
AATGACGTATGTTCCCATAGTAACGCCAATAGGGACTTTCCATTGACGTCAATGGGT
GGAGTATTTACGGTAAACTGCCCACTTGGCAGTACATCAAGTGTATCATATGCCAA
GTACGCCCCCTATTGACGTCAATGACGGTAAATGGCCCGCCTGGCATTATGCCCAGT
ACATGACCTTATGGGACTTTCCTACTTGGCAGTACATCTACTCGAGGCCACGTTCTG
CTT
>Chicken beta-actin (CB) promoter nucleic acid sequence
(SEQ ID NO: 2)
TCTCCCCATCTCCCCCCCCTCCCCACCCCCAATTTTGTATTTATTTATTTTTTAATTAT
TTTGTGCAGCGATGGGGGCGGGGGGGGGGGGGGGGGGGGCGCGCGCCAGGCGGGG
CGGGGCGGGGCGAGGGGGGGGGCGGGGCGAGGCGGAGAGGTGCGGCGGCAGCCA
ATCAGAGCGGCGCGCTCCGAAAGTTTCCTTTTATGGCGAGGCGGCGGCGGCGGCGG
CCCTATAAAAAGCGAAGCGCGCGGCGGGCGGGAGCGGGATC
>PB2 (NF-kappa B (NFKB)) promoter nucleic acid sequence
(SEQ ID NO: 3)
TGAGCTCACAGAGGGGACTTTCCGAGAGATCTACAGAGGGGACTTTCCGAGAGCGA
GCTTGGGCTGCAGGTCGACCGTCCATCCATTCACAGCGCTTCTATAAAGGCGCCAGC
TGAGGCGCCTACTACTCCAACCGCGACTGCAGCGAGCAACTGAGAAGACTGGATAG
AGCCGGCGGTTCCGCGAACGAGCAGTGACCGCGCTCCCACCCAGCTCTGCTCTGCA
GCTCCACCAGTGTCTCTCTAGA
>Osteocalcin (OCN) promoter nucleic acid sequence
(SEQ ID NO: 4)
GAATTGCTCATCgcagcctgacggcgtaggttcatttcatttccacctagagcaagtgactgtaacgacacatttctttttgaatgT
TCTCATTCATGAAAGGCCTTCACTTTAATATTATTAcGAACATCTAATTTGTGGACAT
CAAGCGGGCTCCTCACACTCTGAAACCAGATACCCCCGAGCCCCCGCAGGTTTTTCT
TCCTCATTATCAGGGGTCCCAGGCATCTGGAGCTTAATGTGGGATGTCTCCAACACA
AGCAGGGCTAGAACCTCAAGGCAGAAGgTTGATGTTGAAGCTATAGAGGGGTGCTA
CTTACTGACTTGCTCATCATGGCATTCTCAGCCTGCTGTCTTATAGAAcCCAAGACCA
GGCCCAGGGATGGTAGCTCCCACAATGGGCTAGGCTCTTCCCCACCAACCACAAGA
AATGCCCTACAACCGGATcTTATGGAGGcATTTTCTCAATTGAGGITTTCTCCTTCCAA
GTTGACATAAAACTAACCAGACACTCCCCCCCaACACACACACacaCCCACTGGATGA
GCAGAGCTGCCCTGAACTGGGCAAATGAGGACATTACTGAaCACTCCCTCCCTGGG
GTTTGGCTCCCGCTCTCAGGGGCAGACACTGAAAATCACAGGCTATGAGAGTTGGA
GCCCAGTTTATCCCAAACCGATTTTAGATCTCTGTACCATGTCTAGGCTATGCaTAGG
GUTCTTGTCTCTAGGGCgACCCAGTGCTCCAGCTGAGGCTGAGAGAGAGAGagCAcacA
TAGGAGTggTGGAGCAGCCCCTCAGGGAAGAGGTCTgGGGCCATGTCAGAgCCTGGC
AGTCTCCgATTGTGGCCTCTCGTCCACTCCCAGAGCCTTGCCCAGGCAGCIGCAATCA
CCaACCACAGCATCCTTTGGGTTTGACCCACTGAGCACATGACCCCCAATTAGTCCT
GGCAGCATCCCCTGCTCCTCCTGCTTACATCAGAGAGCACAGAgTAGCcGATATAAA
TGCTACTGGAtGCTGGAGGGTGCAG
>RANK promoter nucleic acid sequence
(SEQ ID NO: 5)
GAAAGGGTTTTGTAACCTGAAAATTTTCCCACTTTACTGTTTTAAAAATAATATCAT
TTTAAATCAAAGAGTTCTACTGTAGTATTTTATTTAACACAAAATTGTATTGCAATG
CATGAAATAAAATACTGTTTCCAAAATTGTTTTAGAGAAACACAAAGGAAGTCAAT
AATAAAAGTGGACTAAAGATTCAACAAACAGAAAAAATCAGAAAGGTAATAGACT
TCAATTCACCGTATGTGACGATCACTAAATGTGCACGCTCCAATAGGAAGGCAGGA
ACAGTCTAAAATGTATGCTGCCACTTTAAACAGTCTTGCATGGGAAAGAATCAGAT
ATACTACTTACATAGCATTCATAAGAAAGGTGACATGGCTACACATAAATGTAAAG
TAGACCAGAATGTGAAATTTTATGTTGATCAAGAAGGGCATGTTGTGATGATAAAA
GGTCAATGTACCGAGAAGACATAAATCGCTATGTCTCAAGGGAGTTTCAAGGATGA
GAAAAACTGACAGAATTCAAAGAAATAATAGACAAATCCAGTCTTCCTTGTTAGAA
AACTCCATAGTTTCTCAGAATTAGACGGAAAAACCATTAACATGGAGGTCAGAAAG
CTGGCAGAGACGGTTCTGGCACTCAAGGAATTGAAAACACTGTGTTTCTGTTGACTG
GGAGACAAAGCTCTTCCTCTTACTGTTACTACACAGAGGCAAGGCTGTATGTACCAT
TGTGTAACCAACACTGACCTGAAATTTGGAAAAAGACAAACCCAGAAGCCAGTAAT
GAGAATCCCTTAACTAATACGACAGGATCGCTTATTCCTGACTTTAAACTAGTTCTG
TATTAGAAGAGGTAAGGTAGGTGACCTAGCGGTTCGGGTGAAGGGCTTCGTTAAGC
ATGGTGGCAAGTTTCTACCTACCTGGAAAGCTTGCTATGAGTGTTACAGAGGGGGT
GAAGGGCAGAGACAGGTGCGGTGACTCGGAGAACAGCCAAGAGGAAGCGGTTGTT
GGAGGCGGCCAGAATGAGGGGAAGTATAGGGCTGGGTAAAGGAGGTTTGGGGGAG
CCGTGGACACTACATTGTGTAGATTCTTTAGTTACAGTTAAGGAAACCCCAACAGG
GGTGCACCTTGTGCAGGCAGGGTCCACAGTGGCGACAAGGCAGGGTCCACTGAAGG
CGAAGGACAACCTTGGCAGGGCTATCTAGCGCCTGCACGCAGGTGCTGCACCCAGA
GAGCTCAGAGCCTGGGGACACTCGGACAGGACTCCGCGCGGGTCTGAAGCACTCGT
GGAATGCCAAAACCATCTCTGTCCCGCGTCACGGCAGCCCACGCTCGGGCACCCCC
TGGCGGAGCTGGTCGGCGGCGCGGGGGCAGGTGCCGGGCGGAGCCGGGCGCACGG
GGCGGGACGAGGCGGGCGGAGGGCGGCGGCGACCGCCGGTCCACAGAGGCCGCGC
GCCCAGCCCGCCCGCACCGCGCCAT
>Soluble TNFα Receptor 2 (sTNFR2) nucleic acid sequence
(SEQ ID NO: 6)
ATGGCGCCCGTCGCCGTCTGGGCCGCGCTGGCCGTCGGACTGGAGCTCTGGGCTGC
GGCGCACGCCTTGCCCGCCCAGGTGGCATTTACACCCTACGCCCCGGAGCCCGGGA
GCACATGCCGGCTCAGAGAATACTATGACCAGACAGCTCAGATGTGCTGCAGCAAA
TGCTCGCCGGGCCAACATGCAAAAGTCTTCTGTACCAAGACCTCGGACACCGTGTG
TGACTCCTGTGAGGACAGCACATACACCCAGCTCTGGAACTGGGTTCCCGAGTGCTT
GAGCTGTGGCTCCCGCTGTAGCTCTGACCAGGTGGAAACTCAAGCCTGCACTCGGG
AACAGAACCGCATCTGCACCTGCAGGCCCGGCTGGTACTGCGCGCTGAGCAAGCAG
GAGGGGTGCCGGCTGTGCGCGCCGCTGCGCAAGTGCCGCCCGGGCTTCGGCGTGGC
CAGACCAGGAACTGAAACATCAGACGTGGTGTGCAAGCCCTGTGCCCCGGGGACGT
TCTCCAACACGACTTCATCCACGGATATTTGCAGGCCCCACCAGATCTGTAACGTGG
TGGCCATCCCTGGGAATGCAAGCATGGATGCAGTCTGCACGTCCACGTCCCCCACC
CGGAGTATGGCCCCAGGGGCAGTACACTTACCCCAGCCAGTGTCCACACGATCCCA
ACACACGCAGCCAACTCCAGAACCCAGCACTGCTCCAAGCACCTCCTTCCTGCTCCC
AATGGGCCCCAGCCCCCCAGCTGAAGGGAGCACTGGCGACTTCGCTCTTCCAGTT
>Soluble TNFα Receptor 2 (sTNFR2) amino acid sequence
(SEQ ID NO: 7)
MAPVAVWAALAVGLELWAAAHALPAQVAFTPYAPEPGSTCRLREYYDQTAQMCCSK
CSPGQHAKVFCTKTSDTVCDSCEDSTYTQLWNWVPECLSCGSRCSSDQVETQACTREQ
NRICTCRPGWYCALSKQEGCRLCAPLRKCRPGFGVARPGTETSDVVCKPCAPGTFSNTT
SSTDICRPHQICNVVAIPGNASMDAVCTSTSPTRSMAPGAVHLPQPVSTRSQHTQPTPEP
STAPSTSFLLPMGPSPPAEGSTGDFALPV
>hs-amiRNA33-mSHN3 nucleic acid sequence
(SEQ ID NO: 8)
gatctGGCAGCCTTGGAGTGGGTTCCTGCCCCCTCGGGCACACAAACAGAGCTGAAGA
CCACCCTGGGCACCTCCTTGGCTGGCCGCATACCTCCTGGCGGGCAGCTGTGtacaaact
acttgagagcaggTGTTCTGGTGGTACCCAcctgctctgtaatagtttgtaCACAGAGGCCTGCCTGGCC
CTCGAGAGACTGCCCTGACTGAAGGCCCTATCAGGTGGGGGAGGGGATCCTGATAG
AGGGCACTGCTGCCACTGTTGGGGCCCAAGctgca
>hs-amiRNA33-hSHN3 nucleic acid sequence
(SEQ ID NO: 9)
GGCAGCCTTGGAGTGGGTTCCTGCCCCCTCGGGCACACAAACAGAGCTGAAGACCA
CCCTGGGCACCTCCTTGGCTGGCCGCATACCTCCTGGCGGGCAGCTGTGtttcatcatagtac
acacctcTGTTCTGGTGGTACCCAGAGGTGTGATCCATGATGAAACACAGAGGCCTGCC
TGGCCCTCGAGAGACTGCCCTGACTGAAGGCCCTATCAGGTGGGGGAGGGGATCCT
GATAGAGGGCACTGCTGCCACTGTTGGGGCCCAAG
>amiR-33-mSHN3 nucleic acid sequence
(SEQ ID NO: 10)
tttgtcttttatttcaggtcccAGATCTAGGGCTCTGCGTTTGCTCCAGGTAGTCCGCTGCTCCC
TTGGGCCTGGGCCCACTGACAGCCCTGGTGCCTCTGGCCGGCTGCACACCTCCTG
GCGGGCAGCTGTGTACAAACTACTTGAGAGCAGGTGTTCTGGCAATACCTGCCTG
CTCTGTAATAGTTTGTACACGGAGGCCTGCCCTGACTGCCCACGGTGCCGTGGCC
AAAGAGGATCTAAGGGCACCGCTGAGGGCCTACCTAACCATCGTGGGGAATAAGG
ACAGTGTCACCCCTGCAGgggatccggtggtggtgcaaatca
>amiR-33-hSHN3 nucleic acid sequence
(SEQ ID NO: 11)
gtcttttatttcaggtcccagatcttAGGGCTCTGCGTTTGCTCCAGGTAGTCCGCTGCTCCCTTGGGC
CTGGGCCCACTGACAGCCCTGGTGCCTCTGGCCGGCTGCACACCTCCTGGCGGGCA
GCTGTGtttccatggtaagttcaaggcTGTTCTGGCAATACCTGGCCTTGAAGATGCCATGGAAA
CACGGAGGCCTGCCCTGACTGCCCACGGTGCCGTGGCCAAAGAGGATCTAAGGGCA
CCGCTGAGGGCCTACCTAACCATCGTGGGGAATAAGGACAGTGTCACCCccctgcagggg
atccggtggtggtgcaaat
>amiR-mRankL nucleic acid sequence 1
(SEQ ID NO: 12)
AGGGCTCTGCGTTTGCTCCAGGTAGTCCGCTGCTCCCTTGGGCCTGGGCCCACTGAC
AGCCCTGGTGCCTCTGGCCGGCTGCACACCTCCTGGCGGGCAGCTGTGtaacgtcatgttaga
gatcttTGTTCTGGCAATACCTGAAGATCTCATATATGACGTTACACGGAGGCCTGCCCT
GACTGCCCACGGTGCCGTGGCCAAAGAGGATCTAAGGGCACCGCTGAGGGCCTACC
TAACCATCGTGGGGAATAAGGACAGTGTCACCC
>hs-amiR-mRankL nucleic acid sequence 1
(SEQ ID NO: 13)
GGCAGCCTTGGAGTGGGTTCCTGCCCCCTCGGGCACACAAACAGAGCTGAAGACCA
CCCTGGGCACCTCCTTGGCTGGCCGCATACCTCCTGGCGGGCAGCTGTGtaacgtcatgttag
agatcttTGTTCTGGTGGTACCCAAAGATCTCATATATGACGTTACACAGAGGCCTGCCT
GGCCCTCGAGAGACTGCCCTGACTGAAGGCCCTATCAGGTGGGGGAGGGGATCCTG
ATAGAGGGCACTGCTGCCACTGTTGGGGCCCAAG
>amiR-mRankL nucleic acid sequence 2
(SEQ ID NO: 14)
AGGGCTCTGCGTTTGCTCCAGGTAGTCCGCTGCTCCCTTGGGCCTGGGCCCACTGAC
AGCCCTGGTGCCTCTGGCCGGCTGCACACCTCCTGGCGGGCAGCTGTGacaggtaatagaag
ccatcttTGTTCTGGCAATACCTGAAGATGGCAACCATTACCTGTCACGGAGGCCTGCCC
TGACTGCCCACGGTGCCGTGGCCAAAGAGGATCTAAGGGCACCGCTGAGGGCCTAC
CTAACCATCGTGGGGAATAAGGACAGTGTCACCC
>hs-amiR-mRankL nucleic acid sequence 2
(SEQ ID NO: 15)
GGCAGCCTTGGAGTGGGTTCCTGCCCCCTCGGGCACACAAACAGAGCTGAAGACCA
CCCTGGGCACCTCCTTGGCTGGCCGCATACCTCCTGGCGGGCAGCTGTGacaggtaataga
agccatcttTGTTCTGGTGGTACCCAAAGATGGCAACCATTACCTGTCACAGAGGCCTGC
CTGGCCCTCGAGAGACTGCCCTGACTGAAGGCCCTATCAGGTGGGGGAGGGGATCC
TGATAGAGGGCACTGCTGCCACTGTTGGGGCCCAAG
>amiR-hRankL nucleic acid sequence 1
(SEQ ID NO: 16)
AGGGCTCTGCGTTTGCTCCAGGTAGTCCGCTGCTCCCTTGGGCCTGGGCCCACTGAC
AGCCCTGGTGCCTCTGGCCGGCTGCACACCTCCTGGCGGGCAGCTGTGtttagtgacgtacac
cattagTGTTCTGGCAATACCTGCTAATGGTCAATGTCACTAAACACGGAGGCCTGCCC
TGACTGCCCACGGTGCCGTGGCCAAAGAGGATCTAAGGGCACCGCTGAGGGCCTAC
CTAACCATCGTGGGGAATAAGGACAGTGTCACCC
>amiR-hRankL nucleic acid sequence 2
(SEQ ID NO: 17)
AGGGCTCTGCGTTTGCTCCAGGTAGTCCGCTGCTCCCTTGGGCCTGGGCCCACTGAC
AGCCCTGGTGCCTCTGGCCGGCTGCACACCTCCTGGCGGGCAGCTGTGatccaccatcgcttt
ctctgcTGTTCTGGCAATACCTGGCAGAGAATCCAATGGTGGATCACGGAGGCCTGCCC
TGACTGCCCACGGTGCCGTGGCCAAAGAGGATCTAAGGGCACCGCTGAGGGCCTAC
CTAACCATCGTGGGGAATAAGGACAGTGTCACCC
>hs-amiR-hRankL nucleic acid sequence 1
(SEQ ID NO: 18)
GGCAGCCTTGGAGTGGGTTCCTGCCCCCTCGGGCACACAAACAGAGCTGAAGACCA
CCCTGGGCACCTCCTTGGCTGGCCGCATACCTCCTGGCGGGCAGCTGTGtttagtgacgtaca
ccattagTGTTCTGGTGGTACCCACTAATGGTCAATGTCACTAAACACAGAGGCCTGCCT
GGCCCTCGAGAGACTGCCCTGACTGAAGGCCCTATCAGGTGGGGGAGGGGATCCTG
ATAGAGGGCACTGCTGCCACTGTTGGGGCCCAAG
>hs-amiR-hRankL nucleic acid sequence 2
(SEQ ID NO: 19)
GGCAGCCTTGGAGTGGGTTCCTGCCCCCTCGGGCACACAAACAGAGCTGAAGACCA
CCCTGGGCACCTCCTTGGCTGGCCGCATACCTCCTGGCGGGCAGCTGTGatccaccategct
ttctctgcTGTTCTGGTGGTACCCAGCAGAGAATCCAATGGTGGATCACAGAGGCCTGCC
TGGCCCTCGAGAGACTGCCCTGACTGAAGGCCCTATCAGGTGGGGGAGGGGATCCT
GATAGAGGGCACTGCTGCCACTGTTGGGGCCCAAG
>amiRNA-mCathepsin K nucleic acid sequence
(SEQ ID NO: 20)
catggGGCAGCCTTGGAGTGGGTTCCTGCCCCCTCGGGCACACAAACAGAGCTGAAGA
CCACCCTGGGCACCTCCTTGGCTGGCCGCATACCTCCTGGCGGGCAGCTGTGtttcatcat
agtacacacctcTGTTCTGGTGGTACCCAgaggtgtgatccatgatgaaaCACAGAGGCCTGCCTGGCC
CTCGAGAGACTGCCCTGACTGAAGGCCCTATCAGGTGGGGGAGGGGATCCTGATAG
AGGGCACTGCTGCCACTGTTGGGGCCCAAGaagct
>hs-amiRNA-mCathepsin K nucleic acid sequence
(SEQ ID NO: 21)
gtcttttatttcaggtcccagatctGGCAGCCTTGGAGTGGGTTCCTGCCCCCTCGGGCACACAAAC
AGAGCTGAAGACCACCCTGGGCACCTCCTTGGCTGGCCGCATACCTCCTGGCGGGC
AGCTGTGtttcatcatagtacacacctcTGTTCTGGTGGTACCCAgaggtgtgatccatgatgaaaCACAGAG
GCCTGCCTGGCCCTCGAGAGACTGCCCTGACTGAAGGCCCTATCAGGTGGGGGAGG
GGATCCTGATAGAGGGCACTGCTGCCACTGTTGGGGCCCAAGctgcaggggatccggtggtggt
gc
>amiRNA-hCathepsin K nucleic acid sequence 1
(SEQ ID NO: 22)
gtcttttatttcaggtcccagatcttAGGGCTCTGCGTTTGCTCCAGGTAGTCCGCTGCTCCCTTGGGC
CTGGGCCCACTGACAGCCCTGGTGCCTCTGGCCGGCTGCACACCTCCTGGCGGGCA
GCTGTGattatcgctattgcagctttcTGTTCTGGCAATACCTGGAAAGCTGGTACAGCGATAATC
ACGGAGGCCTGCCCTGACTGCCCACGGTGCCGTGGCCAAAGAGGATCTAAGGGCAC
CGCTGAGGGCCTACCTAACCATCGTGGGGAATAAGGACAGTGTCACCC
ccctgcaggggatccggtggtggtgcaaat
>amiRNA-hCathepsin K nucleic acid sequence 2
(SEQ ID NO: 23)
gtcttttatttcaggtcccagatcttAGGGCTCTGCGTTTGCTCCAGGTAGTCCGCTGCTCCCTTGGGC
CTGGGCCCACTGACAGCCCTGGTGCCTCTGGCCGGCTGCACACCTCCTGGCGGGCA
GCTGTGtcagattatcgctattgcagcTGTTCTGGCAATACCTGGCTGCAATTCCAATAATCTGAC
ACGGAGGCCTGCCCTGACTGCCCACGGTGCCGTGGCCAAAGAGGATCTAAGGGCAC
CGCTGAGGGCCTACCTAACCATCGTGGGGAATAAGGACAGTGTCACCC
ccctgcaggggatccggtggtggtgcaaat
>hs-amiRNA-hCathepsin K nucleic acid sequence 1
(SEQ ID NO: 24)
GGCAGCCTTGGAGTGGGTTCCTGCCCCCTCGGGCACACAAACAGAGCTGAAGACCA
CCCTGGGCACCTCCTTGGCTGGCCGCATACCTCCTGGCGGGCAGCTGTGattatcgctattgc
agctttcTGTTCTGGTGGTACCCAGAAAGCTGGTACAGCGATAATCACAGAGGCCTGCC
TGGCCCTCGAGAGACTGCCCTGACTGAAGGCCCTATCAGGTGGGGGAGGGGATCCT
GATAGAGGGCACTGCTGCCACTGTTGGGGCCCAAG
>hs-amiRNA-hCathepsin K nucleic acid sequence 2
(SEQ ID NO: 25)
GGCAGCCTTGGAGTGGGTTCCTGCCCCCTCGGGCACACAAACAGAGCTGAAGACCA
CCCTGGGCACCTCCTTGGCTGGCCGCATACCTCCTGGCGGGCAGCTGTGtcagattategcta
ttgcagcTGTTCTGGTGGTACCCAGCTGCAATTCCAATAATCTGACACAGAGGCCTGCCT
GGCCCTCGAGAGACTGCCCTGACTGAAGGCCCTATCAGGTGGGGGAGGGGATCCTG
ATAGAGGGCACTGCTGCCACTGTTGGGGCCCAAG
>amiRNA-mSOST nucleic acid sequence
(SEQ ID NO: 26)
gatctagggctctgcgtttgctccaggtagtccgctgctcccttgggcctgggcccactgacagccctggtgcctctggccggctgcacac
ctcctggcgggcagctgtgtgacctctgtggcatcattcctgttctggcaatacctgggaatgatcgcgcagaggtcacacggaggcctgc
cctgactgcccacggtgccgtggccaaagaggatctaagggcaccgctgagggcctacctaaccatcgtggggaataaggacagtgtca
cccctgca
>hs-amiRNA-mSOST nucleic acid sequence
(SEQ ID NO: 27)
GGCAGCCTTGGAGTGGGTTCCTGCCCCCTCGGGCACACAAACAGAGCTGAAGACCA
CCCTGGGCACCTCCTTGGCTGGCCGCATACCTCCTGGCGGGCAGCTGTGtgacctctgtggc
atcattccTGTTCTGGTGGTACCCAGGAATGATCGCGCAGAGGTCACACAGAGGCCTGCC
TGGCCCTCGAGAGACTGCCCTGACTGAAGGCCCTATCAGGTGGGGGAGGGGATCCT
GATAGAGGGCACTGCTGCCACTGTTGGGGCCCAAG
>amiRNA-hSOST nucleic acid sequence 1
(SEQ ID NO: 28)
gtcttttatttcaggtcccagatcttAGGGCTCTGCGTTTGCTCCAGGTAGTCCGCTGCTCCCTTGGGC
CTGGGCCCACTGACAGCCCTGGTGCCTCTGGCCGGCTGCACACCTCCTGGCGGGCA
GCTGTGatggtcttgttgttctccagcTGTTCTGGCAATACCTGGCTGGAGATGAGCAAGACCAT
CACGGAGGCCTGCCCTGACTGCCCACGGTGCCGTGGCCAAAGAGGATCTAAGGGCA
CCGCTGAGGGCCTACCTAACCATCGTGGGGAATAAGGACAGTGTCACCC
ccctgcaggggatccggtggtggtgcaaat
>amiRNA-hSOST nucleic acid sequence 2
(SEQ ID NO: 29)
gtcttttatttcaggtcccagatcttAGGGCTCTGCGTTTGCTCCAGGTAGTCCGCTGCTCCCTTGGGC
CTGGGCCCACTGACAGCCCTGGTGCCTCTGGCCGGCTGCACACCTCCTGGCGGGCA
GCTGTGACGtCtttGGtCtCAAAGGGGTGTTCTGGCAATACCTGCCCCTTTGTCATCAAA
GACGTCACGGAGGCCTGCCCTGACTGCCCACGGTGCCGTGGCCAAAGAGGATCTAA
GGGCACCGCTGAGGGCCTACCTAACCATCGTGGGGAATAAGGACAGTGTCACCC
ccctgcaggggatccggtggtggtgcaaat
>hs-amiRNA-hSOST nucleic acid sequence 1
(SEQ ID NO: 30)
GGCAGCCTTGGAGTGGGTTCCTGCCCCCTCGGGCACACAAACAGAGCTGAAGACCA
CCCTGGGCACCTCCTTGGCTGGCCGCATACCTCCTGGCGGGCAGCTGTGatggtcttgttgttc
tccagcTGTTCTGGTGGTACCCAGCTGGAGATGAGCAAGACCATCACAGAGGCCTGCC
TGGCCCTCGAGAGACTGCCCTGACTGAAGGCCCTATCAGGTGGGGGAGGGGATCCT
GATAGAGGGCACTGCTGCCACTGTTGGGGCCCAAG
>hs-amiRNA-hSOST nucleic acid sequence 2
(SEQ ID NO: 31)
GGCAGCCTTGGAGTGGGTTCCTGCCCCCTCGGGCACACAAACAGAGCTGAAGACCA
CCCTGGGCACCTCCTTGGCTGGCCGCATACCTCCTGGCGGGCAGCTGTGACGtCtttGG
tCtCAAAGGGGTGTTCTGGTGGTACCCACCCCTTTGTCATCAAAGACGTCACAGAGGC
CTGCCTGGCCCTCGAGAGACTGCCCTGACTGAAGGCCCTATCAGGTGGGGGAGGGG
ATCCTGATAGAGGGCACTGCTGCCACTGTTGGGGCCCAAG
>miR-122 binding site
(SEQ ID NO: 32)
ATACATACTTCTTTACATTCCAATACATACTTCTTTACATTCCAATACATACTTCTTT
ACATTCCACCATGGACTAGTACAAACACCATTGTCACACTCCAACAAACACCATTGT
CACACTCCAACAAACACCATTGTCACACTCCAA
>rAAVsc CB6 PI sTNFR2 miR1&122 BS
(SEQ ID NO: 33)
CTGCGCGCTCGCTCGCTCACTGAGGCCGCCCGGGCAAAGCCCGGGCGTCGGGCGAC
CTTTGGTCGCCCGGCCTCAGTGAGCGAGCGAGCGCGCAGAGAGGGAGTGTAGCCAT
GCTCTAGGAAGATCAATTCGGTACAATTCACGCGTCGACATTGATTATTGACTCTGG
TCGTTACATAACTTACGGTAAATGGCCCGCCTGGCTGACCGCCCAACGACCCCGCCC
ATTGACGTCAATAATGACGTATGTTCCCATAGTAACGCCAATAGGGACTTTCCATTG
ACGTCAATGGGTGGAGTATTTACGGTAAACTGCCCACTTGGCAGTACATCAAGTGT
ATCATATGCCAAGTACGCCCCCTATTGACGTCAATGACGGTAAATGGCCCGCCTGG
CATTATGCCCAGTACATGACCTTATGGGACTTTCCTACTTGGCAGTACATCTACTCG
AGGCCACGTTCTGCTTCACTCTCCCCATCTCCCCCCCCTCCCCACCCCCAATTTTGTA
TTTATTTATTTTTTAATTATTTTGTGCAGCGATGGGGGGGGGGGGGGGGGGGGGGGG
GGCGCGCGCCAGGCGGGGCGGGGCGGGGCGAGGGGCGGGGCGGGGCGAGGCGGA
GAGGTGCGGCGGCAGCCAATCAGAGCGGCGCGCTCCGAAAGTTTCCTTTTATGGCG
AGGCGGCGGCGGCGGCGGCCCTATAAAAAGCGAAGCGCGCGGCGGGCGGGAGCGG
GATCAGCCACCGCGGTGGCGGCCCTAGAGTCGATCGAGGAACTGAAAAACCAGAA
AGTTAACTGGTAAGTTTAGTCTTTTTGTCTTTTATTTCAGGTCCCAGATCTGTTTAAA
CCTGCAGGGGATCCGGTGGTGGTGCAAATCAAAGAACTGCTCCTCAGTGGATGTTG
CCTTTACTTCTAGGCCTGTACGGAAGTGTTACTTCTGCTCTAAAAGCTGCGGAATTG
TACCCGCGGCCGATCCACCGGTGCCACCATGGCGCCCGTCGCCGTCTGGGCCGCGC
TGGCCGTCGGACTGGAGCTCTGGGCTGCGGCGCACGCCTTGCCCGCCCAGGTGGCA
TTTACACCCTACGCCCCGGAGCCCGGGAGCACATGCCGGCTCAGAGAATACTATGA
CCAGACAGCTCAGATGTGCTGCAGCAAATGCTCGCCGGGCCAACATGCAAAAGTCT
TCTGTACCAAGACCTCGGACACCGTGTGTGACTCCTGTGAGGACAGCACATACACC
CAGCTCTGGAACTGGGTTCCCGAGTGCTTGAGCTGTGGCTCCCGCTGTAGCTCTGAC
CAGGTGGAAACTCAAGCCTGCACTCGGGAACAGAACCGCATCTGCACCTGCAGGCC
CGGCTGGTACTGCGCGCTGAGCAAGCAGGAGGGGTGCCGGCTGTGCGCGCCGCTGC
GCAAGTGCCGCCCGGGCTTCGGCGTGGCCAGACCAGGAACTGAAACATCAGACGTG
GTGTGCAAGCCCTGTGCCCCGGGGACGTTCTCCAACACGACTTCATCCACGGATATT
TGCAGGCCCCACCAGATCTGTAACGTGGTGGCCATCCCTGGGAATGCAAGCATGGA
TGCAGTCTGCACGTCCACGTCCCCCACCCGGAGTATGGCCCCAGGGGCAGTACACT
TACCCCAGCCAGTGTCCACACGATCCCAACACACGCAGCCAACTCCAGAACCCAGC
ACTGCTCCAAGCACCTCCTTCCTGCTCCCAATGGGCCCCAGCCCCCCAGCTGAAGGG
AGCACTGGCGACTTCGCTCTTCCAGTTTGATGTACAAGTAAGCGGCCGCATACATAC
TTCTTTACATTCCAATACATACTTCTTTACATTCCAATACATACTTCTTTACATTCCA
CCATGGACTAGTACAAACACCATTGTCACACTCCAACAAACACCATTGTCACACTCC
AACAAACACCATTGTCACACTCCAAAAGCTTATCGATACCGTCGACTAGAGCTCGC
TGATCAGCCTCGACTGTGCCTTCTAGTTGCCAGCCATCTGTTGTTTGCCCCTCCCCCG
TGCCTTCCTTGACCCTGGAAGGTGCCACTCCCACTGTCCTTTCCTAATAAAATGAGG
AAATTGCATCGCATTGTCTGAGTAGGTGTCATTCTATTCTGGGGGGTGGGGTGGGGC
AGGACAGCAAGGGGGAGGATTGGGAAGACAATTAGGTAGATAAGTAGCATGGCGG
GTTAATCATTAACTACAAGGAACCCCTAGTGATGGAGTTGGCCACTCCCTCTCTGCG
CGCTCGCTCGCTCACTGAGGCCGGGCGACCAAAGGTCGCCCGACGCCCGGGCTTTG
CCCGGGCGGCCTCAGTGAGCGAGCGAGCGCGCAG
>Soluble IL-1 Receptor Antagonist (sILIRα) nucleic acid sequence
(SEQ ID NO: 34)
ATGGAAATCTGCAGAGGCCTCCGCAGTCACCTAATCACTCTCCTCCTCTTCCTGTTC
CATTCAGAGACGATCTGCCGACCCTCTGGGAGAAAATCCAGCAAGATGCAAGCCTT
CAGAATCTGGGATGTTAACCAGAAGACCTTCTATCTGAGGAACAACCAACTAGTTG
CTGGATACTTGCAAGGACCAAATGTCAATTTAGAAGAAAAGATAGATGTGGTACCC
ATTGAGCCTCATGCTCTGTTCTTGGGAATCCATGGAGGGAAGATGTGCCTGTCCTGT
GTCAAGTCTGGTGATGAGACCAGACTCCAGCTGGAGGCAGTTAACATCACTGACCT
GAGCGAGAACAGAAAGCAGGACAAGCGCTTCGCCTTCATCCGCTCAGACAGTGGCC
CCACCACCAGTTTTGAGTCTGCCGCCTGCCCCGGTTGGTTCCTCTGCACAGCGATGG
AAGCTGACCAGCCCGTCAGCCTCACCAATATGCCTGACGAAGGCGTCATGGTCACC
AAATTCTACTTCCAGGAGGACGAGTAGTAA
>Soluble IL-1 Receptor Antagonist (sIL1Rα) amino acid sequence
(SEQ ID NO: 35)
MEICRGLRSHLITLLLFLFHSETICRPSGRKSSKMQAFRIWDVNQKTFYLRNNQLVAGYL
QGPNVNLEEKIDVVPIEPHALFLGIHGGKMCLSCVKSGDETRLQLEAVNITDLSENRKQ
DKRFAFIRSDSGPTTSFESAACPGWFLCTAMEADQPVSLTNMPDEGVMVTKFYFQEDE
>rAAVsc CB6 PI sIL-1Ra miR1&122 BS
(SEQ ID NO: 36)
CTGCGCGCTCGCTCGCTCACTGAGGCCGCCCGGGCAAAGCCCGGGCGTCGGGCGAC
CTTTGGTCGCCCGGCCTCAGTGAGCGAGCGAGCGCGCAGAGAGGGAGTGTAGCCAT
GCTCTAGGAAGATCAATTCGGTACAATTCACGCGTCGACATTGATTATTGACTCTGG
TCGTTACATAACTTACGGTAAATGGCCCGCCTGGCTGACCGCCCAACGACCCCGCCC
ATTGACGTCAATAATGACGTATGTTCCCATAGTAACGCCAATAGGGACTTTCCATTG
ACGTCAATGGGTGGAGTATTTACGGTAAACTGCCCACTTGGCAGTACATCAAGTGT
ATCATATGCCAAGTACGCCCCCTATTGACGTCAATGACGGTAAATGGCCCGCCTGG
CATTATGCCCAGTACATGACCTTATGGGACTTTCCTACTTGGCAGTACATCTACTCG
AGGCCACGTTCTGCTTCACTCTCCCCATCTCCCCCCCCTCCCCACCCCCAATTTTGTA
TTTATTTATTTTTTAATTATTTTGTGCAGCGATGGGGGCGGGGGGGGGGGGGGGGGG
GGCGCGCGCCAGGCGGGGCGGGGCGGGGCGAGGGGCGGGGCGGGGCGAGGCGGA
GAGGTGCGGCGGCAGCCAATCAGAGCGGCGCGCTCCGAAAGTTTCCTTTTATGGCG
AGGCGGCGGCGGCGGCGGCCCTATAAAAAGCGAAGCGCGCGGCGGGCGGGAGCGG
GATCAGCCACCGCGGTGGCGGCCCTAGAGTCGATCGAGGAACTGAAAAACCAGAA
AGTTAACTGGTAAGTTTAGTCTTTTTGTCTTTTATTTCAGGTCCCAGATCTGTTTAAA
CCTGCAGGGGATCCGGTGGTGGTGCAAATCAAAGAACTGCTCCTCAGTGGATGTTG
CCTTTACTTCTAGGCCTGTACGGAAGTGTTACTTCTGCTCTAAAAGCTGCGGAATTG
TACCCGCGGCCGATCCACCGGTGCCACCATGGAAATCTGCAGAGGCCTCCGCAGTC
ACCTAATCACTCTCCTCCTCTTCCTGTTCCATTCAGAGACGATCTGCCGACCCTCTGG
GAGAAAATCCAGCAAGATGCAAGCCTTCAGAATCTGGGATGTTAACCAGAAGACCT
TCTATCTGAGGAACAACCAACTAGTTGCTGGATACTTGCAAGGACCAAATGTCAAT
TTAGAAGAAAAGATAGATGTGGTACCCATTGAGCCTCATGCTCTGTTCTTGGGAATC
CATGGAGGGAAGATGTGCCTGTCCTGTGTCAAGTCTGGTGATGAGACCAGACTCCA
GCTGGAGGCAGTTAACATCACTGACCTGAGCGAGAACAGAAAGCAGGACAAGCGC
TTCGCCTTCATCCGCTCAGACAGTGGCCCCACCACCAGTTTTGAGTCTGCCGCCTGC
CCCGGTTGGTTCCTCTGCACAGCGATGGAAGCTGACCAGCCCGTCAGCCTCACCAAT
ATGCCTGACGAAGGCGTCATGGTCACCAAATTCTACTTCCAGGAGGACGAGTAGTA
ATGTACAAGTAAGCGGCCGCATACATACTTCTTTACATTCCAATACATACTTCTTTA
CATTCCAATACATACTTCTTTACATTCCACCATGGACTAGTACAAACACCATTGTCA
CACTCCAACAAACACCATTGTCACACTCCAACAAACACCATTGTCACACTCCAAAA
GCTTATCGATACCGTCGACTAGAGCTCGCTGATCAGCCTCGACTGTGCCTTCTAGTT
GCCAGCCATCTGTTGTTTGCCCCTCCCCCGTGCCTTCCTTGACCCTGGAAGGTGCCA
CTCCCACTGTCCTTTCCTAATAAAATGAGGAAATTGCATCGCATTGTCTGAGTAGGT
GTCATTCTATTCTGGGGGGTGGGGGGGGCAGGACAGCAAGGGGGAGGATTGGGA
AGACAATTAGGTAGATAAGTAGCATGGCGGGTTAATCATTAACTACAAGGAACCCC
TAGTGATGGAGTTGGCCACTCCCTCTCTGCGCGCTCGCTCGCTCACTGAGGCCGGGC
GACCAAAGGTCGCCCGACGCCCGGGCTTTGCCCGGGCGGCCTCAGTGAGCGAGCGA
GCGCGCAG
>rAAV-scCB6 (hs-amiR33SHN3) EGFP
(SEQ ID NO: 37)
CTGCGCGCTCGCTCGCTCACTGAGGCCGCCCGGGCAAAGCCCGGGCGTCGGGCGAC
CTTTGGTCGCCCGGCCTCAGTGAGCGAGCGAGCGCGCAGAGAGGGAGTGTAGCCAT
GCTCTAGGAAGATCAATTCGGTACAATTCACGCGTCGACATTGATTATTGACTCTGG
TCGTTACATAACTTACGGTAAATGGCCCGCCTGGCTGACCGCCCAACGACCCCGCCC
ATTGACGTCAATAATGACGTATGTTCCCATAGTAACGCCAATAGGGACTTTCCATTG
ACGTCAATGGGTGGAGTATTTACGGTAAACTGCCCACTTGGCAGTACATCAAGTGT
ATCATATGCCAAGTACGCCCCCTATTGACGTCAATGACGGTAAATGGCCCGCCTGG
CATTATGCCCAGTACATGACCTTATGGGACTTTCCTACTTGGCAGTACATCTACTCG
AGGCCACGTTCTGCTTCACTCTCCCCATCTCCCCCCCCTCCCCACCCCCAATTTTGTA
TTTATTTATTTTTTAATTATTTTGTGCAGCGATGGGGGGGGGGGGGGGGGGGGGGGG
GGCGCGCGCCAGGCGGGGCGGGGCGGGGCGAGGGGCGGGGGGGGCGAGGCGGA
GAGGTGCGGCGGCAGCCAATCAGAGCGGCGCGCTCCGAAAGTTTCCTTTTATGGCG
AGGCGGCGGCGGCGGCGGCCCTATAAAAAGCGAAGCGCGCGGCGGGCGGGAGCGG
GATCAGCCACCGCGGTGGCGGCCCTAGAGTCGATCGAGGAACTGAAAAACCAGAA
AGTTAACTGGTAAGTTTAGTCTTTTTGTCTTTTATTTCAGGTCCCAGATCTGGCAGCC
TTGGAGTGGGTTCCTGCCCCCTCGGGCACACAAACAGAGCTGAAGACCACCCTGGG
CACCTCCTTGGCTGGCCGCATACCTCCTGGCGGGCAGCTGTGTACAAACTACTTGAG
AGCAGGTGTTCTGGTGGTACCCACCTGCTCTGTAATAGTTTGTACACAGAGGCCTGC
CTGGCCCTCGAGAGACTGCCCTGACTGAAGGCCCTATCAGGTGGGGGAGGGGATCC
TGATAGAGGGCACTGCTGCCACTGTTGGGGCCCAAGCTGCAGGGGATCCGGTGGTG
GTGCAAATCAAAGAACTGCTCCTCAGTGGATGTTGCCTTTACTTCTAGGCCTGTACG
GAAGTGTTACTTCTGCTCTAAAAGCTGCGGAATTGTACCCGCGGCCGATCCACCGGT
CGCCACCATGGTGAGCAAGGGCGAGGAGCTGTTCACCGGGGTGGTGCCCATCCTGG
TCGAGCTGGACGGCGACGTAAACGGCCACAAGTTCAGCGTGTCCGGCGAGGGCGA
GGGCGATGCCACCTACGGCAAGCTGACCCTGAAGTTCATCTGCACCACCGGCAAGC
TGCCCGTGCCCTGGCCCACCCTCGTGACCACCCTGACCTACGGCGTGCAGTGCTTCA
GCCGCTACCCCGACCACATGAAGCAGCACGACTTCTTCAAGTCCGCCATGCCCGAA
GGCTACGTCCAGGAGCGCACCATCTTCTTCAAGGACGACGGCAACTACAAGACCCG
CGCCGAGGTGAAGTTCGAGGGCGACACCCTGGTGAACCGCATCGAGCTGAAGGGC
ATCGACTTCAAGGAGGACGGCAACATCCTGGGGCACAAGCTGGAGTACAACTACAA
CAGCCACAACGTCTATATCATGGCCGACAAGCAGAAGAACGGCATCAAGGTGAACT
TCAAGATCCGCCACAACATCGAGGACGGCAGCGTGCAGCTCGCCGACCACTACCAG
CAGAACACCCCCATCGGCGACGGCCCCGTGCTGCTGCCCGACAACCACTACCTGAG
CACCCAGTCCGCCCTGAGCAAAGACCCCAACGAGAAGCGCGATCACATGGTCCTGC
TGGAGTTCGTGACCGCCGCCGGGATCACTCTCGGCATGGACGAGCTGTACAAGTAA
AGCGGCCATCAAGCTTATCGATACCGTCGACTAGAGCTCGCTGATCAGCCTCGACT
GTGCCTTCTAGTTGCCAGCCATCTGTTGTTTGCCCCTCCCCCGTGCCTTCCTTGACCC
TGGAAGGTGCCACTCCCACTGTCCTTTCCTAATAAAATGAGGAAATTGCATCGCATT
GTCTGAGTAGGTGTCATTCTATTCTGGGGGGTGGGGTGGGGCAGGACAGCAAGGGG
GAGGATTGGGAAGACAATTAGGTAGATAAGTAGCATGGCGGGTTAATCATTAACTA
CAAGGAACCCCTAGTGATGGAGTTGGCCACTCCCTCTCTGCGCGCTCGCTCGCTCAC
TGAGGCCGGGCGACCAAAGGTCGCCCGACGCCCGGGCTTTGCCCGGGCGGCCTCAG
TGAGCGAGCGAGCGCGCAG
>rAAV-amiR-mCathepsinK
(SEQ ID NO: 38)
ctgcgcgctcgctcgctcactgaggccgcccgggcaaagcccgggcgtcgggcgacctttggtcgcccggcctcagtgagcgagcga
gcgcgcagagagggagtgtagccatgctctaggaagatcaattcggtacaattcacgcgtcgacattgattattgactctggtcgttacataa
cttacggtaaatggcccgcctggctgaccgcccaacgaccccgcccattgacgtcaataatgacgtatgttcccatagtaacgccaatagg
gactttccattgacgtcaatgggtggagtatttacggtaaactgcccacttggcagtacatcaagtgtatcatatgccaagtacgccccctatt
gacgtcaatgacggtaaatggcccgcctggcattatgcccagtacatgaccttatgggactttcctacttggcagtacatctactcgaggcca
cgttctgcttcactctccccatctcccccccctccccacccccaattttgtatttatttattttttaattattttgtgcagcgatgggggcggg
gggggggggggggggggcgcgcgccaggcggggcggggcggggcgaggggggggcggggcgaggcggagaggtgcggcggcag
ccaatcagagcggcgcgctccgaaagtttccttttatggcgaggcggcggcggcggcggccctataaaaagcgaagcgcgcggcggg
cgggagcgggatcagccaccgcggtggcggccctagagtcgatcgaggaactgaaaaaccagaaagttaactggtaagtttagtcttttt
gtcttttatttcaggtcccagatctagggctctgcgtttgctccaggtagtccgctgctcccttgggcctgggcccactgacagccctggtgcc
tctggccggctgcacacctcctggcgggcagctgtgtacaaactacttgagagcaggtgttctggcaatacctgcctgctctgtaatagtttg
tacacggaggcctgccctgactgcccacggtgccgtggccaaagaggatctaagggcaccgctgagggcctacctaaccatcgtgggg
aataaggacagtgtcacccctgcaggggatccggtggtggtgcaaatcaaagaactgctcctcagtggatgttgcctttacttctaggcctgt
acggaagtgttacttctgctctaaaagctgcggaattgtacccgcggccgatccaccggtcgccaccatggGGCAGCCTTGGA
GTGGGTTCCTGCCCCCTCGGGCACACAAACAGAGCTGAAGACCACCCTGGGCACCT
CCTTGGCTGGCCGCATACCTCCTGGCGGGCAGCTGTGtttcatcatagtacacacctcTGTTCTGG
TGGTACCCAgaggtgtgatccatgatgaaaCACAGAGGCCTGCCTGGCCCTCGAGAGACTGCCC
TGACTGAAGGCCCTATCAGGTGGGGGAGGGGATCCTGATAGAGGGCACTGCTGCCA
CTGTTGGGGCCCAAGaagcttatcgataccgtcgactagagctcgctgatcagcctcgactgtgccttctagttgccagccatc
tgttgtttgcccctcccccgtgccttccttgaccctggaaggtgccactcccactgtcctttcctaataaaatgaggaaattgcatcgcattgtct
gagtaggtgtcattctattctggggggtggggggggcaggacagcaagggggaggattgggaagacaattaggtagataagtagcatg
gcgggttaatcattaactacaaggaacccctagtgatggagttggccactccctctctgegcgctcgctcgctcactgaggccgggcgacc
aaaggtcgcccgacgcccgggctttgcccgggcggcctcagtgagcgagcgagcgcgcag
>rAAV-amiR-SHN3+amiR-CathepsinK
(SEQ ID NO: 39)
ctgcgcgctcgctcgctcactgaggccgcccgggcaaagcccgggcgtcgggcgacctttggtcgcccggcctcagtgagcgagcga
gcgcgcagagagggagtgtagccatgctctaggaagatcaattcggtacaattcacgcgtcgacattgattattgactctggtcgttacataa
cttacggtaaatggcccgcctggctgaccgcccaacgaccccgcccattgacgtcaataatgacgtatgttcccatagtaacgccaatagg
gactttccattgacgtcaatgggtggagtatttacggtaaactgcccacttggcagtacatcaagtgtatcatatgccaagtacgccccctatt
gacgtcaatgacggtaaatggcccgcctggcattatgcccagtacatgaccttatgggactttcctacttggcagtacatctactcgaggcca
cgttctgcttcactctccccatctcccccccctccccacccccaattttgtatttatttattttttaattattttgtgcagcgatggggggggg
ggggggggggggggggcgcgcgccaggcggggcggggcggggcgaggggcggggggggcgaggcggagaggtgcggcggcag
ccaatcagagcggcgcgctccgaaagtttccttttatggcgaggcggcggcggcggcggccctataaaaagcgaagcgcgcggcggg
cgggagcgggatcagccaccgcggtggcggccctagagtcgatcgaggaactgaaaaaccagaaagttaactggtaagtttagtcttttt
gtcttttatttcaggtcccagatctagggctctgcgtttgctccaggtagtccgctgctcccttgggcctgggcccactgacagccctggtgcc
tctggccggctgcacacctcctggcgggcagctgtgtttcatcatagtacacacctctgttctggcaatacctggaggtgtgatccatgatga
aacacggaggcctgccctgactgcccacggtgccgtggccaaagaggatctaagggcaccgctgagggcctacctaaccatcgtgggg
aataaggacagtgtcacccctgcaggggatccggtggtggtgcaaatcaaagaactgctcctcagtggatgttgcctttacttctaggcctgt
acggaagtgttacttctgctctaaaagctgcggaattgtacccgcggccgatccaccggtcgccaccatggGGCAGCCTTGGA
GTGGGTTCCTGCCCCCTCGGGCACACAAACAGAGCTGAAGACCACCCTGGGCACCT
CCTTGGCTGGCCGCATACCTCCTGGCGGGCAGCTGTGtacaaactacttgagagcaggTGTTCTG
GTGGTACCCAcctgctctgtaatagtttgtaCACAGAGGCCTGCCTGGCCCTCGAGAGACTGCCC
TGACTGAAGGCCCTATCAGGTGGGGGAGGGGATCCTGATAGAGGGCACTGCTGCCA
CTGTTGGGGCCCAAGaagcttatcgataccgtcgactagagctcgctgatcagcctcgactgtgccttctagttgccagccatc
tgttgtttgcccctcccccgtgccttccttgaccctggaaggtgccactcccactgtcctttcctaataaaatgaggaaattgcatcgcattg
tctgagtaggtgtcattctattctggggggtggggtggggcaggacagcaagggggaggattgggaagacaattaggtagataagtagcatg
gcgggttaatcattaactacaaggaacccctagtgatggagttggccactccctctctgcgcgctcgctcgctcactgaggccgggcgacc
aaaggtcgcccgacgcccgggctttgcccgggcggcctcagtgagcgagcgagcgcgcag
>rAAV-amiR-SHN3+amiR-SOST
(SEQ ID NO: 40)
ctgcgcgctcgctcgctcactgaggccgcccgggcaaagcccgggcgtcgggcgacctttggtcgcccggcctcagtgagcgagcga
gcgcgcagagagggagtgtagccatgctctaggaagatcaattcggtacaattcacgcgtcgacattgattattgactctggtcgttacataa
cttacggtaaatggcccgcctggctgaccgcccaacgaccccgcccattgacgtcaataatgacgtatgttcccatagtaacgccaatagg
gactttccattgacgtcaatgggtggagtatttacggtaaactgcccacttggcagtacatcaagtgtatcatatgccaagtacgccccctatt
gacgtcaatgacggtaaatggcccgcctggcattatgcccagtacatgaccttatgggactttcctacttggcagtacatctactcgaggcca
cgttctgcttcactctccccatctcccccccctccccacccccaattttgtatttatttattttttaattattttgtgcagcgatggggggggg
ggggggggggggggggcgcgcgccaggcggggcggggggggcgaggggggggggggcgaggcggagaggtgcggcggcag
ccaatcagagcggcgcgctccgaaagtttccttttatggcgaggcggcggcggcggcggccctataaaaagcgaagcgcgcggcggg
cgggagcgggatcagccaccgcggtggcggccctagagtcgatcgaggaactgaaaaaccagaaagttaactggtaagtttagtcttttt
gtcttttatttcaggtcccagatctagggctctgcgtttgctccaggtagtccgctgctcccttgggcctgggcccactgacagccctggtgcc
tctggccggctgcacacctcctggcgggcagctgtgtgacctctgtggcatcattcctgttctggcaatacctgggaatgatcgcgcagagg
tcacacggaggcctgccctgactgcccacggtgccgtggccaaagaggatctaagggcaccgctgagggcctacctaaccatcgtggg
gaataaggacagtgtcacccctgcaggggatccggtggtggtgcaaatcaaagaactgctcctcagtggatgttgcctttacttctaggcct
gtacggaagtgttacttctgctctaaaagctgcggaattgtacccgcggccgatccaccggtcgccaccatggGGCAGCCTTGG
AGTGGGTTCCTGCCCCCTCGGGCACACAAACAGAGCTGAAGACCACCCTGGGCACC
TCCTTGGCTGGCCGCATACCTCCTGGCGGGCAGCTGTGtacaaactacttgagagcaggTGTTCT
GGTGGTACCCAcctgctctgtaatagtttgtaCACAGAGGCCTGCCTGGCCCTCGAGAGACTGCC
CTGACTGAAGGCCCTATCAGGTGGGGGAGGGGATCCTGATAGAGGGCACTGCTGCC
ACTGTTGGGGCCCAAGaagcttatcgataccgtcgactagagctcgctgatcagcctcgactgtgccttctagttgccagcca
tctgttgtttgcccctcccccgtgccttccttgaccctggaaggtgccactcccactgtcctttcctaataaaatgaggaaattgcatcgcat
tgtctgagtaggtgtcattctattctggggggtggggggggcaggacagcaagggggaggattgggaagacaattaggtagataagtagcat
ggcgggttaatcattaactacaaggaacccctagtgatggagttggccactccctctctgcgcgctcgctcgctcactgaggccgggcgac
caaaggtcgcccgacgcccgggctttgcccgggcggcctcagtgagcgagcgagcgcgcag

### EQUIVALENTS

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

The terms “about” and “substantially” preceding a numerical value represent ±10% of the recited numerical value.

1. An isolated nucleic acid comprising a transgene comprising an osteoclast (OC)-specific promoter or an osteoblast (OB)-specific promoter operably linked to a nucleic acid sequence encoding one or more inhibitory nucleic acids targeting sclerostin (SOST), schnurri-3 (SHN3), cathepsin K (CTSK), receptor activator of NF-κβ, (RANK) and/or receptor activator of NF-κβ ligand (RANKL).

2. The isolated nucleic acid of claim 1, wherein the transgene further comprises a nucleic acid encoding a protein.

3. (canceled)

4. The isolated nucleic acid of claim 2, wherein the protein comprises soluble human tumor necrosis factor alpha receptor 2 (sTNRF2), a soluble IL-1 Receptor Antagonist (sIL1Rα), or sTNRF2 and sIL1Rα.

5. The isolated nucleic acid of claim 1, wherein the OC-specific promoter comprises a NF-κβ promoter (e.g., RANK promoter) or wherein the OC-specific promoter is induced by inflammation, optionally wherein the NF-κβ promoter is a PB2 promoter (SEQ ID NO: 3).

6. (canceled)

7. The isolated nucleic acid of claim 1, wherein the OB-specific promoter comprises an osteocalcin (OCN) promoter (SEQ ID NO: 4).

8. The isolated nucleic acid of claim 1, wherein at least one of the one or more inhibitory nucleic acids is a shRNA, miRNA, or artificial miRNA (ami-RNA).

9. The isolated nucleic acid of claim 1, wherein at least one of the one or more inhibitory nucleic acids is an ami-RNA comprising a human miRNA backbone, optionally a human miR-33 backbone.

10. The isolated nucleic acid of claim 1, wherein at least one of the one or more inhibitory nucleic acids is an ami-RNA comprising a mouse miRNA backbone, optionally a mouse miR-33 backbone.

11. The isolated nucleic acid of claim 1, wherein at least one of the one or more inhibitory nucleic acids targets SHN3, optionally wherein the inhibitory nucleic acid is encoded by a nucleic acid comprising the sequence set forth in any one of SEQ ID NOs: 8-11.

12. The isolated nucleic acid of claim 1, wherein at least one of the one or more inhibitory nucleic acids targets CTSK, optionally wherein the inhibitory nucleic acid is encoded by a nucleic acid comprising the sequence set forth in any one of SEQ ID NOs: 20-25.

13. The isolated nucleic acid of claim 1, wherein at least one of the one or more inhibitory nucleic acids targets SOST, optionally wherein the inhibitory nucleic acid is encoded by a nucleic acid comprising the sequence set forth in any one of SEQ ID NOs: 26-31.

14. The isolated nucleic acid of claim 1, wherein at least one of the one or more inhibitory nucleic acids targets RANK.

15. The isolated nucleic acid of claim 1, wherein at least one of the one or more inhibitory nucleic acids targets RANKL, optionally wherein the inhibitory nucleic acid is encoded by a nucleic acid comprising the sequence set forth in any one of SEQ ID NOs: 12-19.

16. The isolated nucleic acid of claim 1, wherein the transgene encodes a first inhibitory nucleic acid targeting a gene selected from SHN3, CTSK, SOST, RANK, and RANKL; and a second inhibitory nucleic acid targeting a gene selected from SHN3, CTSK, SOST, RANK, and RANKL.

17. An isolated nucleic acid comprising a transgene encoding a first inhibitory nucleic acid targeting a gene selected from SHN3, CTSK, SOST, RANK, and RANKL; and a second inhibitory nucleic acid targeting a gene selected from SHN3, CTSK, SOST, RANK, and RANKL.

18-29. (canceled)

30. An isolated nucleic acid comprising or encoding a sequence set forth in any one of SEQ ID NOs: 1-40.

31-33. (canceled)

34. A recombinant adeno-associated virus (rAAV) comprising:
(i) the isolated nucleic acid of claim 1; and
(ii) at least one AAV capsid protein.

35.-39. (canceled)

40. A method for inhibiting bone loss in a subject, the method comprising administering to the subject the rAAV of claim 34.

41. A method for inhibiting inflammation in a joint of a subject, the method comprising administering to the subject the rAAV of claim 34.

42. A method for treating rheumatoid arthritis in a subject, the method comprising administering to the subject the rAAV of claim 34.

43-51. (canceled)
